(19) United States
(12) Patent Application Publication
(10) Pub. No.: US 2004/0091964 A1
(43) Pub. Date: May 13, 2004

(54) Modified proteins, isolated novel peptides,and uses thereof

(76) Inventors: Phillip Board, Queanbeyan (AU); Matthew Harris, Murrumbateman (AU)

Correspondence Address: Pillsbury Winthrop, Intellectual Property Group, Suite 200, 11682 El Camino Real, San Diego, CA, 92130-2092, US

(21) Appl. No.: 10/363,112

(22) PCT Filed: Aug. 30, 2001
(86) PCT No.: PCT/AU01/01093

(51) Int. Cl. C07K 14/705; C07H 21/04

(52) U.S. Cl. 435/69.1; 435/320.1; 435/325; 530/350; 536/23.5

(57) ABSTRACT

The present invention provides several modified ABC transporter polypeptides that exhibit novel localization in the plasma membrane of polarized and non-polarized cells. The modified ABC transporter of the invention comprises the amino acid sequence of a native apically targeted ABC transporter, in particular cMOAT, MDR3 or MRP4, wherein the terminal tripeptide T-K-F motif of said native ABC transporter is mutated. The isolated modified ABC transporter polypeptide of the invention, and the nucleotide sequence encoding said polypeptide, have utility in the following applications: First, they are used to induce a drug resistant phenotype in a cell. Second, they are used to protect non-polarized cells during chemotherapy and other therapeutic applications. Third, they are used to produce novel cell lines that are used to screen for novel agonists or antagonists of the corresponding native ABC transporter polypeptides.

### FIELD OF THE INVENTION

The present invention relates generally to novel proteins that are capable of modulating the drug resistance of cells, tissues, organs and whole organisms. More specifically, the present invention provides several modified forms of ATP-Binding Cassette transporter (hereinafter “ABC pump” or “ABC transporter”) polypeptides that are normally localized in the canalicular (apical) membrane of polarized cells where they modulate the transport or efflux of one or more drugs, antibiotics, or other chemical compounds, wherein the modified ABC transporters of the invention are localized in the basolateral membrane of polarized cells, or accumulate in the plasma membrane of a non-polarized cell. Several modified canalicular multispecific organic anion transporter (cMOAT) polypeptides (also known in the art as “MRP2”), a modified MDR3 polypeptide, and a modified MRP4 polypeptide are exemplified herein that are capable of being differentially expressed or localized within the cell membrane compared to the non-modified form of said polypeptides. The modified ABC transporter polypeptides of the invention are further capable of modulating the resistance of cells to a range of compounds, including antibiotics, chemotherapeutic agents, and antifungal compounds, and, accordingly, the present invention clearly extends to the uses of both the isolated modified ABC transporter polypeptide of the invention and the nucleotide sequence encoding same to: (i) induce a multidrug resistant phenotype in a cell; and (ii) protect polarized and non-polarized cells during chemotherapy and other applications. The modified ABC transporter polypeptides of the invention are also particularly useful in screening for compounds that modulate the activity (i.e. agonists or antagonists) of an ABC transporter polypeptide, or to determine if the efflux of a particular compound is modulated by a specific ABC transporter polypeptide. High throughput screening protocols are described herein. The present invention further provides isolated nucleic acids encoding the modified ABC transporter polypeptide and gene constructs comprising same.

General

This specification contains nucleotide and amino acid sequence information prepared using the programme PatentIn Version 3.1, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by descriptor “SEQ ID NO:” followed by the numeric identifier. For example, SEQ ID NO: 1 refers to the information provided in the numeric indicator field designated <400>1, etc.

For the purposes of nomenclature, the nucleotide sequence of the native cMOAT-encoding gene of humans is set forth in SEQ ID NO: 1, and the corresponding amino acid sequence is set forth in SEQ ID NO: 2. The C-terminal portion of native cMOAT is also presented in SEQ ID NO: 37.

The nucleotide sequence of a first modified cMOAT-encoding gene is set forth in SEQ ID NO: 3, and the corresponding amino acid sequence is set forth in SEQ ID NO: 4. The amino acid sequence of SEQ ID NO: 4 corresponds to the ΔcMOAT polypeptide of the invention (also termed herein “ΔT1543 ΔK1544 ΔF1545”), the C-terminal portion of which is presented in SEQ ID NO: 44.

The nucleotide sequence of a second modified cMOAT-encoding gene is set forth in SEQ ID NO: 5, and the corresponding amino acid sequence is set forth in SEQ ID NO: 6. The amino acid sequence of SEQ ID NO: 6 corresponds to the T1543A K1544P F1545V polypeptide of the invention, the C-terminal portion of which is presented in SEQ ID NO: 38.

The nucleotide sequence of a third modified cMOAT-encoding gene is set forth in SEQ ID NO: 7, and the corresponding amino acid sequence is set forth in SEQ ID NO: 8. The amino acid sequence of SEQ ID NO: 8 corresponds to the S1542A polypeptide of the invention, the C-terminal portion of which is presented in SEQ ID NO: 39.

The nucleotide sequence of a fourth modified cMOAT-encoding gene is set forth in SEQ ID NO: 9, and the corresponding amino acid sequence is set forth in SEQ ID NO: 10. The amino acid sequence of SEQ ID NO: 10 corresponds to the T1543A polypeptide of the invention, the C-terminal portion of which is presented in SEQ ID NO: 40.

The nucleotide sequence of a fifth modified cMOAT-encoding gene is set forth in SEQ ID NO: 11, and the corresponding amino acid sequence is set forth in SEQ ID NO: 12. The amino acid sequence of SEQ ID NO: 12 corresponds to the K1544A polypeptide of the invention, the C-terminal portion of which is presented in SEQ ID NO: 41.

The nucleotide sequence of a sixth modified cMOAT-encoding gene is set forth in SEQ ID NO: 13, and the corresponding amino acid sequence is set forth in SEQ ID NO: 14. The amino acid sequence of SEQ ID NO: 14 corresponds to the F1545A polypeptide of the invention, the C-terminal portion of which is presented in SEQ ID NO: 42.

The nucleotide sequence of a seventh modified cMOAT-encoding gene is set forth in SEQ ID NO: 15, and the corresponding amino acid sequence is set forth in SEQ ID NO: 16. The amino acid sequence of SEQ ID NO: 16 corresponds to the T1543A K1544A F1545A polypeptide of the invention, the C-terminal portion of which is presented in SEQ ID NO: 43.

The nucleotide sequence of a modified MDR3-encoding gene is set forth in SEQ ID NO: 48, and the corresponding amino acid sequence of a modified human MDR3 polypeptide of the invention, lacking the T-K-F motif (i.e. the terminal 4 amino acids have been deleted), is presented in SEQ ID NO: 49. The modified MDR3-encoding sequence is amplified from native human MDR3 cDNA using the primer sequences set forth in SEQ ID NO: 59 and SEQ ID NO: 60.

The nucleotide sequence of a modified MRP4-encoding gene is set forth in SEQ ID NO: 50, and the corresponding amino acid sequence of a modified human. MRP4 polypeptide of the invention, lacking the T-K-F motif (i.e. the terminal 3 amino acids have been deleted), is presented in SEQ ID NO: 51. The modified MRP4-encoding sequence is amplified from native human MRP4 cDNA using the primer sequences set forth in SEQ ID NO: 61 and SEQ ID NO: 62.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Reference herein to prior art, including any one or more prior art documents, is not to be taken as an acknowledgment, or suggestion, that said prior art is common general knowledge in Australia or forms a part of the common general knowledge in Australia.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used herein the term “derived from” shall be taken to indicate that a specified integer may be obtained from a particular source alb it not necessarily directly from that source.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

### BACKGROUND TO THE INVENTION

The treatment of bacterial infections, fungal infections, and, more specifically, the treatment of various tumors and/or cancers, often involves the administration of chemotoxins and/or chemostatic compounds, which either kill or inhibit the growth of a tumor, such as, for example, various anti-cancer chemotherapeutic agents, including vinca alkaloids, cisplatin, busulphan (busulfan), vincristine sulphate, merchlorethane, etoposide; and the administration of various chemical compounds which kill a host cell and/or invading pathogens, such as for example, various antibiotic compounds. The majority of cytotoxic drugs are more effective against cells that are rapidly moving through the cell cycle, such as, for example, bacteria that are not in stationary or plateau phases, or tumors having a large growth fraction.

Transport of a drug to a tumor cell (i.e. influx), and its subsequent efflux, are important factors governing the efficacy of any pharmaceutical agent, including a chemotherapeutic agent.

Pharmacokinetic and/or biochemical resistance, including, for example, multidrug resistance (MDR) or pleiotropic resistance, to an administered drug may occur over time. By “resistance”, is meant the ability of a cell, group of cells, tissue, organ, or organism, to remain viable, grow or proliferate in the presence of a chemical compound. Accordingly, a “resistant” cell has the capacity to remain viable, and preferably, to grow and/or to proliferate in the presence of said chemical compound.

Hereinafter, the term “drug resistance” shall be taken to mean pharmacokinetic resistance and/or biochemical resistance, including the phenomenon of MDR, unless specifically stated otherwise.

Drug resistance is generally associated with a low concentration of drug in the target cell, tissue, organ or organism. This is because of decreased intracellular accumulation of the drug, and/or defective transport, and/or reduced absorption, and/or altered drug distribution, and/or biotransformation of the drug, and/or enhanced elimination of the drug from the site of administration and/or effect. The occurrence of drug resistance is one of the major obstacles to the successful treatment of many conditions in humans and animals with such chemotoxins and/or chemostatic compounds, such as, for example, various antibiotics, anti-fungal compounds, anti-viral compounds, and chemotherapeutic agents, in particular, the anthracyclines, epipodophyllotoxins, and vinca alkaloids.

In the case of anti-cancer treatments, drug resistance results in a sensitive tumor being converted into a resistant tumor that no longer responds to chemotherapy.

On the other hand, it is also desirous to protect certain cells, tissues, and organs from cytostatic and/or cytotoxic compounds during treatment of disease, such as, for example, cancer or infection by pathogenic agents. The acquisition of drug resistance in those cells, tissues and organs is a highly desirable outcome. For example, protection of the haematopoietic system during chemotherapy may increase the chances of survival of some cancer patients. Naturally, in such cases it is important to maximize the efflux of the drug from the cells in respect of which protection is sought.

Accordingly, drug resistance can hinder effective treatment.

Various drug resistant cell lines have been identified, such as, for example, rodent cell lines resistant to multiple drugs, and multidrug resistant (MDR) lines derived from the human KB carcinoma cell line (a HeLa subclone). These lines were selected for their resistance to colchicine, vinblastine, or adriamycin (see, for example, Kartner et al. (1983) Science 221,1285-1288; Akiyama et al. (1985) Somatic Cell Mol. Genet. 11, 117-126; Shen et al. (1986) J. Bio. Chem. 261, 7762-7770; Shen et al. (1986) Science, 232: 643-645; and Shen et al., (1986) Mol. Cell. Biol. 6, 4039-4044).

Efflux of a drug through the plasma membrane, which contributes to resistance of the cell, is mediated by one or more specific membrane transporters (Cole and Deeley (1998) Bioessays 20, 931-940; Goftesman et al (1995) Ann. Rev. Genet. 29, 607-649; Higgins et al (1992) Ann. Rev. Cell Biol. 8, 67-113). These membrane transporters belong to the so-called superfamily of ATP-Binding Cassette (ABC) transporters.

Cultured or primary epithelial cells, such as, for example, hepatocytes, neuronal cells, and certain cells of the immune system, maintain a characteristic polarized phenotype. The majority of plasma proteins are distinguishable on the basis of their distribution either to the apical (canalicular) or to the basolateral membrane domain of cultured or primary epithelial cells. Relatively few proteins have been identified that are equally distributed on both membrane domain surfaces of these cells (Mellman et al., (1993) J. Cell Sci., Suppl. 17, 1-7). The ABC transporters are generally targeted to the basolateral membrane of such polarized cells (e.g. MRP1, MRP3, and MRP6). Alternatively, ABC transporters may be targeted to the apical (canalicular) membrane [e.g. the canalicular multispecific organic anion transporter (cMOAT) also known as the multidrug resistance-associated protein 2 (MRP2), the P-glycoprotein (P-gp) transporter and its homologues (e.g. MDR2, MDR3), and MRP4].

Substrates for ABC transporters tend to be amphiphilic organic cations and anions.

Those skilled in the art will be aware that ABC transporters are responsible for the transport of a wide range of compounds, such as, for example, 4-NQO, sorbic acid, ketoconazole, econazole, oligomycin, antimycin, paromomycin, colchicine, vinblastine, and adriamycin.

It has also been shown that many pathogenic microorganisms, such as Candida albicans and Plasmodium falciparum, can use the ABC transporter-mediated drug efflux mechanism to evade the toxicity of an administered therapeutic agent (Cowrnan and Foote (1990) Int. J. Parasitol. 20, 503-513; Foote et al (1989) Cell 57, 921-930; Prasad et al. (1995) Curr. Genet 27, 320-329; Sanglard et al (1995) Antimicrob. Agents Chemother. 39, 2378-2386), or to otherwise develop resistance.

The P-gp, MRP1, MRP2 (cMOAT), MRP3, MRP4, MRP5, MRP6, MDR2, and MDR3 proteins are all membrane-localized proteins that pump drugs out of cells by an energy-dependent mechanism requiring ATP. In the liver, P-gp, MRP2 (cMOAT), MRP4, and MDR3, at least, transport a range of organic compounds across the apical (canalicular) membrane into bile. In non-polarized cells, MRP2 (cMOAT) accumulates in intracellular vesicles, with little accumulation of this protein in the plasma membrane (Harris et al., (2001) J. Biol. Chem 24, 20876-20881). In contrast to these so-called “apical” ABC transporter proteins, the MRP1, MRP3, and MRP6 proteins normally function in the basolateral (sinusoidal) membrane of polarized cells. Increased activity of the ABC transporters may lower the intracellular accumulation of a particular drug in all cells in which they are expressed, and result in the cell becoming resistant to the administered drug.

Several human P-glycoproteins (P-gp), such as, for example, MDR1, MDR2, and MDR3, have been identified. The genes encoding these proteins are homologous to the hamster mdr gene (see, for example, Roninson et al. (1984) Nature, 309, 626-628; Gros et al., (1986) Nature 323, 728-731 and Gros et al. (1986) Proc. Natl. Acad Sci. USA, 83, 337-341). The MDR1 and MDR2 proteins are expressed in multidrug-resistant human KB carcinoma cell lines (Fojo et al., (1985) Proc. Natl. Acad. Sci. USA 82, 7661-7665; Roninson et al (1986) Proc. Natl. Sci. USA 83, 4538-4542). The MDR1 gene encodes a 4.5-kb mRNA which is over expressed in all of the highly drug-resistant cell lines (Roninson et al. (1986) Proc. Natl. Sci. USA 83, 4538-4542; Shen et al. (1986) J. Bio. Chem. 261, 7762-7770; Shen et al. (1986) Science, 232: 643-645; and Shen et al., (1986) Mol. Cell. Biol. 6, 4039-4044), and in certain normal and tumor tissues (Fojo et al., (1987) Proc. Natl. Acad. Sci. USA 84, 265-269). Moreover, Chen et al., (1987) Cell 47, 381-389 have described the isolation of a set of overlapping cDNAs for the entire coding region of the human MDR1 mRNA. The human MDR1 gene is also expressed at high levels in murine cells that have been transformed to a drug resistant phenotype using genomic DNA derived from drug resistant human cells. This finding suggests that expression of the MDR1 gene can contribute to the development of the MDR phenotype (Shen et al (1986) J. Bio. Chem. 261, 7762-7770; Shen et al. (1986) Science, 232: 643-645; and Shen et al., (1986) Mol. Cell. Biol. 6, 4039-4044). Several additional workers have demonstrated the ability of an isolated, and expressed, murine mdr gene to confer multidrug resistance on isolated cells (Gros et al. (1986) Proc. Natl. Acad. Sci. USA, 83, 337-341; Gros et al., (1986) Nature 323, 728-731; Ueda et al. (1987) J. Biol. Chem. 262, 505-508).

Native P-glycoprotein is absent from most normal tissues, but a variety of tissues in mammals have been found to express P-gp in an inducible form, such as, for example, the kidney, liver, small intestine, colon, uterine secretory epithelium, and adrenal gland. In polarized cells, such as those in the renal tubule or small intestinal mucosa, liver, and pancreas, P-gp is expressed in a polarized manner and is located in the luminal brush borders. Thus, P-gp is located on the apical surface of proximal tubule cells in the kidney, on the apical surface of intestinal epithelial cells, on the apical surface of small ductules of the pancreas and on the binary face of hepatocytes. Only in adrenal cells is P-gp is uniformly distributed in the membrane. The normal function of P-gp is not firmly established, but it is known that it can remove toxic substances from cells (Gatmaitan and Arias (1993) Adv Pharmacol 24:77-97).

P-gp is phosphorylated in vivo, and early studies have demonstrated that a change in the state of phosphorylation of P-gp has been associated with differences in relative drug resistance of mammalian cells, suggesting that the phosphorylation mechanisms may be involved in the regulation of the efflux activity of the drug transporter (Center (1983) Biochem. Biophys. Res. Comm. 115, 159-166; Hamada et al (1987) Cancer Res. 47, 2860-2865).

P-gp-mediated drug resistance may be ameliorated to some extent via the administration of P-gp modulators or antagonists that inhibit the export function of P-gp, thereby allowing the accumulation of a chemotherapeutic agent administered to the patient. However, P-gp modulators are not useful in combination therapy for the simultaneous protection of the haematopoietic system and anti-cancer treatment of the patient, particularly where MDR1 is ectopically expressed in haematopoietic cells and chemotherapeutic agents and P-gp modulators are administered to inhibit or prevent tumorigenesis. This is because the P-gp modulator inhibits the activity of ectopically expressed MDR1 protein, in addition to inhibiting the endogenous P-gp activity.

The cMOAT transporter activity was initially characterized in hepatocytes, by comparing normal rats to mutants (TR/GY) that lacked canalicular transport activity (Oude Elferink, et aL (1995) Biochim Biophys Acta. 1241, 215-268). Evers, R., et al. (J Clin Invest. (1988) 101, 1310-1319) demonstrated that the drug export activity of recombinant cMOAT protein in polarized kidney MDCK cells expressing a cMOAT-encoding cDNA was confined predominantly to the apical membrane. Subsequently, immunostaining also revealed that the cMOAT protein is predominantly expressed in the apical membrane of hepatocytes (Konig et al. (1999) Biochim Biophys Acta. 1461, 377-394).

The present inventors have also discovered that native cMOAT fails to accumulate in the plasma membrane of non-polarized cells (Harris et al., (2001) J. Biol. Chem 24, 20876-20881). Based upon this expression pattern, the native cMOAT polypeptide is of limited utility in conferring drug resistance on non-polarized cells, such as, for example, certain cells of the haematopoietic system.

Native MRP1 transporter activity is enhanced in tumors exposed to chemotherapeutic agents, thereby conferring acquired resistance on the tumor cells (Goldstein, et al. (1989) J Natl Cancer Inst. 81,116-124; Slapak, C. A., et al. (1994) Blood 84, 3113-3121).

Phosphorylation has been proposed to regulate activity of the S. cerevislae Ycf1 protein. Ycf1 is an orthologue of human MRP1 located on the vacuolar membrane of yeast cells (Li et al (1998) J. Biol. Chem. 273, 33449-33454; Szczpka et al (1994) J. Biol. Chem. 269, 22853-22857).

For the treatment of some cancerous cells, such as those of non-small cell lung cancers, MRP1-mediated drug resistance in respect of chemotherapeutic agents is not acquired. Rather, resistance occurs from the outset of treatment (i.e. intrinsic resistance), indicating a high constitutive level of expression of the MRP1 protein (Zaman; G. J., et al., (1993) Cancer Res. 53, 1747-1750). As a consequence, the treatment of patients having advanced tumors, relapsed tumors, or tumors which exhibit intrinsic MRP1-mediated resistance, often requires high doses of chemotherapeutic agent(s). The potential benefits of such high-dosage regimens are generally offset or compromised by myelosuppression, involving the destruction of bone marrow cells, that is induced by the cytotoxic chemotherapeutic agent used.

Protection of bone marrow from the cytotoxic effects of chemotherapeutic agents has been attempted in murine models. For example, MDR1 has been expressed ectopically in murine bone marrow cells. However, in such procedures, myeloproliferative syndrome develops in the mice, wherein cells of certain haematopoietic lineages differentiate and proliferate abnormally (Bunting, K. D., et al., (1998) Blood 92, 2269-2279).

### SUMMARY OF THE INVENTION

In work leading up to the present invention, the inventors sought to identify novel means for modulating the drug resistance of cells mediated by ABC transporter polypeptides, so as to provide for improved treatment regimes and/or to reduce the adverse side-effects of drugs on the haematopoietic system. The inventors have produced a modified ABC transporter polypeptide having novel distribution characteristics in the plasma membrane of polarized and non-polarized cells. These novel distribution characteristics facilitate the treatment of cells by gene therapy regimes, including the use of combination therapies involving both gene technology and traditional drug administration regimes.

More particularly, the present invention provides modified ABC transporter polypeptides, including modified cMOAT, MDR3 and MRP4 polypeptides, that are capable of being predominantly translocated to the basolateral (sinusoidal) membrane, or localized in the plasma membrane of a non-polarized cell. The modified polypeptide thus exhibits a surprising and novel accumulation relative to the corresponding native ABC transporter polypeptide.

Preferably, the modified ABC transporter polypeptide of the present invention consists of an active ABC transporter polypeptide comprising a mutation wherein at least one amino acid residue in the C-terminal region of said active ABC transporter polypeptide is substituted or deleted.

In an alternative embodiment, the modified ABC transporter polypeptide of the present invention consists of an active ABC transporter polypeptide comprising a mutation wherein at least one amino acid residue of a tripeptide T-K-F motif present in said active ABC transporter polypeptide is substituted or deleted.

The novel localization patterns for the modified ABC transporter polypeptides described herein facilitates the efflux of certain ligand drugs from the cell to confer resistance properties thereon. The present invention clearly extends to any and all uses of the novel modified ABC transporter polypeptides as described herein consistent with their stated modes of action.

In particular, the modified ABC transporter polypeptide confers resistance to one or more chemical compounds on a cell. For example, resistance is conferred to a cytostatic or cytotoxic compound used in the treatment of infection or disease. More particularly, the modified polypeptides are useful when protection of non-polarized cells (e.g. cells of the haematopoietic system) is required during the treatment of patients with cytotoxic or cytostatic compounds.

In fact, the modified ABC transporter of the invention is useful for conferring resistance against any pharmaceutical agent that that is metabolized by ABC transporters that are normally apically-localized in polarized cells, by facilitating efflux through the basolateral membrane.

Alternatively, or in addition, the modified ABC transporter of the invention is useful for conferring de novo resistance on a non-polarized cell by facilitating efflux through the plasma membrane. In a particularly preferred embodiment exemplified herein, resistance to Busulfan is conferred on L1210 cells by ectopically expressing a modified cMOAT polypeptide therein.

As a consequence of this conferred resistance, the modified ABC transporter can be used to identify any potentially toxic agents at an early stage, by screening chemical libraries, thereby identifying novel cytotoxins that would not otherwise be identified prior to clinical trials or use. Once identified, the correct dosage level of any pharmaceutical compound for a particular cell type or genetic background, to achieve a desired effect (e.g. toxicity) is readily determined.

Additionally, the modified ABC transporter polypeptide of the invention is used in combination with modulators of heterologous ABC transporters.

Additionally, the modified ABC transporter polypeptide of the invention is used to develop novel cell lines for assaying ABC transporter activity, substrate specificity, drug metabolism, or drug transport.

The assays supra are particularly amenable to identifying new pharmaceuticals that modulate ABC transporter activity. Accordingly, a further aspect of the invention contemplates a simple and reliable in vivo screening system for the discovery of novel agonists and antagonists of an ABC transporter polypeptide. Additionally the screening system can be used to determine if efflux by a certain ABC transporter is a significant pathway in the metabolism of a particular drug.

A further aspect of the present invention provides a gene construct comprising a nucleotide sequence encoding the modified ABC transporter polypeptide of the invention. Preferably, the nucleic acid molecule is operably linked to a promoter sequence to facilitate its expression in a bacterial cell, yeast, fungal cell, insect cell, or mammalian cell.

The gene construct according to this embodiment of the invention is particularly useful for conferring novel drug resistance characteristics on a cell, in particular a non-polarized cell, or alternatively, for transporting particular drugs from the cell. Accordingly, a further aspect of the invention provides a cell comprising the subject gene construct and preferably, which expresses the modified ABC transporter polypeptide of the invention.

In a particularly preferred embodiment, non-polarized cells, (e.g. fibroblasts or cells of the haemopoietic system) are produced that express the modified ABC transporter polypeptide generally within the plasma membrane where it functions in the efflux of certain ligand drugs from the cell to confer resistance properties thereon.

Alternatively, polarized cells, (e.g. cultured epithelial cells such as MDCK or Caco-2 cells, or primary epithelial cells such as hepatocytes, intestinal cells, or hippocampal neurons) are produced that express the modified ABC transporter polypeptide predominantly in the basolateral membrane.

A further aspect of the invention contemplates a transport signal peptide to facilitate the efficient translocation or transcytosis of a polypeptide to the apical membrane of a polarized cell.

### BRIEF DESCRIPTION OF THE DRAWINGS

FIG. 1 is a copy of a photographic representation of a representative Madine-Darby canine kidney (MDCK) cell expressing cMOAT-gfp in a confluent monolayer of cells. Fluorescence is evident throughout the cell in the top down view (upper panel). However, in cross-section, the XZ view reveals specific apical (AP) localization and minimal basolateral (BL) targeting of protein (lower panel). The cover-slip is detected as a line on the apical surface of the cells due to autofluorescence. All scale bars indicate 5 microns.

FIG. 2 is a copy of a photographic representation showing that confluent MDCK cells expressing MRP1-gfp have a ringed appearance in the top down view (upper panel) due to fluorescence in the basolateral (BL) membrane. In the XZ view (lower panel) the lateral targeting of MRP1-gfp is confirmed with the cell to cell membranes being defined. AP, apical.

FIG. 3 is a copy of a photographic representation showing that confluent MDCK cells expressing ΔcMOAT appear ringed in the top down view (upper panel) with a similar appearance to MRP1-gfp (FIG. 2). In the XZ view (lower panel), ΔcMOAT-gfp shows definite lateral localization with the cell to cell membrane outlined by fluorescing protein. Apical (AP) targeting is minimal compared with native cMOAT fused to GFP (FIG. 1). BL, basolateral.

FIG. 4 is a copy of a photographic representation showing the localization of modified cMOAT-gfp fusion proteins comprising mutations of the T-K-F motif of the cMOAT portion. Upper panels in each figure represent the top down view of the cells, whilst the lower panels represent the XZ view of cells, as follows.:

FIG. 4A is a copy of a photographic representation showing that the T1543A mutant has a non-polarized distribution of the fusion protein. Fluorescence was detected in both the apical (AP) and basolateral (BL) membranes giving a ringed appearance from the top down view (upper panel), but the XZ view (lower panel) reveals the non polarized distribution. The intracellular fluorescence is due to background autofluorescence and not GFP.

FIG. 4B is a copy of a photographic representation showing that the K1544A mutant also lost polarized distribution of the fusion protein, with the protein being detected in the apical and basolateral membranes.

FIG. 4C is a copy of a photographic representation showing that the F1545A mutant has the same localization as the native protein.

FIG. 4D is a copy of a photographic representation showing that the triple mutant (i.e. T1543A K1544A F1545A) is localized apically in the top down view (upper panel) however distributed in both the apical and basolateral membranes in the XZ view (lower panel), indicating a non-polarized distribution.

FIG. 4E is a copy of a photographic representation showing that the S1542A mutant exhibits a less distinct distribution wherein the plasma membrane was outlined by the fluorescence of the protein, but on closer inspection in the XZ view (lower panel), the fluorescence appears to be in sub-membrane vesicles.

FIG. 5 is a copy of a photographic representation showing the distribution of cMOAT polypeptides in L1210 cells, as follows:

FIG. 5A is a copy of a photographic representation showing L1210 cells that were transiently transfected with cMOAT-gfp. The majority of cMOAT-gfp accumulated in intracellular vesicles with minimal plasma membrane localization.

FIG. 5B is a copy of a photographic representation showing L1210 cells that were transiently transfected with ΔcMOAT-gfp. The majority of ΔcMOAT-gfp localized to the cell membrane.

FIG. 5C is a copy of a photographic representation showing M2 III6 antibody binding to cMOAT in L1210 cells. Native cMOAT was detected in intracellular vesicles surrounding the nucleus (N). This localization is consistent with cMOAT-gfp localization.

FIG. 5D is a copy of a photographic representation showing M2 III6 antibody binding to ΔcMOAT in L1210 cells. ΔcMOAT was detected in the cell membrane confirming the effects of the TKF motif deletion found with ΔcMOAT-gfp.

FIG. 6 is a graphical representation showing the efflux of DNP-GS into the supernatant by L1210 cells at specific time intervals, as determined by spectrophotometry (Olive et al (1994) Biochim. Biophys. Acta. 1224, 264-268). The control L1210 cells (o) and those cells that were transfected with wild type cMOAT (▪) had the same rate of efflux. The L1210 cells expressing ΔcMOAT (▴) had increased transport of the DNP-GS into the extracellular medium. The background transport of DNP-GS is due to constitutive MRP1. Results are the mean of three separate experiments ± the S.D.

FIG. 7 is a graphical representation of an amino acid sequence alignment of the C terminal regions of ABC transporter proteins from a number of species with the HisP protein. This alignment is derived from an alignment of the entire C-terminal cytoplasmic domain of 37 ABC transporters. The cMOAT homologues have a distinct C-terminal extension when compared with the basolaterally targeted proteins MRP1, MRP3, and MRP6. The sequences presented in the alignment are the C-terminal portions of the following naturally-occurring ABC transporter proteins: HisP (SEQ ID NO: 45); human cMOAT (SEQ ID NO: 17); mouse cMOAT (SEQ ID NO: 46); rat cMOAT (SEQ ID NO: 18); rabbit cMOAT (SEQ ID NO: 19); human P-gp (SEQ ID NO: 20); rat P-gp (SEQ ID NO: 21); human MDR3 (SEQ ID NO: 22); human MRP1 (SEQ ID NO: 23); and human MRP4 (SEQ ID NO: 47). The TKF motif of each sequence is in bold type.

FIG. 8 is a copy of a photographic representation of a homology model of the C-terminal domains of native MRP1 (top panel) and native cMOAT (lower panel), based on the crystal structure of HisP. The view is looking down on the subunit from the membrane into the cytoplasm. The lower face is the C-terminal helix (marked “C-terminus” in each panel). The C-terminal helix of native cMOAT is clearly longer than in native MRP1. The T-K-F motif sits at the end of the C-terminal helix of cMOAT.

FIG. 9 is a graphical representation showing the enhanced resistance of L1210 cells expressing ΔcMOAT (i.e. SEQ ID NO: 4) to the chemotherapeutic agent Busulfan. Cells were incubated with a range of concentrations of Busulfan (x-axis) and the percentages of cells surviving were determined, as indicated by the ordinate. Cells were either wild type cells (♦); L1210 cells expressing native cMOAT (-▪-); or L1210 cells expressing ΔcMOAT (-Δ-). The best-ft exponential curve for L1210 cells expressing ΔcMOAT is also indicated. L1210 cells expressing ΔcMOAT had at least a 2-fold higher IC₅₀ for Busulfan than the other cells tested.

### DETAILED DESCRIPTION OF THE PREFERRED EMBODIMENTS

One aspect of the present invention provides a modified ABC transporter polypeptide having a novel distribution in the plasma membrane of a cell compared to the corresponding native ABC transporter polypeptide. It will be apparent from the preceding description that ABC transporter polypeptides may have differential localization within the apical membranes of polarized and non-polarized cells. For example, native cMOAT, native MRP4, native P-gp, and native P-gp homologues (MDR2 and MDR3) are generally found in the apical membrane domain of a polarized cell, such as hepatic cells. The native transporters thus function to transport organic anions across the canalicular membrane into bile. The native polypeptides are also localized intracellularly in non-polarized cells. In contrast to these apical proteins, the MRP1, MRP3, and MRP6 polypeptides of humans are localized to the basolateral membrane domain of polarized cells.

Accordingly, in a preferred embodiment, the present invention encompasses modified forms of those ABC transporter polypeptides that are normally found in the apical membrane of polarized cells. In a particularly preferred embodiment, the modified ABC transporter polypeptide of the invention is a modified cMOAT polypeptide, modified MDR3 polypeptide, or modified MRP4 polypeptide.

Preferably, the native ABC transporter polypeptide from which the modified ABC transporter polypeptide is derived is a polypeptide of a human or non-human mammal, such as, for example, a human, rat, rabbit, or mouse. In a particularly preferred embodiment, the polypeptide is from humans.

In accordance with this embodiment, it is particularly preferred that the modified ABC transporter polypeptide of the invention consist of an amino acid sequence presented in any one of SEQ ID NOs: 4, 6, 10, 12, 16, 48, or 49. A full description of each of said amino acid sequences is presented inter alia at pages 24 of the specification. Means for the production of these modified ABC transporter polypeptides will be apparent from the exemplified subject matter described herein.

The modified ABC transporter polypeptide of the invention is capable of accumulating in the plasma membrane of a polarized cell, however in contrast to the naturally-occurring form, the modified ABC transporter polypeptide of the present invention is capable of being distributed predominantly to the basolateral membrane of a polarized cell.

By “predominantly to the basolateral membrane” is meant that most of said modified ABC transporter polypeptide is found in the basolateral membrane of polarized cells. Preferably, more than about 70% of the modified ABC transporter is found in the basolateral membrane, and more preferably, more than about 80%, and even more preferably, about 90% of the modified ABC transporter polypeptide is localized in the basolateral membrane of polarized cells.

Polarized cell types will be well known to those skilled in the art. These include, for example, cultured epithelial cells such as MDCK cells, Caco-2 cells, and primary epithelial cells such as those cells of hepatic and intestinal lineage, such as, for example, cells of the kidney, including the renal tubule; the liver; small intestine, including the small intestinal mucosa; liver; and pancreas.

In an alternative embodiment, the modified ABC transporter polypeptide of the present invention accumulates in the plasma membrane of a non-polarized cell. The key observation by the inventors that the modified ABC transporter polypeptide of the invention accumulate in the plasma membrane of non-polarized cells is surprising and unexpected in view of the absence of detectable accumulation of the naturally-occurring form in the plasma membranes of such cells.

Non-polarized cell types will be well known to those skilled in the art. These include, for example, non-epithelial cells such as those forming the haematopoietic system and cultured cell types such as L1210 cells and Jurkat cells.

Preferably, the modified ABC transporter polypeptide of the present invention consists of an active ABC transporter polypeptide comprising a mutation wherein at least one amino acid residue in the C-terminal region of said active ABC transporter polypeptide is substituted or deleted.

In the present context, the term “C-terminal region” or a similar term, such as, for example, “C-terminus”, shall be taken to mean a portion comprising at least the C-terminal 20 amino acids of the corresponding native or naturally-occurring ABC transporter polypeptide. Preferably a “C-terminal region” comprises at least the C-terminal 10 amino acids of an ABC transporter polypeptide, and even more preferably at least the C-terminal 5 amino acids of an ABC transporter polypeptide.

In a particularly preferred embodiment, a sequence comprising three amino acid residues in the C-terminal region of a naturally occurring ABC transporter polypeptide is mutated or deleted. As will be apparent from the subject matter described herein, a “C-terminal region” generally includes an amino acid sequence comprising a T-K-F-motif.

The term “T-K-F motif”, or similar term, shall be taken to refer to an amino acid sequence derived from the amino acid sequence of an ABC transporter polypeptide normally present in the apical membrane of a polarized cell, wherein said amino acid sequence is selected from the group consisting of:

(i) threonine-lysine-phenylalanine (i.e. T-K-F) (SEQ ID NO: 52);

(ii) threonine-alanine-phenylalanine (i.e. T-A-F)(SEQ ID NO: 53);

(iii) threonine-alanine-lysine (i.e. T-A-L) (SEQ ID NO: 54);

(iv) threonine-glutamate-leucine (i.e. T-E-L) (SEQ ID NO: 55);

(v) threonine-lysine-arginine (i.e. T-K-R) (SEQ ID NO: 56);

(vi) threonine-glutamine-asparagine (i.e. T-Q-N) (SEQ ID NO: 57); and

(vii) alanine-lysine-arginine (i.e. A-K-R) (SEQ ID NO: 58).

In this respect, the present inventors have demonstrated that a T-K-F motif as defined herein above is present in a number of ABC transporter polypeptides that normally accumulate predominantly in the apical membrane of a polarized cell. It will also be understood that a T-K-F motif is not present in the C-terminal region of an ABC transporter that normally accumulates predominantly in the basolateral membrane of a polarized cell.

Surprisingly, mutation or deletion of the T-K-F motif of cMOAT, MDR3, or MRP4 alters the spatial accumulation of the modified ABC transporter polypeptide within the plasma membrane of both polarized and non-polarized cells. More particularly, mutation or deletion of the T-K-F motif produces a modified ABC transporter polypeptide capable of accumulating in the plasma membrane of a non-polarized cell or predominantly in the basolateral membrane of a polarized cell. These modified patterns of accumulation have utility in the field modifying the drug resistance of polarized and non-polarized cell types.

In an alternative embodiment, the modified ABC transporter polypeptide of the present invention consists of an active ABC transporter polypeptide comprising a mutation wherein at least one amino acid residue of a tripeptide T-K-F motif present in said active ABC transporter polypeptide is substituted or deleted. Preferably at least two amino acid residues of the T-K-F motif is substituted or deleted. More preferably, all three amino acid residues of the T-K-F motif are deleted or substituted. As will be apparent from the preceding description, such a substitution or deletion modifies the localization of the modified ABC transporter polypeptide within the plasma membrane of both polarized and non-polarized cells.

The modified ABC transporter polypeptide may be a synthetic peptide produced by any method known to those skilled in the art, such as by using Fmoc chemistry. Alternatively, a modified ABC transporter polypeptide may be produced by recombinant means, wherein nucleic acid encoding a native ABC transporter polypeptide is subjected to mutagenesis and the mutated sequence is expressed in a cell to produce the modified ABC transporter polypeptide.

Substitutions encompass any amino acid alterations in which an amino acid is replaced with a different conventional or non-conventional amino acid residue. To produce the modified ABC transporter polypeptide of the invention, amino acids in the C-terminal region of a native ABC transporter polypeptide may be substituted for other conventional or non-conventional amino acids having different properties. For example the new amino acid may have a different property to the base amino acid that is selected from the group consisting of hydrophobicity, hydrophilicity, hydrophobic moment, antigenicity, and propensity to form or break α-helical structures or β-sheet structures.

Conventional amino acid residues contemplated herein are described in Table 1. Non-conventional amino acid residues contemplated herein are described in Table 2.

Substitutions encompassed by the present invention will generally be “non-conservative”. This means that an amino acid residue which is present in a native ABC transporter polypeptide is substituted with an amino acid having a different property. Such non-conservative substitutions generally involve a substitution for an amino acid from a different group to the base amino acid. For example a non-charged residue can be substituted for a charged residue, or a hydrophobic residue can be substituted for alanine.

Particularly preferred amino add substitutions are selected from the group consisting of Ser [symbol] Ala; Thr [symbol] Ala; Lys [symbol] Ala or Pro; and Phe [symbol] Ala or Val.

Amino acid substitutions may be of multiple residues, either clustered or dispersed, within the C-terminal region, and preferably are positioned within the T-K-F motif of the native ABC transporter polypeptide or immediately adjacent thereto. Accordingly, the clustered substitution of Thr-Lys-Phe (i.e. the T-K-F motif) for Ala-Ala-Ala is clearly within the scope of this invention.

Amino acid deletions are those mutations wherein one or more amino acid residues within the C-terminal region of an ABC transporter polypeptide including the T-K-F motif, are removed. Amino acid deletions will usually be of the order of about 1-10 amino acid residues.

Amino acid insertions are those mutations wherein one or more amino acid residues are added to C-terminal region of an ABC transporter polypeptide, preferably disrupting the T-K-F motif.

TABLE 1

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |

In a particularly preferred embodiment of the invention, 14 amino acid residues is deleted from the C-terminus of a cMOAT polypeptide, P-gp polypeptide, MDR3 polypeptide, or MRP4 polypeptide, to produce a modified ABC transporter polypeptide. Alternatively, at least the first of second amino acid residue of the presumptive T-K-F motif is deleted or substituted. As exemplified herein for cMOAT, mutation or deletion of T1543 and/or K1544, optionally further including a mutation or deletion of F1545, significantly modifies protein targeting. Also exemplified herein, deletion of the entire T-K-F motif of cMOAT, MDR3, or MRP4 modified cellular localization of the protein.

Accordingly, a particularly preferred embodiment of the invention provides a modified ABC transporter polypeptide consisting of a modified cMOAT polypeptide having an amino acid sequence substantially as set forth in any one of SEQ ID NOs: 4, 6, 10, 12, 16, 48, or 49, or a functional variant thereof having up to 5 amino adds removed from the C-terminal region and preferably, having as many as 10-20 amino acids removed from the C-terminal region of the corresponding native protein. In the present context, the term “functional variant” means any modified ABC transporter polypeptide that has the transport function of a native ABC transporter polypeptide notwithstanding that it is localized in a different membrane domain to the native ABC transporter polypeptide.

This aspect of the invention clearly includes any fusion protein comprising the modified ABC transporter, particularly a fusion polypeptide between the modified ABC transporter and green fluorescent protein (GFP) as exemplified herein.

A second aspect of the invention clearly extends to the isolated nucleic acid encoding the modified ABC transporter polypeptide described herein.

This aspect of the invention relates to a nucleic acid molecule consisting of a nucleotide sequence encoding a functional ABC transporter polypeptide, wherein a native ABC transporter polypeptide-encoding nucleotide sequence has a mutation selected from the group consisting of:

(i) a deletion of at least nine nucleotides from the 3′-end of the coding region of the wild-type gene sequence;

(ii) a deletion from the 3′-end of the coding region of the wild-type gene sequence which removes at least a part of the nucleotide sequence of said gene encoding the T-K-F motif;

(iii) a substitution within the 3′-end of the coding region of the wild-type gene which mutates the nucleotide sequence of said gene encoding the T-K-F motif;

(iv) an insertion within the 3′-end of the coding region of the wild-type gene which mutates the nucleotide sequence of said gene encoding the T-K-F motif; and

(v) any mutation that introduces a stop codon within the 3-end of the coding region of the wild-type gene thereby preventing nucleotide sequences encoding TKF motif of said gene from being translated.

Preferably, the deletion referred to in sub-paragraph (i) supra comprises a deletion of at least about 10 nucleotides, more preferably, at least about 11 nucleotides, and more preferably at least about 12 nucleotides from the 3′-end of the coding region of the corresponding native ABC transporter polypeptide-encoding nucleotide sequence.

In a particularly preferred embodiment, the isolated nucleic acid of the invention consists of the nucleotide sequence of the modified cMOAT-encoding gene set forth in any one of SEQ ID NOs: 3, 5, 9, 11, or 15.

In an alternative embodiment, nucleic acid encoding a modified ABC transporter polypeptide is produced by amplification using primers containing mutations therein, as described in the examples. As will be known to those skilled in the art, the amplified mutant sequence will include the nucleotide sequence of the primer, or the complementary sequence thereto at the 3′-end of its coding region. Accordingly, the present invention clearly encompasses a modified ABC transporter that includes a nucleotide sequence selected from the group consisting of SEQ ID Nos: 26 to 33, 37, 59-62, and a complementary nucleotide sequence to any one of said SEQ ID NOs.

To express the modified ABC transporter polypeptide of the present invention in a cell, such as a mammalian cell, it is desirable to place the nucleic acid molecule in an expressible format in operable connection with a suitable promoter sequence.

As used herein, a “nucleic acid molecule in an expressible format” comprises the protein-encoding region in operable connection with a promoter or other regulatory sequence capable of regulating expression of the modified ABC transporter polypeptide encoded by said protein-encoding region. As will be known tot hose skilled in the art, such expression is generally carried out in an appropriate cell host.

Reference herein to a “promoter” is to be taken in its broadest context to include the transcriptional regulatory sequences of a classical genomic gene. Such regulatory sequences include the TATA box which is required for accurate transcription initiation, with or without a CCAAT box sequence and additional regulatory elements (i.e., upstream activating sequences, enhancers and silencers) that alter gene expression in response to developmental and/or external stimuli, or in a tissue-specific manner. In the present context, the term “promoter” is also used to describe a recombinant, synthetic or fusion molecule, or derivative that is capable of conferring, activating or enhancing expression of nucleic acid encoding the modified ABC transporter polypeptide of the invention. Preferred promoters can contain additional copies of one or more specific regulatory elements to further enhance expression and/or to alter the spatial expression and/or temporal expression of the said nucleic acid molecule.

Placing a nucleic acid molecule under the regulatory control of (i.e., “in operable connection with”) a promoter sequence means positioning the said molecule such that expression is controlled by the promoter sequence. Promoters are generally, but not necessarily, positioned 5′ (upstream) to the genes that they control. To produce a heterologous promoter/structural gene combination, the promoter is generally positioned at a distance from the gene transcription start site that is approximately the same as the distance between that promoter and the gene it controls in its natural setting. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Preferably, the promoter sequence facilitates expression of the modified ABC transporter polypeptide in a bacterial cell, yeast, fungal cell, insect cell, or mammalian cell.

The prerequisite for producing intact polypeptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (1987). In: Current Protocols in Molecular Biology. Wiley Interscience (ISBN 047150338) or Sambrook et al (1989) Molecular Cloning: A Laboratory Manual, Cold Spring Harbor Laboratory, Cold Spring Harbor, N.Y. Numerous plasmids with suitable promoter sequences for expression in bacteria and efficient ribosome binding sites have been described, such as for example, pKC30 (λ_{L}: Shimatake and Rosenberg (1981) Nature 292, 128), pKK173-3 (tac: Amann and Brosius (1985). Gene 40,183), pET-3 (T7: Studier and Moffat (1986) J. Mol. Biol. 189,113), the pFLEX series of expression vectors (Pfizer Inc., CT, USA) or the pQE series of expression vectors (Qiagen, Calif.), amongst others.

Suitable promoters for use in eukaryotic expression vectors include those capable of regulating expression in mammalian cells, insect cells such as Sf9 or Sf21 (Spodoptera furgiperda) cells, yeast cells and fungal cells. Preferred promoters for expression in eukaryotic cells include the p10 promoter, MMTV promoter, polyhedron promoter, the SV40 early promoter and the cytomegalovirus (CMV-IE) promoter, promoters derived from immunoglobulin-producing cells (see, U.S. Pat. No. 4,663,281), polyoma virus promoters, and the LTR from various retroviruses (such as murine leukemia virus, murine or Rous sarcoma virus and HIV), amongst others (See, Enhancers and Eukaryotic Gene Expression, Cold Spring Harbor Press, New York, 1983, which is incorporated herein by reference). Examples of other expression control sequences are enhancers or promoters derived from viruses, such as SV40, Adenovirus, Bovine Papilloma Virus, and the like.

A preferred expressible format for the modified ABC transporter polypeptide of the invention is achieved by placing the nucleotide sequence encoding said polypeptide and a promoter to which it is operably connected within a gene expression construct or vector.

Accordingly, a further aspect of the present invention provides a gene construct comprising a nucleotide sequence encoding the modified ABC transporter polypeptide of the invention.

The gene construct is preferably a plasmid or a retrovirus vector. Numerous expression vectors suitable for the present purpose have been described and are readily available. The expression vector may be based upon the pcDNA3 vector (Medos Company Pty Ltd, Victoria, Australia) that comprises the CMV promoter and BGH terminator sequences. Alternatively, the SG5 expression vector (Greene et al. (1988) Nucleic Acids Res. 15, 369; Stratagene), or the pQE series of vectors (Qiagen) are particularly useful for such purposes.

A preferred mammalian plasmid-based gene expression construct is the pRc/CMV plasmid (Invitrogen), which utilizes the CMV promoter to drive expression in mammalian host cells. Alternatively, a retroviral expression vector containing the Harvey murine sarcoma virus (Ha-MSV) long terminal repeats (LTRs) flanking the promoter and nucleic acid encoding the modified ABC transporter polypeptide may be used. One preferred Ha-MSV is the pC01 expression vector.

The gene constructs described herein may further comprise genetic sequences corresponding to a bacterial origin of replication and/or a selectable marker gene suitable for the maintenance and replication of said gene construct in a prokaryotic or eukaryotic cell, tissue or organism. Such sequences are well known in the art.

Selectable marker genes include genes which when expressed are capable of conferring resistance on a cell to a compound which would, absent expression of said selectable marker gene, prevent or slow cell proliferation or result in cell death. Those skilled in the art are aware that various antibiotic-resistance genes, such as those conferring resistance to ampicillin, Claforan, gentamycin, G418, hygromycin, rifampicin, kanamycin, neomycin, spectinomycin, or tetracycline, are generally used in such gene constructs as selectable markers.

The origin of replication and/or a selectable marker gene is preferably separated from the coding sequences that encode the modified ABC transporter polypeptide.

Methods for the production of recombinant plasmids, cosmids, bacteriophage molecules or other recombinant molecules are well known to those of ordinary skill in the art and can be accomplished without undue experimentation.

The gene constructs of the invention, including any expression vectors, are capable of introduction into, and expression in, an in vitro cell culture, or for introduction into, with or without integration into the genome of a cultured cell, cell line and/or transgenic animal.

In a particularly preferred embodiment contemplated herein, the gene constructs are used in gene therapy to transfer nucleic acid encoding the modified ABC transporter polypeptide to human cells. Preferably, such transfer is for the purposes of transplanting human cells expressing the modified ABC transporter polypeptide to humans during somatic therapy. Gene delivery systems may be viral, such as, for example, using retrovirus-based vectors or Adenovirus-based, or alternatively, a non-viral delivery system may be used, including any plasmid DNA-based delivery systems. For example, human haemopoietic cells or bone marrow cells or cells of the gastrointestinal tract are transfected with Ad21 or other adenovirus expressing the modified ABC transporter of the invention, and the transfected cells transplanted into the appropriate organ of a human patient to enhance drug resistance in that organ. Methods for performing somatic gene therapy are known to those skilled in the art (Fibison (2000) Nurs. Clin. North Am. 35, 757-772).

The present invention also provides a transformed cell comprising the nucleic acid molecule of the invention.

As used herein, unless the context requires otherwise, the word “cell” shall be taken to include a clonal or non-clonal group of cells. A group of cells may be functionally organized into whole tissue, an organ, or organism, or into a part of said tissue, organ or organism. The term “cell” shall further include any cell lysate of an isolated cell or group of cells.

As used herein, the term “transformed cell” is meant to also include the progeny of a transformed cell.

The host cell may be a mammalian cell, more preferably a human cell, canine cell, rat cell, rabbit cell or murine cell, and even more preferably the cell is a drug-sensitive primary epithelial cell or non-epithelial cell of humans, such as, for example, a bone marrow cell, a cell of the gastrointestinal tract, or a cell of the haematopoietic system.

Examples of eukaryotic cell lines contemplated herein to be useful include NIH 3T3, COS, VERO, HeLa, mouse C127, mouse L1210, Chinese hamster ovary (CHO), WI-38, baby hamster kidney (BHK), and MDCK cell lines. Such cell lines are readily available to those skilled in the art.

In a particularly preferred embodiment, the host cell is a non-polarized, such as, for example, the murine leukaemia cell line L1210, or alternatively, a polarized cell, such as an MDCK cell.

Means for introducing the isolated nucleic acid molecule or a genetic construct comprising same into a cell for expression of the immunogenic component of the vaccine composition are well known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and micropartide bombardment such as by using DNA-coated tungsten or gold particles (Agracetus Inc., WI, USA).

Moreover, transfection of a mammalian cell with the gene construct of the present invention results in the transformation of polarized and non-polarized cells from a drug-sensitive phenotype to a drug-resistant phenotype. Thus, cells that would normally be damaged or killed by certain drugs, such as chemotherapeutic agents (e.g. busulfan), are able to tolerate much higher levels of drug exposure with little or no adverse effect The cells thus acquire a multidrug resistant phenotype comparable to that observed in tumor cells subjected to various chemotherapeutic agents.

Accordingly, the gene construct according to this embodiment of the invention is particularly useful for conferring novel drug resistance characteristics on a cell, in particular a non-polarized cell, or alternatively, for transporting particular drugs from the cell.

Wherein the cell is a non-polarized cell, such as, for example, certain non-epithelial cells including fibroblasts and cells of the haemopoietic system, the modified ABC transporter polypeptide is localized generally within the plasma membrane. This confers resistance on the non-polarized cell, which would otherwise have a reduced efflux capacity.

Alternatively, wherein the cell is a polarized cell, such as, for example, a cultured epithelial cell (e.g. MDCK, Caco-2) or a primary epithelial cell (e.g. hepatocytes, intestinal cell, hippocampal neurons), the modified ABC transporter polypeptide is surprisingly distributed predominantly to the basolateral membrane. Localization of the modified ABC transporter polypeptide to the basolateral membranes of a polarized cell facilitates the efflux of certain ligand drugs from the cell via the basolateral membrane to confer resistance properties thereon.

It is highly desirable for the expressed modified ABC transporter polypeptide to confer resistance on the cell to one or more chemical compounds, such as, for example, a cytostatic or cytotoxic compound used in the treatment of infection or disease. For example, protection of non-polarized cells (e.g. cells of the haematopoietic system) is desirable during the treatment of patients with cytotoxic or cytostatic compounds.

As used herein, the term “chemical compound” shall be taken to mean any natural product, or synthetic compound having a definable chemical structure, and, in particular, a natural product or synthetic compound that is capable of being actively-transported into or out of a cell. Those skilled in the art will be aware that active-transport refers to an energy-dependent transport process, such as, for example, a transport process utilizing ATP or GTP or a nucleoside analogue thereof. Preferably, the chemical compounds against which resistance or sensitivity is modulated in accordance with the invention are those chemical compounds that are transported via ABC transporters, membrane transporters, or like transporters.

More preferably, the chemical compounds against which resistance or sensitivity is modulated in accordance with the invention are natural products or synthetic compounds. These are also useful in the treatment and/or prophylaxis of a disease of humans or other animals, such as, for example, anti-bacterial, anti-fungal, and, more preferably, chemotherapeutic agents.

Preferred anti-bacterial agents are antibiotic compounds. Antibiotics include quinolone antibiotics, sulfonamide antibiotics, cephalosporin antibiotics, or aminoglycoside antibiotics. these may be selected from the group consisting of acyclovir, adriamycin, antimycin, amikacin, amoxicillin, amoxicillin/clavulanate (augmentin), amphotericin b (fungizone), ampicillin, atovaquone (mepron), azithromycin (zithromax), cefazolin, cefepime (maxipime), ceftazidime, cefotaxime (claforan), cefotetan (cefotan), cefpodoxime (vantin), ceftizoxime (cefizox), ceftriaxone (rocephin), cefuroxime (zinacef), cephalexin, clotrimazole (mycelex), ciprofloxacin (cipro), clarithromycin (biaxin), clindamycin (cleocin), oxycycline, erythromycin lactobionate, famciclovir (famvir), fluconazole (diflucan), foscamet (foscavir), ganciclovir, gentamicin, imipenem/cilastatin (primaxin), isoniazid, itraconazole (sporanox), nafcillin, nitrofurantoin, nystatin, oligomycin, paromomycin, penicillin g, piperacillin/tazobactam (zosyn), rifampin (rifadin), ticarcillin/clavulanate (timentin), tobramycin, trimethoprim sulfamethoxazole, valacyclovir (valtrex), and vancomycin. The invention also extends to conferring resistance against the chloride salts or sulfated derivatives of the antibiotics supra, or against any derivative or related compound.

Preferred anti-fungal compounds are imidazoles (including bifonazole [i.e. 1-(α-biphenyl-4-ylbenzyl)imidazole], clotrimazole, intraconazole, fluconazole, econazole nitrate, ketoconazole, astemizole, metronidazole (flagyl) and miconazole nitrate [i.e. 1-[2,4-dichloro-β-(2,4-dichlorobenzyloxy)phenethyl]imidazole nitrate]); allylamines (including terbafine hydrochloride and terfenadine); amorolfine hydrochloride (cis-4-[(RS)3-[4(1,1-dimethylpropyl)phenyl]-2-methyl propyl]-2,6-dimethyl morpholine hydrochloride) and chloride salts and sulfated derivatives thereof, and derivatives and related compounds thereto.

A “chemotherapeutic agent” is a cytostatic and/or cytotoxic compound that is capable of rendering a mammalian cell inviable (i.e. a cytotoxin). A chemotherapeutic agent will at least reduce the capacity of a cell to grow and/or to proliferate (i.e. a cytostat). The cytotoxic or cytostatic properties of chemotherapeutic agents confer utility on these compounds in the therapeutic or prophylactic treatment of a cancerous or pre-cancerous cell, or a tumor, in an animal.

Preferred chemotherapeutic agents are selected from the group consisting of: busulphan (busulfan), cisplatin, cyclophosphamide, chlorambucil, BCNU, melphalan, merchlorethane, vinblastine sulphate, and etoposide (VP-16. VP-16-213, or VePesid). Other chemotherapeutic agents include vinca alkaloids selected from the group consisting of: vincristine sulfate, oncovin, velban, velsar, taxol, and epipodophyllotoxin (including podophyllotoxin and the synthetic derivatives thereof, teniposide (VM-26). The estrogen receptor antagonist tamoxifen, and the anti-neoplastic antibiotics adriamycin, bleomycin, doxorubicin, daunorubicin, daunomycin, rubidomycin, cerubidine, daunoblastina, plicomycin, and mitoxanthrone, and chloride salts and sulfated derivatives thereof, and related compounds thereto, are also useful in chemotherapy.

A further aspect of the invention provides a method of enhancing the resistance of a cell to a chemical compound comprising expressing a modified ABC transporter polypeptide in said cell for a time and under conditions sufficient for said cell to have modified growth and/or viability in the presence of said compound. Cell viability assays have been described in detail (Cui et al (1999) Mol. Pharmacol. 55, 929-937) and are readily adapatable to determining the enhanced resistance of cells expressing the modified ABC transporters of the invention.

This embodiment of the present invention clearly encompasses the conferring of enhanced growth and/or viability in the presence of the chemical compound or drug being tested. By virtue of its retained ability to enhance efflux of any substrate, in combination with its different membrane localization compared to the corresponding native ABC transporter, the modified ABC transporter of the invention enhances efflux of cytotoxic/cytostatic compounds compared to the corresponding native ABC transporter. Whilst not being bound by any theory or mode of action, the compound may be conjugated to glutathione, glucuronate, or sulfate, before it is transported from the cell.

For example, efflux of a cytotoxic/cytostatic drug substrate from a transfected polarized cell that expresses both the modified ABC transporter and the corresponding endogenous native ABC transporter will occur via both the apical and basolateral membranes, thereby enhancing total efflux compared to a non-transfected polarized cell.

Similarly, efflux of a cytotoxic/cytostatic drug substrate from a transfected non-polarized cell that expresses both the modified ABC transporter and the corresponding endogenous native ABC transporter will occur via the plasma membrane rather than being localized in the intracellular vesicles, thereby enhancing total efflux compared to a non-transfected non-polarized cell. As exemplified herein, ectopic expression of the modified cMOAT polypeptide of the invention enhances resistance of L1210 cells to Busulfan compared to non-transected L1210 cells.

The distribution pattern of naturally-occurring ABC transporter polypeptides in the tissues of humans or mammals provides for the extension of this aspect of the invention to further include the site-specific enhancement of drug resistance in humans and animals. According to this embodiment of the invention, the modified ABC transporter polypeptide of the invention is used in combination with one or more inhibitors of an ABC transporter which is different to that from which said modified ABC transporter polypeptide is derived (i.e. a heterologous ABC transporter polypeptide).

Accordingly, a further aspect of the invention provides a method of protecting a non-polarized cell of an organism or tissue comprising said non-polarized cell during the administration of a cytotoxic or cytostatic chemical compound to a subject, said method comprising:

(i) expressing a modified ABC transporter polypeptide in said non-polarized cell for a time and under conditions sufficient for said cell to efficiently transport said cytotoxic or cytostatic compound from said cell or otherwise acquire resistance to said compound; and

(ii) optionally, administering one or more inhibitors of an ABC transporter for a time and under conditions sufficient for ablating or inhibiting the growth of the cell expressing said ABC transporter, wherein said ABC transporter is different to that from which said modified ABC transporter polypeptide is derived (i.e. a heterologous ABC transporter polypeptide) and is involved in the transport of said cytotoxic or cytostatic chemical compound.

Preferably, the cell of sub-paragraph (ii) supra is a polarized cell or a non-polarized tumor cell. Preferably, the non-polarized cell of a sub-paragraph (i) supra is a cell of the haematopoietic system.

In a particularly preferred embodiment, the cytotoxic/cytostatic compound is a chemotherapeutic agent, such as, for example, Busulfan.

For example, modified cMOAT can be used to protect the haematopoietic system during chemotherapy that ablates non-haemopoietic tumor cells. Preferably during such therapeutic regimes, one or more P-gp antagonists can also be administered to inhibit P-gp activity in non-haemopoietic cells, to enhance the efficacy of the chemotherapeutic agent. Wherein P-gp activity is also inhibited, it is particularly preferred that such inhibition is in respect of endogenous P-gp activity in an epithelial tumor cell or alternatively, in a non-polarized tumor cell that over-expresses P-gp.

Similarly, a modified cMOAT polypeptide can be used to protect the haematopoietic system, preferably in conjunction with one or more MDR antagonists to inhibit MDR activity in the apical membrane of a non-hematological tumor cell, and one or more chemotherapeutic agents to inhibit tumorigenesis.

Similarly, a modified cMOAT polypeptide can be used to protect the haematopoietic system, preferably in conjunction with one or more antagonists to inhibit the activity of MRP1 and its homologues in the basolateral membrane of tumor cells, and one or more chemotherapeutic agents to ablate tumor cells.

In an alternative embodiment, a modified MDR3 polypeptide can be used to protect the haematopoietic system, preferably in conjunction with one or more cMOAT antagonists to inhibit cMOAT activity and/or one or more antagonists to inhibit MDR homologue activity in the membrane of non-hematological tumor cells and/or one or more antagonists to inhibit the activity of MRP1 and its homologues in the basolateral membrane of tumor cells, and one or more chemotherapeutic agents to ablate tumor cells.

In an alternative embodiment, a modified MDR homologue polypeptide can be used to protect the haematopoietic system, preferably in conjunction with one or more cMOAT antagonists to inhibit cMOAT activity and/or one or more P-gp antagonists to inhibit P-gp activity in the membrane of non-hematological tumor cells and/or one or more antagonists to inhibit the activity of MRP1 and its homologues in the basolateral membrane of tumor cells, and one or more chemotherapeutic agents to ablate tumor cells.

It will be apparent from the preceding description that a modified cMOAT polypeptide or modified MDR3 polypeptide or modified MRP4 polypeptide can be used to confer resistance in any non-polarized cell in which the corresponding naturally-occurring ABC transporter polypeptide is not present or active. In such embodiments it will also be apparent that the invention does not require simultaneous or consequential inhibition of endogenous ABC transporter activity in non-hematological tumor cells, notwithstanding that this feature is clearly encompassed by the invention.

The present invention further provides for the enhancement of drug resistance in a polarized cell in which the corresponding naturally-occurring ABC transporter polypeptide is already present or active in the apical membrane domain, preferably alongside the use of one or more ABC transporter antagonists to inhibit a heterologous ABC transporter polypeptide activity in tumorigenic non-polarized cells, and the use of one or more chemotherapeutic agents to ablate the tumor.

Accordingly, in an alternative embodiment, the present invention provides a method of enhancing the resistance of a polarized cell of an organism or tissue comprising said polarized cell during the administration of a cytotoxic or cytostatic chemical compound to a subject, said method comprising:

(i) introducing or expressing a modified ABC transporter polypeptide in said polarized cell for a time and under conditions sufficient for said cell to enhance transport said cytotoxic or cytostatic compound from said cell or otherwise enhance resistance to said compound; and

(ii) optionally, administering one or more inhibitors of an ABC transporter of a cell for a time and under conditions sufficient for ablating or inhibiting the growth of said cell, wherein said ABC transporter is different to that from which said modified ABC transporter polypeptide is derived (i.e. a heterologous ABC transporter polypeptide) and is involved in the transport of said cytotoxic or cytostatic chemical compound.

Preferably, the cell of subparagraph (ii) supra is a non-polarized cell. Preferably, the polarized cell is a primary epithelial cell (e.g. hepatocyte, intestinal cell, or hippocampal neuron, amongst others).

Preferred inhibitors of the cMOAT polypeptide and homologous polypeptides of other species are listed in Table 3.

TABLE 3
INHIBITORS OF cMOAT POLYPEPTIDES

Cholestatic agents:
α-Naphthylisothiocyanate, Chlorpromazine, Cyclosporin, Estradiol-17β-
glucuronide, Ethinylestradiol, Glycolithocholate-3α-O-sulfate,
Lithocholate-3α-O-glucuronide, Manganese-bilirubin, Phalloidin,
Taurocholate, Taurolithocholate

The present invention clearly contemplates the administration of a cytostatic compound or cytotoxic compound to a subject, wherein said compound exerts its effect on cells of both polarized and non-polarized lineage or type, with subsequent administration or co-administration or prior administration of the modified ABC transporter polypeptide of the invention to enhance resistance to said chemical compound in a sub-set of those cells. For example, the cytotoxic effects of a generally cytotoxic compound on the haematopoietic system of humans may be alleviated by subsequent administration, or co-administration, or prior administration, of the modified ABC transporter polypeptide of the invention to those haematopoietic cells, thereby enhancing their resistance to the compound. The benefits of such an approach are evident to those skilled in the art, particularly in so far as it relates to the application of cytotoxic and cytostatic compounds to cells, such as, for example, the chemotherapeutic treatment of cancers.

The present invention extends to the use of any and all modified ABC transporter polypeptides that are required for the influx/efflux of a chemical compound to enhance resistance of the cell to said chemical compound.

As the inventive method is broadly applicable to enhancing the drug resistance of any cell, the cell may be any polarized or non-polarized cell or cell line referred to herein above. Preferably, the cell is a non-cancerous cell or non-infected host cell of humans or other mammals. In a particularly preferred embodiment of the invention, the cell is a non-polarized cell, such as, for example a cell of the haematopoietic system.

The invention further extends to the use of any and all nucleic acid molecules that encode the modified ABC transporter polypeptides, to enhance the resistance of the cell to the said chemical compound. Preferably, this embodiment of the invention comprises the further step of introducing to the cell, tissue, organ or whole organism an isolated nucleic acid that encodes the modified ABC transporter polypeptide or functional variant of said polypeptide.

This embodiment further includes methods of in vivo gene therapy that produce the modified ABC transporter polypeptide de novo in the cell, tissue, organ or organism, using art-recognized procedures for gene therapy. For example, bone marrow can be transduced to have an altered expression of the modified ABC transporter polypeptide, thereby conferring resistance to chemotherapeutic drugs upon bone marrow cells. Following autologous transplantation of the transduced bone marrow, a more efficient chemotherapeutic regimen can be applied to cancer patients. The nucleic acid molecule used in performing this embodiment of the invention may be the exemplified nucleic acid described herein, or a homologue, analogue or derivative thereof encoding a modified ABC transporter polypeptide.

The gene therapy techniques described herein can also be used to ameliorate myelosuppression due to chemotherapy. In particular, the glutathione S-transferase isoenzymes having a synergistic effect with the glutathione conjugate transporters, such as, for example, cMOAT, decrease the cytotoxicity of chemotherapeutic agents. Accordingly, one or more vectors co-expressing the modified ABC transporter polypeptide of the invention and glutathione S-transferase are useful for increasing the efficiency of detoxification, such as by the liver. The co-expression of both the modified ABC transporter of the invention and glutathione S-transferase from the same or different vectors is clearly contemplated herein.

Such gene therapy techniques can also be used to treat liver dysfunction. Liver dysfunction can result from a genetic disease (Dubin Johnson's Syndrome) or due to lifestyle-influenced dysfunction resulting in cholestasis. The transplantation of non-polarized cells into liver is possible, but these cells do not normally integrate into the structures that form the canalicular spaces. Moreover, the ABC transporters that are normally distributed to the canalicular membrane of polarized cells are localized intracellularly in such non-polarized cells. Non-polarized cells that have been genetically transformed to express the modified ABC transporter polypeptide of the invention function to metabolize substrates and transport metabolites into the sinusoidal spaces which ultimately could be filtered by the kidneys.

Additionally, the modified ABC transporter polypeptide of the invention is used to develop novel cell lines for assaying ABC transporter activity, substrate specificity, or drug metabolism or drug transport. Clearly, cells expressing the modified ABC transporter of the invention are useful in this respect for determining the role of the transporter in the metabolism of any particular drug.

Accordingly, a further aspect of the invention contemplates a simple and reliable in vivo screening system for the discovery of novel agonists and antagonists of an ABC transporter polypeptide.

In particular, the present invention contemplates a simple and reliable in vivo screening system for discovery of novel agonists and antagonists of naturally-occurring ABC transporter polypeptides. The observation by the inventors that the modified ABC transporter polypeptide of the invention has the same function as the naturally-occurring counterpart, and is localized in the plasma membranes of both polarized and non-polarized cell types, indicates the utility of the invention in high throughput screening to identify agonists and antagonists of endogenous ABC transporter activities in these cells. The present invention clearly contemplates a process which utilizes rapid, high throughput screens with some tolerance of non-specificity and/or smaller-scale functional screens having higher specificity, and/or quantitative kinetic studies to elucidate chemical structure/function relationships to be determined, such as, for example, the elucidation of the docking site for agonist/antagonist molecules using the mutants of the modified proteins.

Preferably, the present invention contemplates a process for identifying a substrate of a native ABC transporter polypeptide comprising:

(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted;

(ii) determining the efflux of the compound from the cell expressing the modified ABC transporter relative to a cell that does not express the native ABC transporter or the corresponding modified ABC transporter, wherein efflux from the cell expressing the modified ABC transporter indicates that the compound is a substrate for the corresponding native ABC transporter.

Standard methods may be used to determine the efflux of the compound from the cell.

In a preferred embodiment, the present invention further provides a method for identifying an inhibitor of a native ABC transporter polypeptide comprising:

(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted;

(ii) incubating the cell in the presence of (a) a compound being tested for its ability to inhibit activity of the ABC transporter polypeptide; and (b) a known substrate compound for said native ABC transporter polypeptide;

(iii) in a separate sample to (ii), incubating the cell in the presence of said substrate compound; and

(iv) comparing the efflux of the substrate compound at (ii) and (iii), wherein reduced efflux at (ii) compared to (iii) indicates that the compound being tested is an inhibitor of the native ABC transporter polypeptide.

Based upon the similar activities of the modified ABC transporter polypeptide and the corresponding naturally-occurring ABC transporter polypeptide, it is preferred that the inhibitory compound identified in this assay is also an inhibitor of the corresponding naturally-occurring ABC transporter polypeptide.

It will be apparent to those skilled in the art that the assay format described herein is readily adapted to determine novel agonists of ABC transporter function. Accordingly, an alternative embodiment of this assay format provides a method for identifying an agonist of a native ABC transporter polypeptide comprising:

(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted;

(ii) incubating the cell in the presence of (a) a compound being tested for its ability to agonize activity of the ABC transporter polypeptide; and (b) a known substrate compound for said native ABC transporter polypeptide;

(iii) in a separate sample to (ii), incubating the cell in the presence of said substrate compound; and

(iv) comparing the efflux of the substrate compound at (ii) and (iii), wherein enhanced efflux at (ii) compared to (iii) indicates that the compound being tested is an agonist of the native ABC transporter polypeptide.

Based upon the similar activities of the modified ABC transporter polypeptide and the corresponding naturally-occurring ABC transporter polypeptide, it is preferred that the agonist identified in this assay is also an agonist of the corresponding naturally-occurring ABC transporter polypeptide.

In an alternative embodiment, agonists may be identified by a process comprising:

(i) expressing a modified ABC transporter polypeptide in the plasma membrane of a polarized or non-polarized cell;

(ii) incubating the cell in the presence of (a) a compound being tested for its ability to agonize activity of the ABC transporter polypeptide; and (b) a known substrate compound for said modified ABC transporter polypeptide;

(iii) in a separate sample to (ii), incubating an isogenic cell that does not express the modified ABC transporter polypeptide in the presence of said substrate compound and said compound being tested; and

(iv) comparing the efflux of the said substrate compound at (ii) and (iii), wherein enhanced efflux at (ii) compared to (iii) indicates that the compound being tested is an agonist of said modified ABC transporter polypeptide.

Preferably, the isogenic cell does not express any ABC transporter polypeptide capable of transporting the substrate compound used in the assay formats described herein.

Preferred substrates which are transported by MRP1, MRP2, and MRP3 are listed in Table 4. Substrates for these transporters generally have a lipophilic moiety, such as, for example, bilirubin, estradiol, or arachidonate, linkes to at least one anionic residue, such as, for example, glucuronosyl, carboxyl, glutathionyl, or sulfate. As will be known to those skilled in the art, a conjugated substrate, particularly a glutathione conjugate, can generally be provided to the cell in an unconjugated form wherein it is conjugated by the action of an endogenous enzyme, such as, for example, glutathione-S-transferase.

Preferred substrates of modified cMOAT include leukotriene C4 (LTC4; Du Pont); bilirubin; monoglucuronosyl bilirubin (Jedlitschsky et al (1997) Biochem J. 327, 305-310; Kamisako et al (1999) Hepatol. 30, 485-490); bisglucuronosyl bilirubin (Jedlitschsky et al (1997) Biochem J. 327, 305-310; Kamisako et al (1999) Hepatol. 30, 485-490); leukotriene D4 (LTD4); 1,3-chloro-2,4-dinitrobenzene; mono-chlorobimane (thiolyte, Calbiochem); 7-chloro-4-nitrobenz-2-oxa-1,3-diazole (Sigma); 17β-glucuronosyl estradiol (Du Pont); 3α-sulfatolithocholyl taurine; Fluo-3 (Nies et al (1998) Hepatol. 28,1332-1340); glutathione disulphide (Leier et al (1996) Biochem J., 314, 433-437), and p-aminohippurate (Leier et al (2000) in press). For transport assays, the use of the following radioligands is preferred: [³H]-LTC4 (DuPont), [³H] 7β-glucuronosyl estradiol (Du Pont), [³H]monoglucuronosyl bilirubin. The use of the fluorescent substrate Fluo-3 is also preferred. Other substrates that can be readily measured include the following compounds capable of forming glutathione conjugates: 1,3-chloro-2,4-dinitrobenzene; mono-chlorobimane (thiolyte, Calbiochem); and 7-chloro-4-nitrobenz-2-oxa-1,3-diazole (Sigma). Of these compounds, 1,3-chloro-2,4-dinitrobenzene is converted to DNP-SG; mono-chlorobimane (thiolyte, Calbiochem) is converted to Bimane-SG; and 7-chloro-4-nitrobenz-2-oxa-1,3-diazole (Sigma) is converted to 4-nitrophenyl-2-oxa-1,3-diazole-SG.

Substrates for modified MDR3 include digoxin, paclitaxel, verapamil, vinblastine, phosphatidylcholine, and short chain phosphatidylcholine analogues, and these are conveniently radiolabeled for transport assays. for example, [12α³H]digoxin is readily available from New England Nuclear Life Sciences; [³H]paclitaxel is readily available from Moravek Biochemical Inc, (La Bresa, Calif., USA); [α-³²P]8-azido-ATP and [α-³²P]ATP are readily available from ICN Biomedicals (Costa Mesa, Calif., USA). [³H]verapamil has also been described elsewhere as having utility in assaying for MDR3 transport (Doppenschmitt et al (1999) J Pharmacol Exp Ther 288, 348-357).

Substrates for modified MRP4 include an amphiphilic anion supra, a nucleoside analog, or cyclic nucleotide. Preferred substrates for transport assays include the following: azidothymidine monophosphate; 9-(2-phosphonylmethoxyethyl)adenine (i.e. PMEA) (Schuetz et al (1999) Nature Med 5, 1048-1051); 6-mercaptopurine; 1,3-chloro-2,4-dinitrobenzene; CAMP; cGMP; Sildenafil (Pfizer); Trequinsin (Sigma); and Zaprinast (Sigma). For transport assays, these substrates are conveniently provided as radiolabeled compounds.

TABLE 4
SUBSTRATES OF MAMMALIAN MRP POLYPEPTIDES

| Substrate | MRP1 | MRP2 | MRP3 |
|---|---|---|---|
| 17β-Glucuronosyl estradiol | ✓ | ✓ | ✓ |
| 3α-Sulfatolithocholyltaurine | ✓ | | |
| 5-Methyltetrahydrofolate | | ✓ | |
| 6α-Glucuronosyl hyodeoxycholate | ✓ | | |
| Aflatoxin B1 + reduced glutathione | ✓ | | |
| ampicillin | | ✓ | |
| Bile salt conjugates | | ✓ | |
| Bilirubin- | | | |
| monoglucuronosyl- | ✓ | ✓ | ✓ |
| bisglucuronosyl | ✓ | ✓ | |
| BQ123 | | ✓ | |
| BQ485 | | ✓ | |
| BQ518 | | ✓ | |
| bromosulfophthalein-glutathione | | ✓ | |
| carboxydichlorofluorescein | | ✓ | |
| ceftriaxone | | ✓ | |
| Chlorambucil- | ✓ | | |
| monochloro-monoglutathionyl- | ✓ | | |
| monohydroxy-monoglutathionyl- | ✓ | | |
| bisglutathionyl | ✓ | | |
| cholate 3-O-glucuronide | | ✓ | |
| conjugated bilirubin | | ✓ | |
| copper (acute i.v. administration) | | ✓ | |
| coproporphyrin I | | ✓ | |
| CPT11 carboxylate | | ✓ | |
| cysteinyl-leukotrienes | | ✓ | |
| Daunorubicin + reduced glutathione | ✓ | | |
| dibromosulfophthalein | | ✓ | |
| dinitrophenyl-glutathione | | ✓ | |
| Fluo-3 | ✓ | ✓ | |
| Folate | ✓ | | |
| gadolinium-ethoxybenzyl-DTPA | | ✓ | |
| Glucuronosyl E3040 | | ✓ | ✓ |
| Glucuronosyl etoposide | ✓ | | |
| Glucuronosyl grepafloxacin | | ✓ | |
| Glucuronosyl nafenopin | | ✓ | |
| Glucuronosyl SN38 carboxylate | | ✓ | |
| Glucuronosyl SN38 lactone | | ✓ | |
| glutathione GSH | | ✓ | |
| glutathione GSSG | | ✓ | |
| Glutathione disulfide | ✓ | ✓ | |
| glutathionyl-bromoisovalerylurea | | ✓ | |
| indocyanine green | | ✓ | |
| Leukotriene C₄ | ✓ | ✓ | ✓ |
| Leukotriene D₄ | ✓ | ✓ | |
| Leukotriene E₄ | ✓ | ✓ | |
| lithocholate 3-O-glucuronide | | ✓ | |
| manganese | | ✓ | |
| Methotrexate | ✓ | ✓ | ✓ |
| N-Acetyl-leukotriene E₄ | ✓ | ✓ | |
| Melphalan- | | | |
| monochloro-monoglutathionyl- | ✓ | | |
| monohydroxy-monoglutathionyl | ✓ | | |
| nordeoxycholate 3-O-glucuronide | | ✓ | |
| nordeoxycholate 3-sulfate | | ✓ | |
| Ochratoxin A | | ✓ | |
| p-Aminohippurate | ✓ | ✓ | |
| Pravastatin | | ✓ | |
| S-Glutathionyl 2,4-dinitrobenzene (DNP-SG) | ✓ | ✓ | ✓ |
| S-Glutathionyl aflatoxin B1 | ✓ | | |
| S-Glutathionyl ethacrynic acid | ✓ | ✓ | |
| S-Glutathionyl N-ethylmaleimide | ✓ | | |
| S-Glutathionyl prostaglandin A₂ | ✓ | | |
| S-Glutathionyl sulfobromophthalein | | ✓ | |
| SN38 carboxylate | | ✓ | |
| Sulfobromophthalein | | ✓ | |
| tauro/glycolithocholate 3-sulfate | | ✓ | |
| taurochenodexoycholate 3-sulfate | | ✓ | |
| Temocaprilat | | ✓ | |
| triiodothyronine-glucuronide | | ✓ | |
| Vincristine + reduced glutathione | ✓ | | |
| zinc | | ✓ | |

In accordance with the preceding embodiments, the known substrate compound used in these assays may be a cytostatic compound or cytotoxic compound, such as, for example, any one or more of the various antibiotics, or chemotherapeutic agents that are normally transcytosed via an ABC transporter polypeptide from which the modified ABC transporter polypeptide employed in the assay is derived. In adapting the assays to employ such cytotoxic or cytostatic compounds, enhanced or reduced efflux may be estimated by the enhanced viability and/or growth or reduced viability and/or growth, respectively, of the cell. This is because any enhanced efflux of the cytotoxin or cytostatic compound due to the presence of an agonist of the modified ABC transporter polypeptide will generally enhance cell viability and/or growth, under appropriate conditions. Similarly, any reduced efflux due to the presence of an antagonist compound will have the effect of reducing cell survival and/or growth at appropriate concentrations of cytotoxin or cytostatic compound.

Preferably, the known substrate compound is capable of forming a conjugate with glutathione, glucuronate, or sulfate. For example, 1-chlroro-2,4-dinitrobenzene is conjugated with glutathione to form 2,4-dinitrophenylglutathione (DNP-GS). Similarly, mono-chlorobimane (thiolyte, Calbiochem) forms the glutathione conjugate bimane-glutathione. Similarly, 7-chloro-4-nitrobenz-2-oxa-1,3-diazole (Sigma) is conjugated to glutathione in the cell to form 4-nitrophenyl-2-oxa-1,3-diazole glutathione. In this assay format, efflux is conveniently determined by the appearance of these substrate compounds in the media. For example, cells expressing a modified ABC transporter are exposed briefly to 1 chloro-2,4-dinitrobenzene (CDNB), then washed and incubated with the putative agonists or antagonists being tested. After incubation, the supernatant is checked by spectrophotometry for the presence of 2,4-dinitrophenyl glutathione, and its rate of appearance is a measure of the activity of the agonist or antagonist compound. This process is readily configured to a high throughput mode (Board, P. (1984) FEBS Lett 124, 153-165; Olive and Board (1994) Biochem. Biophys. Acta 1224, 264-268).

the assay format used may be any convenient format for assaying transport, including a nucleotide trapping assay, or the use of cell monolayers. Such formats are known to those skilled in the art.

In the foregoing embodiment, the use of non-polarized cells is preferred, because they do not normally express the native counterpart of the modified ABC transporter polypeptide in their plasma membranes. However, polarized cells may also be used, because the modified ABC transporter polypeptide accumulates over a greater surface area of the plasma membrane compared to the endogenous ABC transporter polypeptide, which is localized in the apical membrane domain. When polarized cells are used to conduct these assays, the efflux of the cytotoxin or cytostat from the cell via the modified ABC transporter polypeptide is several fold (at least about 2-fold, preferably, at least about 5- to 7-fold) the level of efflux via any endogenous naturally-occurring ABC transporter polypeptide in the plasma membrane of the polarized cell.

Compounds detected using this screening procedure can ultimately be used, for example, as chemosensitizers in cancer therapy.

A further aspect of the invention contemplates the use of a T-K-F motif as a portable transport signal peptide for targeting proteins to the apical membrane subject to the proviso that the T-K-F motif is within the context of an ABC transporter polypeptide.

This invention is also described with reference to the following non-limiting examples.

### EXAMPLE 1

### Modified cMOAT (MRP2) Polypeptides

Introduction

cMOAT is an ABC transporter of the subfamily known in the art as multidrug resistance-associated proteins (MRPs). Several reviews of MRPs have been published (see Borst et al., (2000) J. Natl. Cancer Inst. 92,1295-1302; and Konig et al., (1999) Biochim. Biophys. Acta 1461, 377-394). There are six known human MRPs, designated MRP1 through MRP6. cMOAT corresponds to MRP2. The MRPs export a broad range of compounds from the cell. MRP1 was the first and most extensively characterized member (Cole et al., (1992) Science 258,1650-1654) and has 49% sequence identity with cMOAT (Buchler et al., (1996) J. Biol. Chem. 271, 15091-15098; Ito et al., (1997) Am. J. Physiol. 272, G16-G22; Paulusma et al., (1996) Science 271,1126-1128; and Taniguchi et al., (1996) Cancer Res. 56, 4124-4129).

MRP1 and cMOAT have similar substrates, which include glutathione conjugates, glucuronide conjugates, reduced glutathione, and chemotherapeutic drugs. The function of cMOAT was initially shown to be distinct from MRP1 by the use of cMOAT-deficient rats GY/TR2 (Jansen et al., (1985) Hepatology 5, 573-579; Jansen et al., (1987) Hepatology 7, 71-76; and Kitamura et al., (1990) Proc. Natl. Acad. Sci. U.S. A 87, 3557-3561) and EHBR (Hosokawa et al., (1992) Lab. Anim. Sci. 42, 27-34).

The distribution patterns of MRP1 and cMOAT also differs. MRP1 is found throughout the body in many tissues, including the haematopoietic system, the blood brain barrier, lungs, and at lower expression levels in the liver and kidneys. In contrast, cMOAT is only found at significant levels in the liver and to a lesser extent in the kidneys. In these two tissues, where both proteins are expressed, they differ in their specific cellular localization. MRP1 is found in the basolateral (sinusoidal) membrane and thus may serve to redirect potential excretion products back into the bloodstream. Conversely, cMOAT is solely found in the apical membrane, and this defines its function as an export pump of compounds destined for terminal excretion from the body. Although both proteins can be found in the hepatocyte, higher expression levels of cMOAT than MRP1 create the vectorial transport of excretion products from the blood into bile.

In initial experiments, haematopoietic cell lines transfected with cMOAT did not express a functional cMOAT due to intracellular accumulation of the protein and minimal cell membrane localization. Similar results have since been reported by others (see Evers et al., (1998) J. Clin. Invest 101, 1310-1319). In contrast, MRP1 shows total cell membrane localization in similarly transfected cells.

We sought to establish the sorting signals responsible for the difference in localization of MRP1 and cMOAT in both epithelial cell lines and cells of haematopoietic lineage. Using green fluorescent protein (GFP) fusion proteins and site-directed mutagenesis we identified a sequence motif responsible for exclusive apical localization of cMOAT in polarized MDCK cells. Deletion of the motif results in lateral localization of cMOAT in polarized MDCK cells and allows cell membrane localization in L1210 cells. The mutated protein transports 2,4-dinitro-phenylglutathione (DNP-GS), a known substrate of cMOAT produced by conjugation of 1-chloro-2,4-dinitrobenzene with glutathione.

Experimental Procedures

Green Fluorescent Protein (GFP) Fusion Protein Encoding Gene Constructs

GFP was fused to the C-terminal region of MRP1 or cMOAT polypeptides, to facilitate detection of the localization of the MRP1-gfp or cMOAT-gfp fusion proteins, as described below.

Human cMOAT cDNA was amplified by polymerase chain reaction using PfuTurbo DNA polymerase (Stratagene) to remove the stop codon and introduce restriction enzyme sites suitable for cloning. The cDNA was amplified using a sense primer that adds an NheI site immediately adjacent to the start codon, as follows:

(SEQ ID NO: 24)

5′-AGCGCTAGCGATGCTGGAGAAGTTCTGCAAC-3′;

and an antisense primer that adds an AgeI site after the final codon and removes the stop codon, as follows:

(SEQ ID NO: 25)

5′-TACGGTACCGGTGCGAATTTTGTGCTGTTCACATTC-3′.

The polymerase chain reaction product was digested with NheI/AgeI and ligated into the NheI/AgeI-digested EGFP-N1 vector (CLONTECH).

Additionally, human MRP1 cDNA was cloned from HL60ADR cells and ligated into EGFP-N1 (SaclI/AgeI) using the same polymerase chain reaction method as described in the preceding paragraphs, however employing different amplification primers. The MRP1 sense primer used, which introduces a SaclI site immediately adjacent to the start codon, was as follows:

(SEQ ID NO: 34)

5′-GCGGCCGCGGATGGCGCTCCGGGGCTTC-3′.

The antisense primer, which adds an AgeI site and removes the stop codon of MRP1, was as follows:

(SEQ ID NO: 35)

5′-TACGGTACCGGTGCCACCMGCCGGCGTCTTTGG-3′

The cMOAT-gfp and MRP1-gfp constructs supra (1 μg of DNA per transfection) were separately transfected into MDCK cells and L1210 cells using a UpofectAMINE transfection kit (Life Technologies, Inc.). Transfections of MDCK cells were carried out using Transwell plates (Costar, 24 mm×3 μm polycarbonate membrane) to enable cell polarization. Cells were imaged using a NikonTE300 inverted microscope linked to a Radiance 2000 Laser Scanning System for confocal microscopy and Lasersharp 2000 imaging software (Bio-Rad).

Expression of a cMOAT Polypeptide Lacking the T-K-F motif (ΔcMOAT)

A modified cMOAT nucleotide sequence encoding a modified cMOAT polypeptide wherein the C-terminal T-K-F motif was deleted (herein “ΔcMOAT”), and without a GFP tag, was prepared using the QuikChange site-directed mutagenesis kit. To produce this construct, template DNA comprising the cMOAT cDNA in the mammalian expression vector pRc/CMV (Invitrogen) (Taniguchi et al., (1996) Cancer Res. 56, 4124-4129) was amplified using a sense primer (SEQ ID NO: 26; Table 5) and antisense primer as follows:

| | |
|---|---|
| 5′-GGCCTTCTGCTAGCTGTTCACATTC-3′, | (SEQ ID NO: 36) |

thereby producing DNA wherein the nine nucleotides that encode amino acid residues Thr1543, Lys1544, and Phe1545 of native cMOAT were deleted.

Successful mutagenesis of two clones from separate reactions was confirmed by sequencing. Stable transfectants in L1210 cells were selected for further study.

Site-Directed Mutagenesis

Substitution mutations of cMOAT were achieved using the Quikchange Site-Directed Mutagenesis Kit (Stratagene). A double-stranded plasmid vector containing the wild-type cMOAT cDNA was used as a template to amplify mutant sequences, using batches of synthetic complementary oligonucleotides (Table 5) containing the desired mutations, which primers annealed to the 3′-end of the coding region of the cMOAT cDNA and were extended in a rolling circle amplification reaction catalyzed by PfuTurbo DNA polymerase enzyme. The annealing and extension temperatures used were as recommended by the manufacturer. In particular, we used 18 extension cycles for 19 minutes each, to amplify from 5-10 ng of template DNA in each case.

The primer sequences were thus incorporated into mutated plasmids containing staggered nicks. Following temperature cycling, the product was treated with the endonuclease DpnI, to digest only the template DNA containing methylated and hemi-methylated sequences. The nicked vector mutant DNA was then transformed into E. coli strain XL-1 blue (Stratagene), to repair the nick and replicate the mutated DNA sequences. E. coli cells transformed with each of the mutated plasmids was selected on kanamycin-containing plates. Colonies were cultured and DNA was isolated therefrom, and the mutations were confirmed by nucleotide sequence analysis of the recovered plasmids.

The sequences of the forward primers used in the site-directed mutagenesis of the nine nucleotides encoding the amino acid sequence of the C-terminal regions of several modified cMOAT polypeptides are listed in Table 5. Amino acid residues in bold type are those introduced by the site-directed mutagenesis. The complementary nucleotide sequences of the reverse primers are readily derived.

TABLE 5

| Protein | Sequence type | | Relevant Sequence |
|---|---|---|---|
| cMOAT | C-terminus | (SEQ ID NO: 37) | Asn Val Asn Ser Thr Lys Phe |
| | Primer | (SEQ ID NO: 26) | G AAT GTG AAC AGC ACA AAA TTC GCC |
| T1543A | C-terminus | (SEQ ID NO: 38) | Asn Val Asn Ser Ala Pro Val |
| K1544P | Primer | (SEQ ID NO: 27) | G AAT GTG AAC AGC GCA CCG GTC GCC |
| F1545V | | | |
| S1542A | C-terminus | (SEQ ID NO: 39) | Asn Val Asn Ala Thr Lys Phe |
| | Primer | (SEQ ID NO: 28) | G AAT GTG AAC GCC ACA AAA TTC GC |
| T1543A | C-terminus | (SEQ ID NO: 40) | Val Asn Ser Ala Lys Phe |
| | Primer | (SEQ ID NO: 29) | T GTG AAC AGC GCA AAA TTC GCACC |
| K1544A | C-terminus | (SEQ ID NO: 41) | Val Asn Ser Thr Ala Phe |
| | Primer | (SEQ ID NO: 30) | GTG AAC AGC ACA GCA TTC GCACCG |
| F1545A | C-terminus | (SEQ ID NO: 42) | Ser Thr Lys Ala |
| | Primer | (SEQ ID NO: 31) | C AGC ACA AAA GCC GCACCGGTCG |
| T1543A | C-terminus | (SEQ ID NO: 43) | Val Asn Ser Ala Ala Ala |
| K1544A | Primer | (SEQ ID NO: 32) | AT GTG AAC AGC GCA GCA GCC GCACCGGTCC |
| F1545A | | | |
| ΔT1543 | C-terminus | (SEQ ID NO: 44) | Asn Val Asn Ser * |
| ΔK1544 | Primer | (SEQ ID NO: 33) | G AAT GTG AAC AGC TAGCAGAAGGCC |
| ΔF1545 | | | |

2,4-Dinitrophenyl Glutathione (DNP-GS) Transport

DNP-GS was generated in L1210 cells by exposure to 1-chloro-2,4-dinitrobenzene and its efflux determined as described previously (Olive et al., (1994) Biochim. Biophys. Acta 1224, 264-268).

Immunofluorescence

Detection and localization of untagged mutant cMOAT lacking the T-K-F motif (i.e. ΔcMOAT) was achieved by immunofluorescence, using the antibody M2 III6 (Kamiya Pty Ltd). 2×10⁵ cells were washed with PBSF (phosphate-buffered saline supplemented with 2.5% fetal bovine serum). The cells were permeabilized using digitonin (5 μg/ml) and incubated at room temperature for 15 min. The cells were then washed three times with PBSF and then incubated with the primary antibody (2 μg) for 1 hr at room temperature before being washed twice with PBSF. The cells were incubated with fluorescein isothiocyanate-conjugated F(ab′)2 (Silenus, Hawthorn, Victoria, Australia) (1:80 dilution) for 30 min at room temperature. Finally, the cells were washed three times and resuspended in PBSF ready for immediate confocal microscopy.

Detection of P-glycoprotein was achieved using the antibody MRK16 (Kamiya Pty Ltd.). 2×10⁵ cells were washed with PBSF and incubated with the primary antibody (2 μg) for 1 h at room temperature then washed two times with PBSF. The cells were incubated with fluorescein isothiocyanate-conjugated F(ab′) (1:400 dilution) for 30 min, washed three times, and resuspended in PBSF ready for immediate confocal microscopy.

Results

Localization of cMOAT gfp and MRP1-gfp Fusion Proteins in MDCK Cells

To conveniently detect the localization of the proteins under investigation, GFP fusion proteins were produced and their localization visualized using confocal microscopy to visualize the fluorescent product, as described supra.

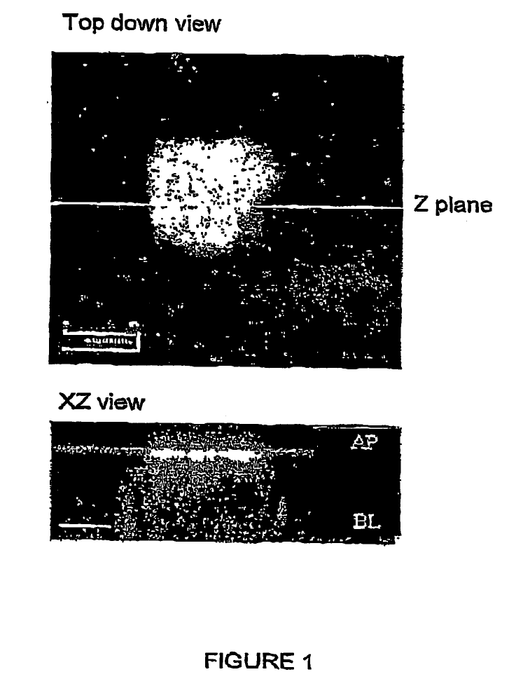

Using anti-cMOAT specific antibody, native cMOAT was previously shown to localize to the apical membrane of MDCK cells (Evers et al., (1998) J. Clin. Invest. 101, 1310-1319; and Cul et al., (1999)Mol. Pharmacol. 55, 929-937). In the present study, human cMOAT with GFP fused to its C terminus localized to the apical membrane in polarized MDCK cells, consistent with the localization of the native protein (FIG. 1). The apical membrane of polarized MDCK cells grown on Transwell membranes is the surface facing the media as opposed to the surface adhering to the membrane (basolateral).

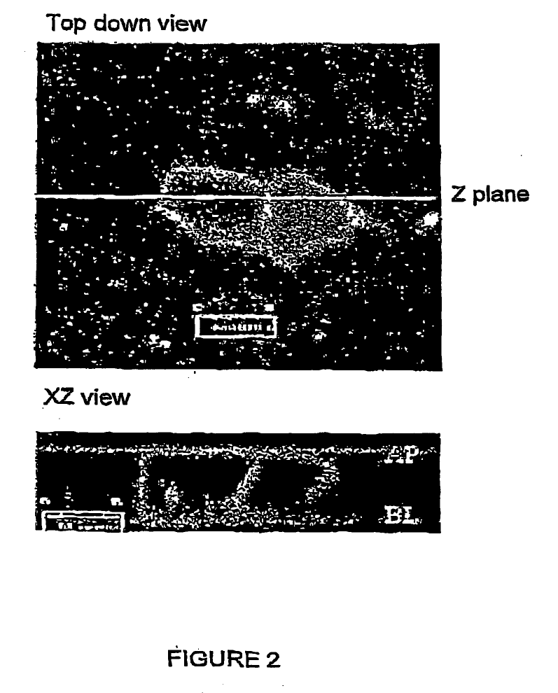

MRP1 has been previously immune localized to the basolateral membrane of a pig kidney epithelial cell line (LLC-PK1) (Evers et al., (1996) J. Clin. Invest. 97,1211-1218). In the present study, human MRP1 with GFP fused to its C terminus also demonstrated basolateral localization in polarized MDCK cells (FIG. 2).

These studies establish that fusion of MRP1 and cMOAT to GFP does not interfere with the normal targeting of these proteins to the basolateral and apical membranes.

Expression of ΔcMOAT in MDCK Cells

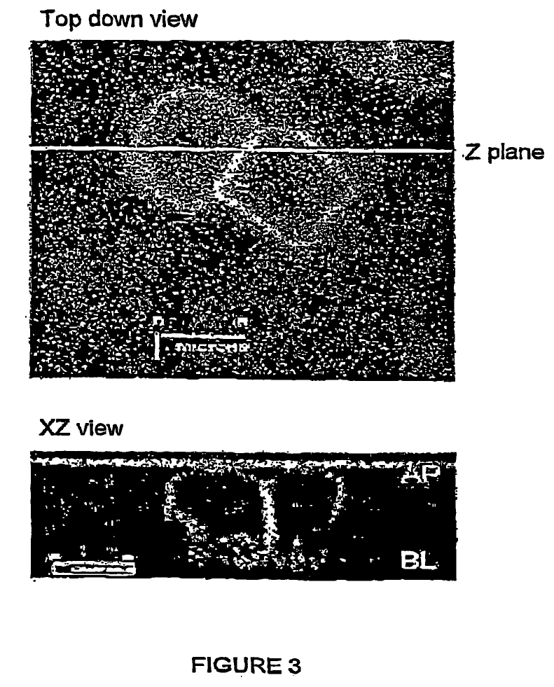

The alignment of C-terminal sequences of MRP1 and cMOAT revealed a C-terminal motif in cMOAT (herein “T-K-F motif”) that was absent in MRP1. To determine whether the TKF motif influenced the apical localization of cMOAT, we constructed ΔcMOAT-gfp in which the three C-terminal residues were deleted. When expressed in polarized MDCK cells, ΔcMOAT-gfp was found to localize predominantly in the lateral and/or basolateral membranes in contrast to the apical localization of cMOAT-gfp (FIG. 3).

Localization of cMOAT-gfp Substitution Mutants in MDCK Cells

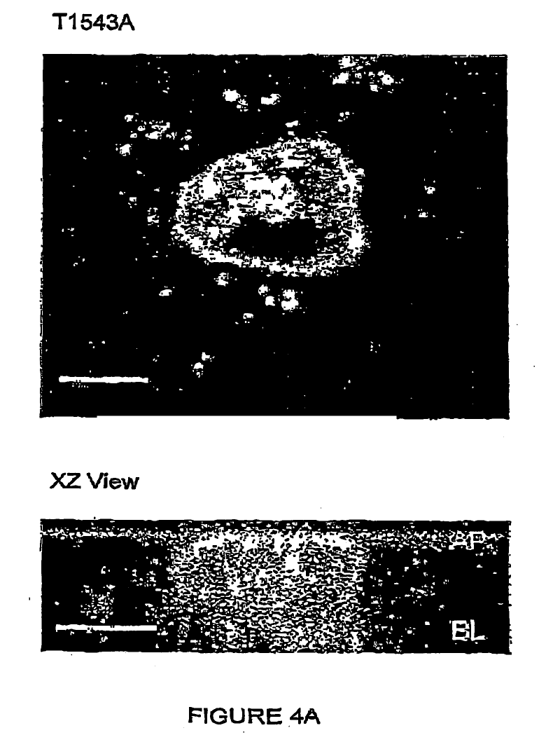

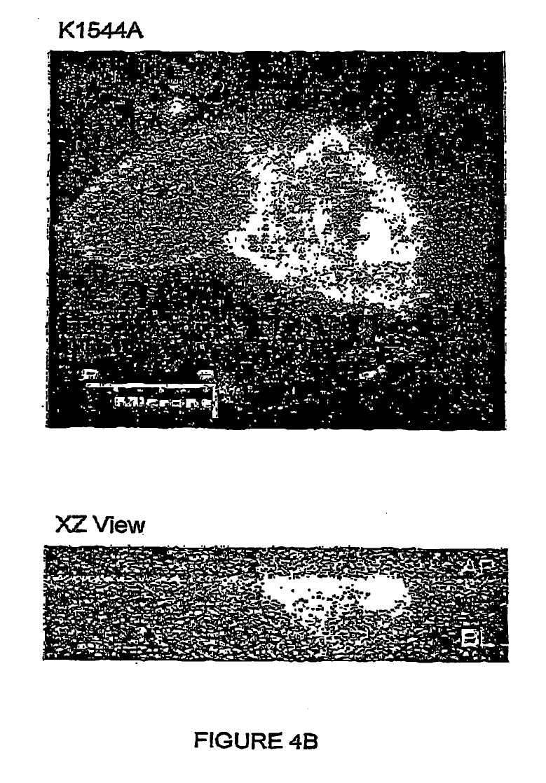

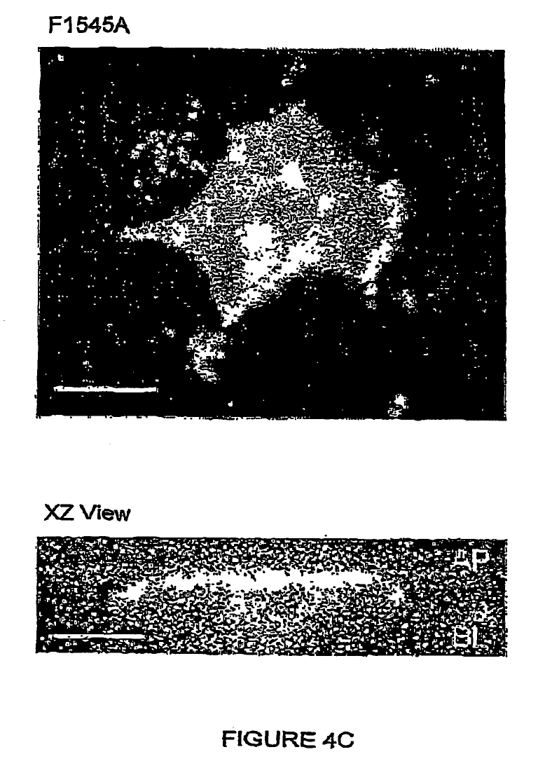

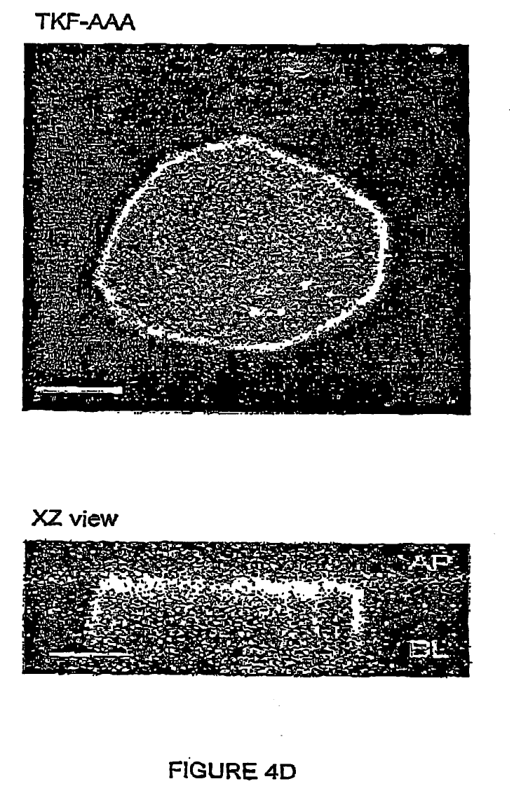

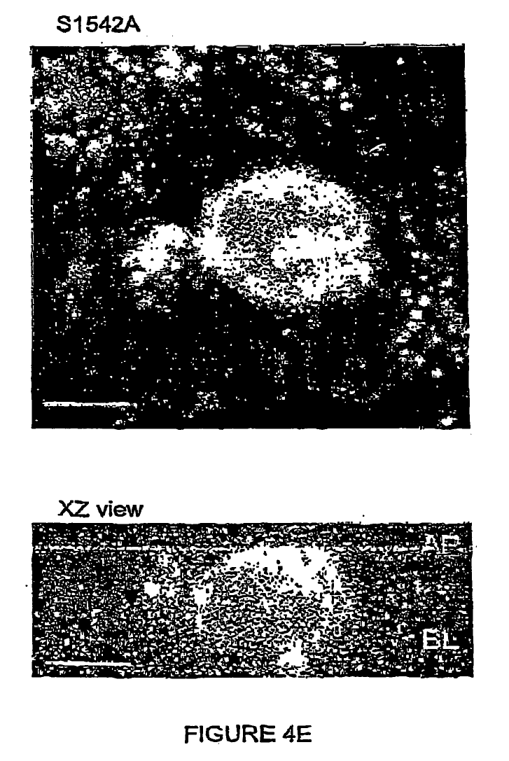

To further characterize the TKF motif of ΔcMOAT-gfp, and to determine the relative importance of each residue, individual alanine mutations were introduced into each of the residue positions 1543-1545 of the cMOAT-gfp construct (Table 5). Since residues 1542-1544 (S-T-K) form a predicted phosphorylation site, residue 1542 was also mutated to alanine. FIGS. 4A through 4E show the localization of each of these mutants in MDCK cells. The effects of the substitutions were determined by visualizing the change in localization of the mutant compared with the native protein. The T1543A and K1544A mutants (Table 5) exhibited both apical and basolateral targeting with an increase in protein accumulation in intracellular vesicles. The F1545A mutant (Table 5) did not exhibit modified localization in MDCK cells compared to native cMOAT. Mutation of all three residues to alanine (i.e. the T1543A K1544A F1545A mutant in Table 5) also caused the protein to be localized to the basolateral membrane.

Expression of cMOAT in L1210 Cells

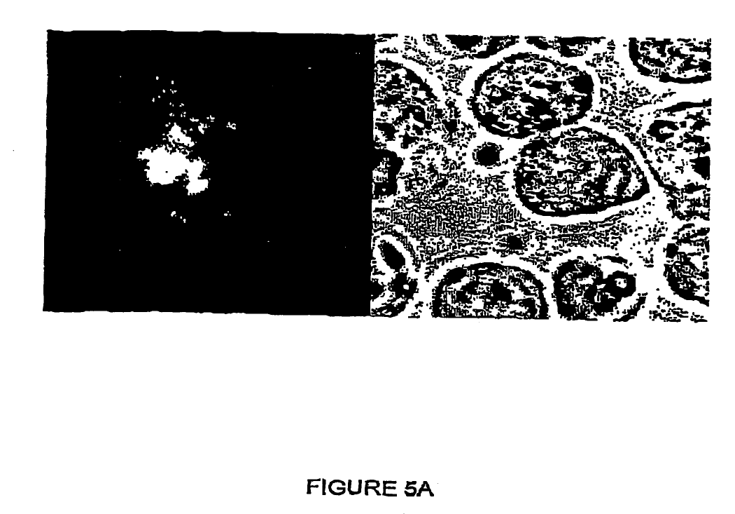

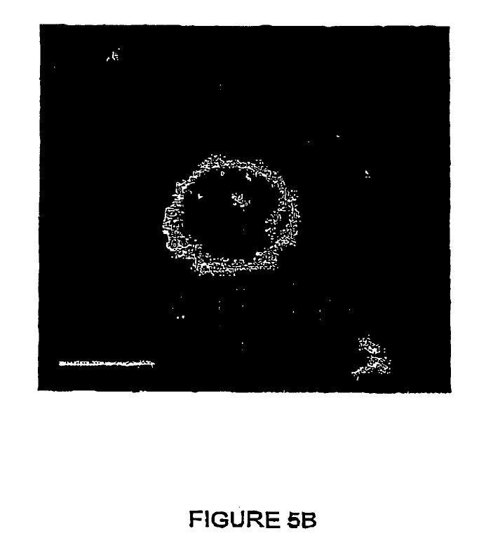

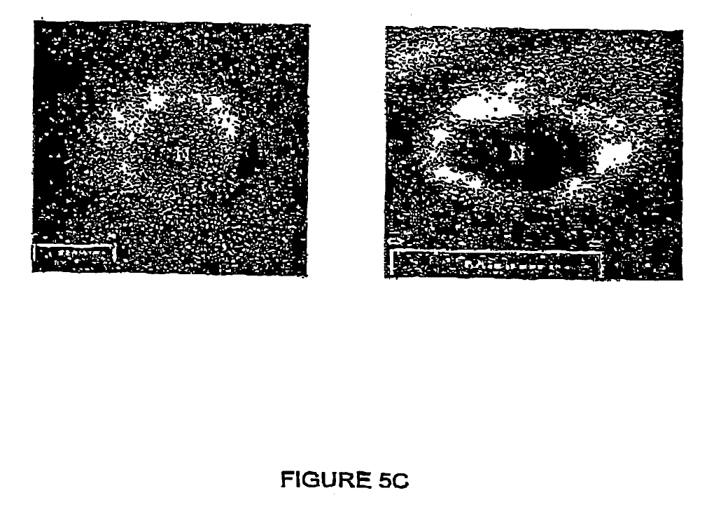

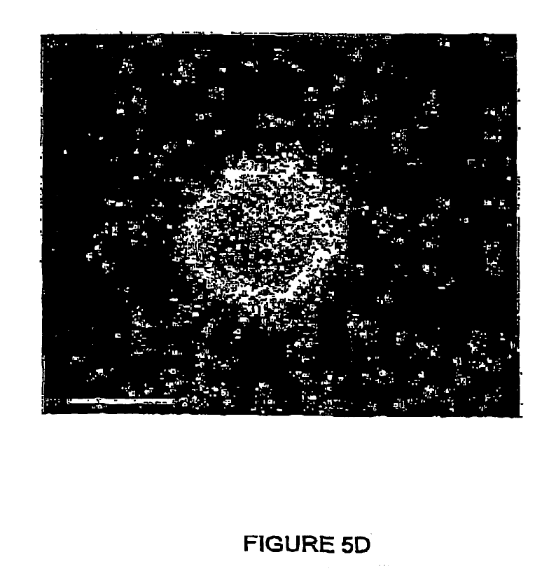

In initial studies we found little evidence for the transport of DNP-GS by cMOAT expressed in the mouse leukemia cell line L1210 (data not shown). This lack of function suggests that the protein did not localize to the cell membrane in these cells. To confirm this observation, cMOAT-gfp was expressed in L1210 cells and was found to localize predominantly in intracellular vesicles with only minor membrane localization (FIG. 5A). Since ΔcMOAT-gfp localized basolaterally in MDCK cells, we were interested to determine whether it was able to localize in the L1210 cell membrane. In contrast to cMOAT-gfp, ΔcMOAT-gfp expressed in L1210 cells almost exclusively localizes to the cell membrane (FIG. 5B). To confirm the localization, we studied cells stably expressing cMOAT and ΔcMOAT without the GFP tag by immunofluorescence. cMOAT was detected intracellularly and had a vesicular localization within the cell (FIG. 5C), the same distribution as shown in FIG. 5A. The ΔcMOAT polypeptide was detected in the cell membrane (FIG. 5D), exhibiting the same localization as ΔcMOAT-gfp shown in FIG. 5B.

These data suggest that the deletion of the TKF motif from cMOAT allows the successful targeting of the protein to the membrane of non-polarized L1210 cells. However, some L1210 cells expressing ΔcMOAT or ΔcMOAT-gfp demonstrated a degree of intracellular accumulation.

2,4-Dinitrophenyl Glutathione Transport

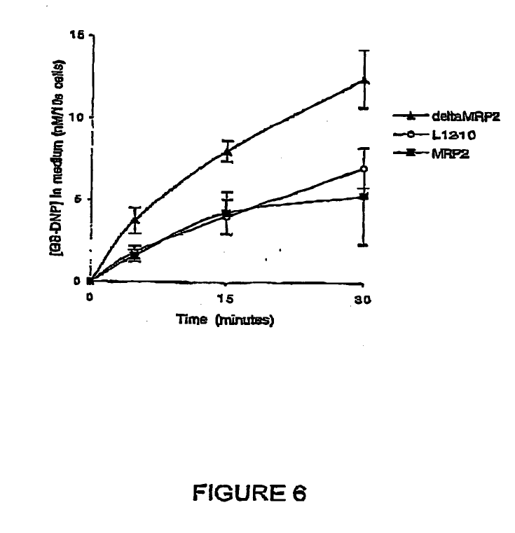

L1210 cells are non-adherent and non-polarized, and can be potentially used as a convenient cell line for assessing the transport function of cMOAT. As shown in FIG. 6, L1210 cells stably expressing ΔcMOAT showed a significantly higher efflux of DNP-GS compared to control L1210 cells or L1210 cells expressing native cMOAT protein.

Discussion

Human cMOAT specifically localizes to the apical membrane of polarized epithelial cells in the liver and kidney. This localization can be replicated experimentally in MDCK cells (Evers et al., (1998) J. Clin. Invest. 101, 1310-1319; and Cui et a/, (1999) Mol. Pharmacol. 55, 929-937) and LLC-PK1 cells (Chen et al., (1999) Mol. Phar-macol. 56, 1219-1228; and Kawabe et al., (1999) FEBS Lett. 456, 327-331), and we demonstrate in this study that an cMOAT-gfp fusion protein also localizes to the apical membrane (FIG. 1). This allowed us to undertake mutational analysis to determine targeting signals for apical localization. Deletion of the three amino acids from the C terminus of cMOAT (ΔcMOAT-gfp) caused a dramatic change in the targeting of the protein to the basolateral membrane, and to a lesser extent in the lateral and apical membranes, in MDCK cells. The mutant's localization in a polarized cell was similar to that of MRP1 (FIG. 2), which does not normally have a T-K-F motif. This observation indicates that the three-amino acid motif targets cMOAT to the apical membrane and dominates any basolateral targeting signals.

Moreover, deletion of the T-K-F motif also produces a modified cMOAT polypeptide that is localized in the plasma cell membrane of non polarized L1210 cells. In contrast, wild type cMOAT is predominantly intracellular in L1210 cells.

To further characterize the T-K-F motif, alanine was introduced into the position of each residue separately, and an additional mutant was made in which all three residues were replaced by alanine.

Our data suggest that a functional T-K-F motif is characterized by the consensus sequence S/T-X-Hy, wherein X represents any amino acid and Hy is a hydrophobic residue (Songyang et al., (1997) Science 275, 73-77). For example, the T1543A mutant did exhibit modified targeting compared with the native cMOAT protein, allowing both basolateral and apical targeting, (i.e. non-polarized targeting), and also an increased accumulation in vesicles, suggesting some instability in the targeting mechanism. This conclusion is also consistent with the results obtained by the TKF-AAA mutant. The F1545A mutant did not alter normal targeting, suggesting that alanine at position 1545 is sufficiently hydrophobic for normal targeting to occur. Accordingly, any residue (X) may be tolerated at position 1545 of cMOAT, but not at position 1544, since K1544A was also targeted to the basolateral membrane.

Interestingly, the serine residue at position 1542 forms a predicted phosphorylation site, and mutation of this serine residue caused the fusion protein to localize in sub-apical vesicles. These data suggest that S1542 may be phosphorylated and could regulate recruitment into the apical membrane.

There is a notable difference in the sorting of cMOAT in L1210 cells compared with the MDCK cells. When expressed in L1210 cells, cMOAT transport function was minimal (results not shown). Immunofluorescence studies of cells expressing cMOAT and confocal imaging of cells expressing cMOAT-gfp both confirm the intracellular localization of the protein in L1210 cells (FIG. 5). Deletion of the T-K-F motif from cMOAT and cMOAT-gfp allows the mutant protein to be expressed in the plasma membrane, suggesting that it is this apical targeting motif that excludes the native cMOAT from the membrane. The stability of the protein in the membrane also differs between MDCK cells and L1210 cells. In MDCK cells ΔcMOAT-gfp has basolateral localization in the majority of cells.

### EXAMPLE 2

### TKF Motifs in Other ABC Transporter Polypeptides Derived from Sequence Alignments and Molecular Modeling

Experimental Procedure

The protein sequence of the C-terminal cytoplasmic domains of 37 ABC transporters from the P-glycoprotein and MRP subfamilies were aligned with the histidine permease (HisP) sequence using the ClustalW alignment program. The multiple sequence alignment was used with the coordinates of the HisP crystal structure (Hung et al., (1998) Nature 396, 703-707) to generate a homology model of the C-terminal cytoplasmic domain from MRP1 and cMOAT using BioNavigator at the ANGIS Internet site (BioNavigator by eBioinformatics Pty. Ltd.). The models were generated using the Rigorous Models software (Abagyan et al., (1994) J. Comp. Chem. 15,488-506) and presented using Swiss Pdb Viewer (v3.6b3) (Guex et al., (1997) Electrophoresis 18, 2714-2723).

Results

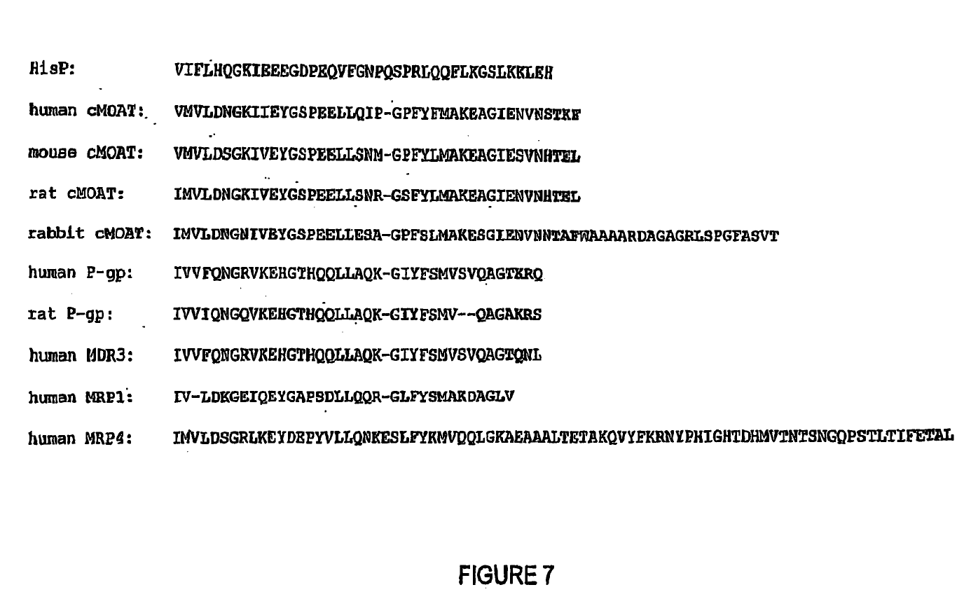

The alignment represented in FIG. 7 shows that those MRP proteins that localize to the apical membrane (cMOAT from four species) have a C-terminal T-K-F motif when compared with MRP1, MRP3, MRP5, and MRP6, which are targeted to the basolateral membrane. The P-gp, MDR3, and MRP4 proteins also have a potential T-K-F motif at their C termini.

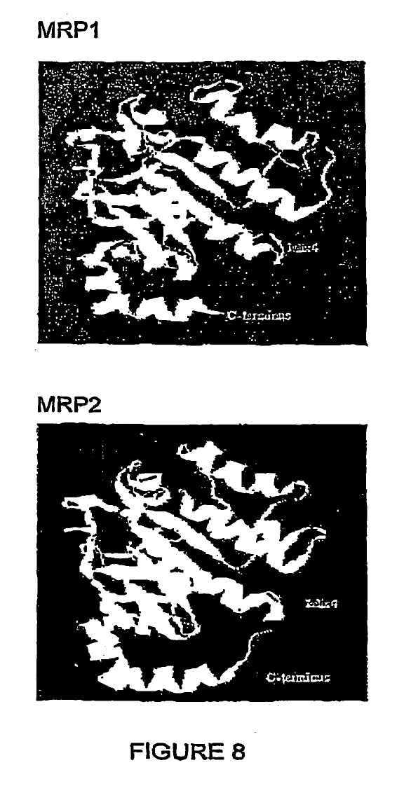

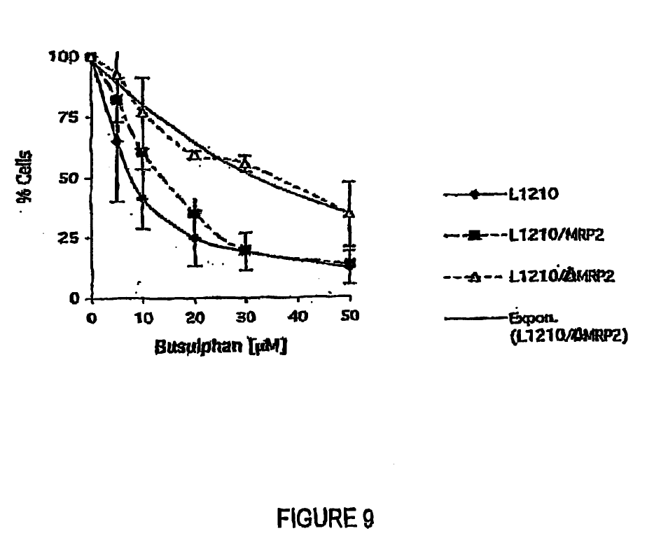

The structural coordinates for the ATP binding subunit of histidine permease from Salmonella typhimurium (Hung et al., (1998) Nature 396, 703-707) and the full ABC transporter sequence alignment allowed the construction of homology models of the equivalent regions of MRP1 and cMOAT (FIG. 8). Since the C-terminal motif of cMOAT extends beyond the alignment with HisP, the exact position of the T-K-F motif residues cannot be predicted. However, the models predict that the T-K-F motif is positioned on the outside of the protein, away from the ATP binding cassette and regions involved in the cytoplasmic subunit interface. In addition, the external position of the motif would favor interactions with other proteins involved in the targeting process.

Discussion

The deletion of the T-K-F motif increases the sequence similarity of cMOAT to MRP1 and results in the same basolateral targeting as observed for MRP1. To investigate the tertiary structure of the subunit and the position of the motif, homology models of both MRP1 and cMOAT were created based the crystal structure of HisP. Comparisons of the homology models clearly show the difference in length of the C terminus of MRP1 and cMOAT. It is not clear whether the TKF motif is solely responsible for the apical localization or whether it is the spatial arrangement of the extension and the predicted T-K-F motif that allows binding/modification to another part of the ABC transporter protein. From the homology model of cMOAT it appears likely that the T-K-F motif is available for interaction and not buried within the subunit Also, the position of the C-terminus in this model suggests that the motif does not interact with functionally significant areas such as the ATP binding sites. This is further supported by the ability of the deletion mutant ΔcMOAT to transport 2,4-dinitrophenyl glutathione when expressed in L1210 cells (FIG. 6).

The attachment of GFP to the C-terminus of MRP1 or cMOAT did not interfere with correct targeting of cMOAT. Based on the homology models in FIG. 8, the position of the C-terminal helix indicates that GFP would sit on the outside of the subunit. The position of GFP therefore would suggest that the T-K-F motif does not need to be freely exposed and carboxylated to function. In support of this, rabbit cMOAT has a predicted T-K-F motif in the same position as human, mouse, and rat cMOAT but also comprises a further 21 amino acids downstream (FIG. 7).

The GFP fusion proteins were expressed at consistent levels under the CMV promoter of the EGFP-N1 vector. The cMOAT-gfp fusion protein localized apically in the majority of polarized MDCK cells as represented in FIG. 1. cMOAT has been found to be expressed in ovarian cancer cells lines (Kool et al., (1997) Cancer Res. 57, 3537-3547), renal clear cell carcinomas (Schaub et al., (1999) J. Am. Soc. Nephrol. 10,1159-1169), lung, gastric, and colorectal cancer cells (Narasaki et al., (1997) Biochem. Biophys. Res. Comm. 240, 606-611).

Alignment of cMOAT with the basolateral MRP proteins MRP1, MRP3, MRP5, and MRP6 shows the absence of the motif in the basolateral transporters (FIG. 7). Based on this alignment, the cMOAT residues 1539-1545 may play a role in the targeting mechanism as this is the full length of the extension of the C terminus compared with the basolateral proteins.

### EXAMPLE 3

Modified cMOAT Polypeptides Confer Resistance to Busulfan on L1210 Cells

Busulfan is normally conjugated to glutathione in the cytoplasm of cells by glutathione-S-transferase (Czerwinski et al. (1996) Drug Met. Dispos. 24, 1015-1019), indicating that the conjugated product is possibly a substrate for cMOAT. Accordingly, the ability of modified cMOAT polypeptides to confer resistance to Busulfan was determined in L1210 cells. In particular, the ΔcMOAT polypeptide having the amino acid sequence set forth in SEQ ID NO: 4, was expressed in L1210 cells as described in Example 1. The transfected cells were exposed to a range of concentrations of Busulfan. The survival of wild type L1210 cells, and transfected L1210 cells expressing either native cMOAT or ΔcMOAT, was determined in the presence of Busulfan. Survival was also assessed relative to the growth of cells that had not been exposed to Busulfan. Cell growth and survival were assayed using standard procedures (Denizot et al (1986), J. Immunol. Methods 89, 271-277). Data presented in FIG. 8 indicate that those cells expressing ΔcMOAT had significantly enhanced resistance to Busulfan than non-transfected L1210 cells, or L1210 cells expressing native cMOAT (i.e. a 2-fold increase in the IC₅₀ was determined for cells expressing ΔcMOAT).

Based upon the functional equivalence of the ΔcMOAT polypeptide to the other modified cMOAT polypeptides set forth in SEQ ID NOs: 6, 10, 12, and 16, those skilled in the art will be aware from this disclosure of the utility of those other sequences in conferring resistance to any chemical on a non-polarized cell.

### EXAMPLE 4

### Use of modified ABC Transporter Polypeptides to Screen for Modulators of ABC Transporters

By targeting a modified cMOAT polypeptide to the cell membrane of a suspension cell of the haematopoietic lineage, such as, for example, L1210 cells or Jurkat cells, therapeutic agents that are transported by cMOAT, or novel therapeutic agents that modulate cMOAT, are detected by virtue of their ability to be transported from the cell. Cells that are stably transfected with a mutated cMOAT cDNA sequence encoding a modified cMOAT polypeptide are incubated with such novel therapeutic agents at levels that are not cytotoxic. Following incubation, the supernatants of cells are analyzed by HPLC to determine whether or not the agents are metabolized. Alternatively in the case of fluorescent chemical agents, the cells are examined by flow cytometry, for a decrease in fluorescence due to cMOAT export function. Using a known fluorescent substrate for cMOAT, such as Fluo-3, potential modulators of cMOAT are tested by detecting inhibition of the transport of the fluorescent compound, measured by flow cytometry.

Preferably, L1210 cells expressing modified ABC transporter polypeptides (e.g. any one of the modified cMOAT polypeptides set forth in SEQ ID NOs: 4, 6, 10, 12, or 16; the modified MDR3 polypeptide of SEQ ID NO: 49, or the modified MRP4 polypeptide of SEQ ID NO: 51) are incubated with a suitable substrate, such as, for example, 1-chloro-2,4-dinitrobenzene or mono-chlorobimane (thiolyte, Calbiochem) or 7-chloro-4-nitrobenz-2-oxa-1,3-diazole (Sigma), which are assayed by measuring absorbance or fluorescence. The transfected cells are then separately incubated with: (i) a candidate inhibitor or candidate activator of the corresponding native ABC transporter polypeptide, being native cMOAT, MDR3, or MRP4, as appropriate (i.e. the test sample); and (ii) no added candidate compound (i.e. the control sample). The rate of efflux of the glutathione conjugate from the cells is determined for both the test sample and the control sample, by measuring the absorbance or fluorescence of the glutathione conjugate in the medium. Those samples wherein the absorbance or fluorescence of the test sample is significantly different from the absorbance or fluorescence of the control sample are selected. Candidate compounds that induce higher efflux of glutathione conjugate from the cell (e.g. higher absorbance or fluorescence of the test sample relative to the absorbance or fluorescence of the control sample) are classified as agonists of the native ABC transporter polypeptide, whilst candidate compounds that reduce efflux of glutathione conjugate from the cell (i.e. reduced absorbance or fluorescence of the test sample relative to the absorbance or fluorescence of the control sample) are classified as antagonists of the native ABC transporter polypeptide. Optionally, this screen is readily adapted to a high throughput format, such as, for example, by FACS screening of multiple samples, by virtue of the capability of detecting the glutathione conjugate.

### EXAMPLE 5

### A Modified MDR3 Polypeptide

Introduction

We sought to establish the localization of a modified MDR3 polypeptide lacking the putative TKF motif (i.e. SEQ ID NO: 49) in both L1210 cells and MDCK cells, using the methods established for cMOAT as described in Example 1. Determination of the localization of modified MDR3 is facilitated by expressing the polypeptide as a green fluorescent protein (GFP) fusion protein.

Experimental Procedures

A gene construct that encodes modified MDR3 as a fusion protein with GFP GFP is fused to the C-terminal region of the human MDR3 polypeptide, to facilitate detection of the localization of the MDR3-gfp fusion protein, as described below.

Human MDR3 cDNA is amplified from the native MDR3-encoding cDNA (Accession No. XM 029057) by polymerase chain reaction using PfuTurbo DNA polymerase (Stratagene), to remove the stop codon and introduce restriction enzyme sites suitable for cloning. DNA encoding modified MDR3 is amplified using a sense primer that adds an NheI site immediately adjacent to the start codon, as follows:

(SEQ ID NO: 59)

5′-AGCGCTAGCGATGGATCTTGAGGCGGCAAAG-3′;

and an antisense primer that adds an AgeI site after the final codon and removes the stop codon, as follows:

| | |
|---|---|
| 5′-TACGGTACCGGTGCCCCAGCCTGGACA-3′; | (SEQ ID NO: 60) |

The polymerase chain reaction product is digested with NheI/AgeI and ligated into the NheI/AgeI-digested EGFP-N1 vector (CLONTECH), to introduce the modified MDR3-encoding nucleotide sequence immediately upstream and in-frame with the GFP-encoding nucleotide sequence in that vector.

The modified MDR3-gfp construct (1 μg of DNA per transfection) is transfected into MDCK cells and L1210 cells using a LipofectAMINE transfection kit (Life Technologies, Inc.). Transfections of MDCK cells are carried out using Transwell plates (Costar, 24 mm×3 μm polycarbonate membrane) to enable cell polarization. Cells are imaged using a NikonTE300 inverted microscope linked to a Radiance 2000. Laser Scanning System for confocal microscopy and Lasersharp 2000 imaging software (Bio-Rad).

Expression of the Modified MDR3 Polypeptide Without a gfp Tag

The nucleotide sequence encoding the modified MDR3 polypeptide (i.e. SEQ ID NO: 48) is prepared using the QuikChange site-directed mutagenesis kit to facilitate cloning without nucleotide sequences encoding a GFP tag. To produce this construct, template DNA comprising the wild-type MDR3 cDNA in the mammalian expression vector pRc/CMV (Invitrogen) (Taniguchi et al., (1996) Cancer Res. 56, 4124-4129) is amplified using primers that do not include nucleotides encoding the T-K-F motif of native MDR3. Successful mutagenesis of clones is confirmed by sequencing, and those clones, in the pRc/CMV vector, are transfected into L1201 cells.

The transport of [³H]paclitaxel is determined from L1210 cells expressing the modified MDR3 polypeptide and compared to the efflux of [³H]paclitaxel from control L1210 cells not ectopically expressing any MDR3 polypeptide.

Results

Localization of the Modified MDR-gfp Fusion Protein

To conveniently detect the localization of the proteins under investigation, GFP fusion proteins are produced and their localization is visualized using confocal microscopy to visualize the fluorescent product, as described supra.

When expressed in polarized MDCK cells, the modified MDR3-gfp polypeptide is found to have a modified localization compared to native MDR3, wherein the modified polypeptide localizes is no longer predominantly in the apical membrane. cells.

In L1210 cells, the modified MDR3 polypeptide is found in the plasma membrane

[³H]paclitaxel Transport

L1210 cells stably expressing the modified MDR3 polypeptide without a GFP tag have a significantly higher efflux of [³H]paclitaxel compared to control L1210 cells.

### EXAMPLE 6

### A Modified MRP4 Polypeptide

Introduction

We also sought to establish the localization of a modified MRP4 polypeptide lacking the putative TKF motif (i.e. SEQ ID NO: 51), in both L1210 cells and MDCK cells, using the methods described in Examples 1 and 5, by analyzing the localization of a modified MRP4-gfp fusion polypeptide ectopically expressed in these cell lines.

Experimental Procedures

A gene construct that encodes modified MDR3 as a fusion protein with GFP GFP is fused to the C-terminal region of the human MRP4 polypeptide, to facilitate detection of the localization of the MRP4-gfp fusion protein, as described below.

Human MRP4 cDNA is amplified from the native MRP4-encoding cDNA (Accession No. XM 036453) by polymerase chain reaction using PfuTurbo DNA polymerase (Stratagene), to remove the stop codon and introduce restriction enzyme sites suitable for cloning. The cDNA encoding modified MRP4 is amplified using a sense primer that adds an NheI site immediately adjacent to the start codon, as follows:

(SEQ ID NO: 61)

5′-AGCGCTAGCGATGCTGCCCGTGTACCAGGAG-3′;

and an antisense primer that adds an AgeI site after the final codon and removes the stop codon, as follows:

| | |
|---|---|
| 5′-TACGGTACCGGTGCCTCGAAAATAGTT-3′; | (SEQ ID NO: 62) |

The polymerase chain reaction product is digested with NheI/AgeI and ligated into the NheI/AgeI-digested EGFP-N1 vector (CLONTECH), to introduce the modified MRP4 encoding nucleotide sequence immediately upstream and in-frame with the GFP-encoding nucleotide sequence in that vector.

The modified MRP4-gfp construct (1 μl of DNA per transfection) is transfected into MDCK cells and L1210 cells using a LipofectAMINE transfection kit (Life Technologies, Inc.). Transfections of MDCK cells are carried out using Transwell plates (Costar, 24 mm×3 μm polycarbonate membrane) to enable cell polarization. Cells are imaged using a NikonTE300 inverted microscope linked to a Radiance 2000 Laser Scanning System for confocal microscopy and Lasersharp 2000 imaging software (Bio-Rad).

Expression of the Modified MRP4 Polypeptide without a gfp Tag

The nucleotide sequence encoding the modified MRP4 polypeptide (i.e. SEQ ID NO: 50) is prepared using the QuikChange site-directed mutagenesis kit to facilitate cloning without nucleotide sequences encoding a GFP tag. To produce this construct, template DNA comprising the wild-type MRP4 cDNA cloned into the mammalian expression vector pRc/CMV (Invitrogen) (Taniguchi et al., (1996) Cancer Res. 56, 4124-4129), is amplified using primers that do not include nucleotides encoding the T-K-F motif of native MRP4. Successful mutagenesis of clones is confirmed by sequencing, and those clones, in the pRc/CMV vector, are transfected into L1201 cells.

Radiolabeled 6-mercaptopurine is added to L1210 cells expressing the modified MRP4 polypeptide and the efflux of 6-thio-IMP compared to the efflux of 6-thio-IMP from L1210 cells expressing native MRP4, or alternatively, the efflux of 6-thio-IMP from control L1210 cells not ectopically expressing any MRP4 polypeptide.

Results

Localization of the Modified MRP4-gfp Fusion Protein

To conveniently detect the localization of the proteins under investigation, GFP fusion proteins are produced and their localization is visualized using confocal microscopy to visualize the fluorescent product, as described supra.

When expressed in polarized MDCK cells, the modified MRP4-gfp polypeptide is found to have a modified localization compared to native MRP4, wherein the modified polypeptide localizes is no longer predominantly in the apical membrane. cells.

In L1210 cells, the modified MRP4 polypeptide is found in the plasma membrane.

6-mercaptopurine Transport

L1210 cells stably expressing the modified MRP4 polypeptide without a GFP tag have a significantly higher efflux of 6-thio-IMP compared to control L1210 cells or L1210 cells expressing native MRP4 protein.

We claim:

1. A modified ABC transporter polypeptide that is localized predominantly in the basolateral membrane of a polarized cell or in the plasma membrane of a non-polarized cell, said modified ABC transporter polypeptide consisting of the amino acid sequence of a native ABC transporter polypeptide that normally accumulates in the apical (canalicular) membrane of a polarized cell wherein one or more amino acid residues of a C-terminal tripeptide T-K-F motif of said native ABC transporter polypeptide having a sequence set forth in any one of SEQ ID NOs: 52 to 58 is substituted or deleted.

2. The modified ABC transporter polypeptide of claim 1, wherein the threonine at the first position of the tripeptide T-K-F motif is substituted with a different amino acid residue.

3. The modified ABC transporter of claim 2 wherein the different amino acid residue is alanine.

4. The modified ABC transporter polypeptide of claim 1, wherein the lysine at the second position of the tripeptide T-K-F motif is substituted with a different amino acid residue.

5. The modified ABC transporter polypeptide of claim 4, wherein the different amino acid residue is alanine or proline.

6. The modified ABC transporter polypeptide of claim 1, wherein three amino acid residues of the tripeptide T-K-F motif are substituted with different amino acid residues.

7. The modified ABC transporter polypeptide of claim 6, wherein each of the three amino acid residues of the tripeptide T-K-F motif is substituted with alanine.

8. The modified ABC transporter polypeptide of claim 6, wherein the three amino acid residues of the tripeptide T-K-F motif are substituted respectively with alanine, proline and valine.

9. The modified ABC transporter polypeptide of claim 1, wherein three amino acid residues of the tripeptide T-K-F motif are deleted.

10. The modified ABC transporter polypeptide of claim 1 wherein the native ABC transporter polypeptide that normally accumulates in the apical (canalicular) membrane of a polarized cell is selected from the group consisting of canalicular multispecific organic anion transporter (cMOAT), MDR3 and MRP4.

11. The modified ABC transporter polypeptide of claim 10, wherein the native ABC transporter polypeptide is human cMOAT, human MDR3, or human MRP4.

12. A fusion polypeptide comprising the modified ABC transporter of claim 1 covalently linked to green fluorescent protein (gfp).

13. A modified canalicular multispecific organic anion transporter (cMOAT) polypeptide localized predominantly in the basolateral membrane of a polarized cell or in the plasma membrane of a non-polarized cell, said modified cMOAT polypeptide consisting of the amino acid sequence of a native cMOAT polypeptide wherein one or more amino acid residues of a C-terminal tripeptide T-K-F motif of said native cMOAT polypeptide having a sequence set forth in SEQ ID NO: 52 or SEQ ID NO: 53 or SEQ ID NO: 55 is substituted or deleted.

14. The modified cMOAT polypeptide of claim 13 wherein one or more amino acid residues of the C-terminal tripeptide T-K-F motif of said native cMOAT polypeptide having the sequence set forth in SEQ ID NO: 52 is substituted or deleted.

15. The modified cMOAT polypeptide of claim 14, wherein the threonine at the first position of the tripeptide T-K-F motif is substituted with a different amino acid residue.

16. The modified cMOAT of claim 15 wherein the different amino acid residue is alanine.

17. The modified cMOAT polypeptide of claim 14 wherein the lysine at the second position of the tripeptide T-K-F motif is substituted with a different amino acid residue.

18. The modified cMOAT polypeptide of claim 17 wherein the different amino acid residue is alanine or proline.

19. The modified cMOAT polypeptide of claim 14, wherein three amino acid residues of the tripeptide T-K-F motif are substituted with different amino acid residues.

20. The modified cMOAT polypeptide of claim 19, wherein each of the three amino acid residues of the tripeptide T-K-F motif is substituted with alanine.

21. The modified cMOAT polypeptide of claim 19, wherein the three amino acid residues of the tripeptide T-K-F motif are substituted respectively with alanine, proline and valine.

22. The modified cMOAT polypeptide of claim 13, wherein three amino acid residues of the tripeptide T-K-F motif are deleted.

23. A fusion polypeptide comprising the modified cMOAT of claim 13 covalently linked to green fluorescent protein (gfp).

24. A modified MDR3 polypeptide localized predominantly in the basolateral membrane of a polarized cell or in the plasma membrane of a non-polarized cell, said modified MDR3 polypeptide consisting of the amino acid sequence of a native MDR3 polypeptide wherein the C-terminal tripeptide T-K-F motif of said native MDR3 polypeptide having the sequence set forth in SEQ ID NO: 57 is deleted.

25. A fusion polypeptide comprising the modified MDR3 of claim 24 covalently linked to green fluorescent protein (gfp).

26. A modified MRP4 polypeptide localized predominantly in the basolateral membrane of a polarized cell or in the plasma membrane of a non-polarized cell, said modified MRP4 polypeptide consisting of the amino acid sequence of a native MRP4 polypeptide wherein the C-terminal tripeptide T-K-F motif of said native MRP4 polypeptide having the sequence set forth in SEQ ID NO: 54 is deleted.

27. A fusion polypeptide comprising the modified MRP4 of claim 26 covalently linked to green fluorescent protein (gfp).

28. A modified canalicular multispecific organic anion transporter (cMOAT) polypeptide that consists of the amino acid sequence set forth in SEQ ID NO: 4.

29. A fusion polypeptide comprising the modified cMOAT polypeptide of claim 28 covalently linked to green fluorescent protein (gfp).

30. A modified canalicular multispecific organic anion transporter (cMOAT) polypeptide that comprises the amino acid sequence set forth in SEQ ID NO: 6.

31. A fusion polypeptide comprising the modified cMOAT polypeptide of claim 30 covalently linked to green fluorescent protein (gfp).

32. A modified canalicular multispecific organic anion transporter (cMOAT) polypeptide that comprises the amino acid sequence set forth in SEQ ID NO: 10.

33. A fusion polypeptide comprising the modified cMOAT polypeptide of claim 32 covalently linked to green fluorescent protein (gfp).

34. A modified canalicular multispecific organic anion transporter (cMOAT) polypeptide that comprises the amino acid sequence set forth in SEQ ID NO: 12.

35. A fusion polypeptide comprising the modified cMOAT polypeptide of claim 34 covalently linked to green fluorescent protein (gfp).

36. A modified canalicular multispecific organic anion transporter (cMOAT) polypeptide that comprises the amino acid sequence set forth in SEQ ID NO: 16.

37. A fusion polypeptide comprising the modified cMOAT polypeptide of claim 36 covalently linked to green fluorescent protein (gfp).

38. A modified MDR3 polypeptide that consists of the amino acid sequence set forth in SEQ ID NO: 49.

39. A fusion polypeptide comprising the modified MDR3 polypeptide of claim 38 covalently linked to green fluorescent protein (gfp).

40. A modified MRP4 polypeptide that consists of the amino acid sequence set forth in SEQ ID NO: 51.

41. A fusion polypeptide comprising the modified MRP4 polypeptide of claim 40 covalently linked to green fluorescent protein (gfp).

42. An isolated nucleic acid that comprises a nucleotide sequence encoding a modified ABC transporter polypeptide that consists of the amino acid sequence of a native ABC transporter polypeptide that normally accumulates in the apical (canalicular) membrane of a polarized cell wherein one or more amino acid residues of a C-terminal tripeptide T-K-F motif of said native ABC transporter polypeptide having an amino acid sequence set forth in any one of SEQ ID NOs: 52 to 58 is substituted or deleted.

43. The isolated nucleic acid of claim 42 wherein said nucleotide sequence includes a mutation relative to the corresponding wild-type gene selected from the group consisting of:
(i) a deletion of nucleotides from the 3′ end of the coding region of said wild-type gene sufficient to encode a modified ABC transporter polypeptide lacking said T-K-F motif;
(ii) a substitution of nucleotides within the 3-end of the coding region of said wild-type gene sufficient to encode a modified ABC transporter wherein the first amino acid residue of said T-K-F motif is substituted;
(iii) a substitution of nucleotides within the 3′-end of the coding region of said wild-type gene sufficient to encode a modified ABC transporter wherein the second amino acid residue of said T-K-F motif is substituted; and
(iv) a substitution of nucleotides within the 3′-end of the coding region of said wild-type gene sufficient to encode a modified ABC transporter wherein all three amino acid residues of said T-K-F motif are substituted.

44. The isolated nucleic acid of claim 43 wherein the modified ABC transporter comprises the substitution of threonine for alanine at the first amino acid residue of said T-K-F motif.

45. The isolated nucleic acid of claim 43 wherein the modified ABC transporter comprises the substitution of lysine for alanine or proline at the second amino acid residue of said T-K-F motif.

46. The isolated nucleic acid of claim 43 wherein the modified ABC transporter comprises the substitution of each of the three amino acid residues of the tripeptide T-K-F motif for alanine.

47. The isolated nucleic acid of claim 43 wherein the modified ABC transporter comprises the substitution of the three amino acid residues of the tripeptide T-K-F motif respectively for alanine, proline and valine.

48. The isolated nucleic acid of claim 43 encoding a modified canalicular multispecific organic anion transporter (cMOAT), modified MDR3 or modified MRP4 polypeptide.

49. The isolated nucleic acid of claim 48 wherein the modified cMOAT, modified MDR3 or modified MRP4 polypeptide is modified human cMOAT, modified human MDR3, or modified human MRP4.

50. An isolated nucleic acid encoding a modified canalicular multispecific organic anion transporter (cMOAT) polypeptide, said nucleic acid consisting of a nucleotide sequence selected from the group consisting of:
(i) the nucleotide sequence set forth in SEQ ID NO: 3; and
(ii) a nucleotide sequence encoding the amino acid sequence set forth in SEQ ID NO: 4.

51. An isolated nucleic acid encoding a modified canalicular multispecific organic anion transporter (cMOAT) polypeptide, said nucleic acid comprising a nucleotide sequence selected from the group consisting of
(i) the nucleotide sequence set forth in SEQ ID NO: 5; and
(ii) a nucleotide sequence encoding the amino acid sequence set forth in SEQ ID NO: 6.

52. An isolated nucleic acid encoding a modified canalicular multispecific organic anion transporter (cMOAT) polypeptide, said nucleic acid comprising a nucleotide sequence selected from the group consisting of:
(i) the nucleotide sequence set forth in SEQ ID NO: 9; and
(ii) a nucleotide sequence encoding the amino acid sequence set forth in SEQ ID NO: 10.

53. An isolated nucleic acid encoding a modified canalicular multispecific organic anion transporter (cMOAT) polypeptide, said nucleic acid comprising a nucleotide sequence selected from the group consisting of:
(i) the nucleotide sequence set forth in SEQ ID NO: 11; and
(iii) a nucleotide sequence encoding the amino acid sequence set forth in SEQ ID NO: 12.

54. An isolated nucleic acid encoding a modified canalicular multispecific organic anion transporter (cMOAT) polypeptide, said nucleic acid comprising a nucleotide sequence selected from the group consisting of:
(i) the nucleotide sequence set forth in SEQ ID NO: 15; and
(ii) a nucleotide sequence encoding the amino acid sequence set forth in SEQ ID NO: 16.

55. An isolated nucleic acid encoding a modified MDR3 polypeptide, said nucleic acid consisting of a nucleotide sequence selected from the group consisting of:
(i) the nucleotide sequence set forth in SEQ ID NO: 48; and
(ii) a nucleotide sequence encoding the amino acid sequence set forth in SEQ ID NO: 49.

56. An isolated nucleic acid encoding a modified MRP4 polypeptide, said nucleic acid consisting of a nucleotide sequence selected from the group consisting of:
(i) the nucleotide sequence set forth in SEQ ID NO: 50; and
(ii) a nucleotide sequence encoding the amino acid sequence set forth in SEQ ID NO: 51.

57. A method of producing nucleic acid encoding a modified ABC transporter polypeptide comprising deleting or substituting a portion of the coding region of nucleic acid encoding an ABC transporter polypeptide that accumulates in the apical (canalicular) membrane of a polarized cell, said portion encoding the first or second amino acid residue of the C-terminal tripeptide T-K-F motif of said ABC transporter or all three amino acid residues of said C-terminal tripeptide T-K-F motif, wherein said motif has a sequence set forth in any one of SEQ ID NOs: 52 to 58.

58. A method of producing nucleic acid encoding a modified human canalicular multispecific organic anion transporter (cMOAT) polypeptide comprising deleting or substituting a portion of the coding region set forth in SEQ ID NO: 1, said portion encoding the first or second amino acid residue of the C-terminal tripeptide T-K-F motif of said cMOAT or all three amino acid residues of said C-terminal tripeptide T-K-F motif, wherein said motif has a sequence set forth in SEQ ID NO: 52.

59. The method of claim 58 wherein deleting or substituting a portion of the coding region comprises amplifying nucleic acid encoding cMOAT using a primer comprising a deletion or substitution within the sequence corresponding or complementary to the portion of the coding region encoding the first or second amino acid residue of the C-terminal tripeptide T-K-F motif of said cMOAT or all three amino acid residues of said C-terminal tripeptide T-K-F motif; and selecting the amplified nucleic acid wherein the deletion or substitution is introduced into the remainder of the coding region of SEQ ID NO: 1.

60. The method of claim 58 wherein the primer comprises or is complementary to a nucleotide sequence selected from the group consisting of SEQ ID NO: 26; SEQ ID NO: 27; SEQ ID NO: 29; SEQ ID NO: 30; SEQ ID NO: 32; and SEQ ID NO: 33.

61. A method of producing nucleic acid encoding a modified human MDR3 polypeptide comprising deleting a portion of the coding region encoding a native human MDR3 polypeptide, said portion encoding the C-terminal tripeptide T-K-F motif of said native human MDR3, wherein said motif has a sequence set forth in SEQ ID NO: 57.

62. The method of claim 61 wherein deleting a portion of the coding region comprises amplifying nucleic acid encoding native human MDR3 using a primer that hybridizes to the 3′-end of said coding region or its complement wherein said primer comprises a deletion within the sequence corresponding or complementary to the portion of said coding region encoding said C-terminal tripeptide T-K-F motif; and selecting the amplified nucleic acid wherein the deletion is introduced into the remainder of the coding region encoding MDR3.

63. The method of claim 62 wherein the primer comprises or is complementary to the nucleotide sequence set forth in SEQ ID NO: 60.

64. A method of producing nucleic acid encoding a modified human MRP4 polypeptide comprising deleting a portion of the coding region encoding a native human MRP4 polypeptide, said portion encoding the C-terminal tripeptide T-K-F motif of said native human MRP4, wherein said motif has a sequence set forth in SEQ ID NO: 54.

65. The method of claim 64 wherein deleting a portion of the coding region comprises amplifying nucleic acid encoding native human MRP4 using a primer that hybridizes to the 3′-end of said coding region or its complement wherein said primer comprises a deletion within the sequence corresponding or complementary to the portion of said coding region encoding said C-terminal tripeptide T-K-F motif; and selecting the amplified nucleic acid wherein the deletion is introduced into the remainder of the coding region encoding MRP4.

66. The method of claim 65 wherein the primer comprises or is complementary to the nucleotide sequence set forth in SEQ ID NO: 62.

67. A gene construct comprising the isolated nucleic acid of claim 42 encoding a modified ABC transporter polypeptide wherein said nucleic acid is in operable connection with a promoter sequence to facilitate expression of said polypeptide in a cell.

68. The gene construct of claim 67 wherein the nucleic acid is in the same reading frame as nucleic acid encoding green fluorescent protein (gfp) such that the modified ABC transporter polypeptide is capable of being expressed as a fusion polypeptide with said gfp.

69. A gene construct comprising the isolated nucleic acid of claim 50 encoding a modified ABC transporter polypeptide wherein said nucleic acid is in operable connection with a promoter sequence to facilitate expression of said polypeptide in a cell.

70. The gene construct of claim 69 wherein the nucleic acid is in the same reading frame as nucleic acid encoding green fluorescent protein (gfp) such that the modified ABC transporter polypeptide is capable of being expressed as a fusion polypeptide with said gfp.

71. A gene construct comprising the isolated nucleic acid of claim 51 encoding a modified ABC transporter polypeptide wherein said nucleic acid is in operable connection with a promoter sequence to facilitate expression of said polypeptide in a cell.

72. The gene construct of claim 71 wherein the nucleic acid is in the same reading frame as nucleic acid encoding green fluorescent protein (gfp) such that the modified ABC transporter polypeptide is capable of being expressed as a fusion polypeptide with said gfp.

73. A gene construct comprising the isolated nucleic acid of claim 52 encoding a modified ABC transporter polypeptide wherein said nucleic acid is in operable connection with a promoter sequence to facilitate expression of said polypeptide in a cell.

74. The gene construct of claim 73 wherein the nucleic acid is in the same reading frame as nucleic acid encoding green fluorescent protein (gfp) such that the modified ABC transporter polypeptide is capable of being expressed as a fusion polypeptide with said gfp.

75. A gene construct comprising the isolated nucleic acid of claim 53 encoding a modified ABC transporter polypeptide wherein said nucleic acid is in operable connection with a promoter sequence to facilitate expression of said polypeptide in a cell.

76. The gene construct of claim 75 wherein the nucleic acid is in the same reading frame as nucleic acid encoding green fluorescent protein (gfp) such that the modified ABC transporter polypeptide is capable of being expressed as a fusion polypeptide with said gfp.

77. A gene construct comprising the isolated nucleic acid of claim 54 encoding a modified ABC transporter polypeptide wherein said nucleic acid is in operable connection with a promoter sequence to facilitate expression of said polypeptide in a cell.

78. The gene construct of claim 77 wherein the nucleic acid is in the same reading frame as nucleic acid encoding green fluorescent protein (gfp) such that the modified ABC transporter polypeptide is capable of being expressed as a fusion polypeptide with said gfp.

79. A gene construct comprising the isolated nucleic acid of claim 55 encoding a modified ABC transporter polypeptide wherein said nucleic acid is in operable connection with a promoter sequence to facilitate expression of said polypeptide in a cell.

80. The gene construct of claim 79 wherein the nucleic acid is in the same reading frame as nucleic acid encoding green fluorescent protein (gfp) such that the modified ABC transporter polypeptide is capable of being expressed as a fusion polypeptide with said gfp.

81. A gene construct comprising the isolated nucleic acid of claim 56 encoding a modified ABC transporter polypeptide wherein said nucleic acid is in operable connection with a promoter sequence to facilitate expression of said polypeptide in a cell.

82. The gene construct of claim 81 wherein the nucleic acid is in the same reading frame as nucleic acid encoding green fluorescent protein (gfp) such that the modified ABC transporter polypeptide is capable of being expressed as a fusion polypeptide with said gfp.

83. A method of enhancing the resistance of a cell to one or more chemical compounds comprising expressing a modified ABC transporter polypeptide in said cell for a time and under conditions sufficient for said cell to have modified growth and/or viability in the presence of said compound, wherein said modified ABC transporter comprises the amino acid sequence of the corresponding native ABC transporter wherein one or more amino acid residues of a C-terminal tripeptide T-K-F motif of said native ABC transporter polypeptide having a sequence set forth in any one of SEQ ID NOs: 52 to 58 is substituted or deleted.

84. The method of claim 83 wherein the native ABC transporter is selected from the group consisting of canalicular multispecific organic anion transporter (cMOAT), MDR3 and MRP4.

85. The method of claim 83 wherein said cell is a polarized cell and wherein said modified ABC transporter is localized predominantly in the basolateral membrane of said polarized cell.

86. The method of claim 85 wherein the polarized cell is an epithelial cell.

87. The method of claim 86 wherein the epithelial cell is selected from the group consisting of: a cultured MDCK cell, a cultured Caco-2 cell, a hepatocyte, an intestinal cell, and a hippocampal neuron.

88. The method of claim 83 wherein said cell is a non-polarized cell and wherein said modified ABC transporter is localized in the plasma membrane of said non-polarized cell.

89. The method of claim 88 wherein the non-polarized cell is selected from the group consisting of a fibroblast, a haemopoietic cell, a cultured L1210 cell and a cultured Jurkat cell.

90. The method of claim 83 wherein the chemical compound is a cytotoxic or cytostatic compound selected from the group consisting of:
(i) a chemotherapeutic agent that is capable of being transported from the cell by the modified ABC transporter;
(ii) an anti-bacterial compound that is capable of being transported from the cell by the modified ABC transporter; and
(iii) an anti-fungal compound that is capable of being transported from the cell by the modified ABC transporter.

91. The method of claim 83 wherein the efflux of the chemical compound from the cell is enhanced by the expressed modified ABC transporter thereby enhancing resistance of the cell to said chemical compound.

92. The method of claim 91 wherein the chemical compound is transported from the cell as a glutathione conjugate.

93. The method of claim 83 further comprising introducing nucleic acid into the cell encoding the modified ABC transporter.

94. A method of enhancing the resistance of a non-polarized cell to Busulfan comprising expressing a modified cMOAT polypeptide in said cell for a time and under conditions sufficient for said cell to have modified growth and/or viability in the presence of Busulfan, wherein said modified cMOAT polypeptide comprises the amino acid sequence of native cMOAT wherein one or more amino acid residues of the C-terminal tripeptide T-K-F motif of said native cMOAT having a sequence set forth in SEQ ID NO: 52 or SEQ ID NO: 53 or SEQ ID NO: 55 is substituted or deleted.

95. The method of claim 94 wherein the modified cMOAT polypeptide comprises the amino acid sequence of native cMOAT wherein one or more amino acid residues of the C-terminal tripeptide T-K-F motif of SEQ ID NO: 52 is substituted or deleted, said substitution or deletion selected from the group consisting of:
(i) substitution of threonine at the first position of the tripeptide T-K-F motif for alanine;
(ii) substitution of lysine at the second position of the tripeptide T-K-F motif for a different amino acid residue;
(iii) substitution of all three amino acid residues of the tripeptide T-K-F motif for different amino acid residues; and
(iv) deletion of the three amino acid residues of the tripeptide T-K-F motif.

96. The method of claim 95 wherein the different amino acid residue at (ii) is alanine or proline.

97. The method of claim 95 wherein each different amino acid residue at (iii) is alanine.

98. The method of claim 95 wherein the different amino acid residues at (iii) are alanine, proline and valine, respectively.

99. The method of claim 94 further comprising introducing nucleic acid into the non-polarized cell encoding the modified cMOAT polypeptide.

100. The method of claim 99 wherein the nucleic acid encoding the modified cMOAT polypeptide consists of the nucleotide sequence set forth in SEQ ID NO: 3 or a degenerate nucleotide sequence thereto.

101. A method of enhancing the resistance of a non-polarized cell to Busulfan comprising expressing a modified cMOAT polypeptide in said cell for a time and under conditions sufficient for said cell to have modified growth and/or viability in the presence of Busulfan, wherein said modified cMOAT polypeptide consists of the amino acid sequence set forth in SEQ ID NO: 4.

102. The method of claim 101 further comprising introducing nucleic acid into the non-polarized cell encoding the modified cMOAT polypeptide.

103. The method of claim 102 wherein the nucleic acid encoding the modified cMOAT polypeptide consists of the nucleotide sequence set forth in SEQ ID NO: 3 or a degenerate nucleotide sequence thereto.

104. A method of protecting a non-polarized cell of an organism or tissue comprising said non-polarized cell during the administration of a cytotoxic or cytostatic chemical compound to a subject, said method comprising:
(i) expressing the modified ABC transporter polypeptide of claim 1 in said non-polarized cell for a time and under conditions sufficient for said cell to efficiently transport said cytotoxic or cytostatic compound from said cell or otherwise acquire resistance to said compound; and
(ii) optionally administering an amount of an inhibitor of a native ABC transporter sufficient to ablate or inhibit the growth of a cell expressing said native ABC transporter, wherein said native ABC transporter is different to that from which said modified ABC transporter polypeptide is derived and is involved in the transport of said cytotoxic or cytostatic chemical compound and wherein said cell expressing said native ABC transporter is different to the non-polarized cell expressing the modified ABC transporter.

105. The method of claim 104 wherein the non-polarized cell expressing the modified ABC transporter is a cell of the haematopoietic system and wherein the cell expressing the native ABC transporter is an epithelial cell.

106. The method of claim 105 wherein the epithelial cell is selected from the group consisting of: hepatocyte, intestinal cell, and hippocampal neuron.

107. The method of claim 104 wherein the cell expressing the native ABC transporter is a polarized tumor cell or non-polarized tumor cell.

108. The method of claim 104 wherein the modified ABC transporter at (i) is a modified cMOAT polypeptide and wherein the native ABC transporter at (ii) is selected from the group consisting of native MRP1, native MRP3, native MRP4, native MRP5, native MRP6, native MDR3, and native P-gp.

109. The method of claim 108 the modified ABC transporter at (i) is a modified cMOAT polypeptide and wherein the native ABC transporter at (ii) is native MRP1 and wherein the cytostatic or cytotoxic compound is a substrate of MRP1 or MRP2.

110. The method of claim 108 wherein the modified ABC transporter at (i) is a modified cMOAT polypeptide and wherein the native ABC transporter at (ii) is native MRP3 and wherein the cytostatic or cytotoxic compound is a substrate of MRP2 or MRP3.

111. The method of claim 104 wherein the modified ABC transporter at (i) is a modified MDR3 polypeptide and wherein the native ABC transporter at (ii) is selected from the group consisting of native MRP1, native MRP2, native MRP3, native MRP4, native MRP5, native MRP6, and native P-gp.

112. The method of claim 111 wherein the native ABC transporter at (ii) is native MRP2 and wherein the inhibitor of native MRP2 is selected from the group consisting of: α-Naphthylisothiocyanate, Chlorpromazine, Cyclosporin, Estradiol-17β-glucuronide, Ethinylestradiol, Glycolithocholate-3α-O-sulfate, Lithocholate-3α-glucuronide, Manganese-bilirubin, Phalloidin, Taurocholate, and Taurolithocholate.

113. The method of claim 104 wherein the modified ABC transporter at (i) is a modified MRP4 polypeptide and wherein the native ABC transporter at (ii) is selected from the group consisting of native MRP1, native MRP2, native MRP3, native MRP5, native MRP6, native MDR3 and native P-gp.

114. The method of claim 113 wherein the native ABC transporter at (ii) is native MRP2 and wherein the inhibitor of native MRP2 is selected from the group consisting of: α-Naphthylisothiocyanate, Chlorpromazine, Cyclosporin, Estradiol-17β-glucuronide, Ethinylestradiol, Glycolithocholate-3α-O-sulfate, Lithocholate-3α-O-glucuronide, Manganese-bilirubin, Phalloidin, Taurocholate, and Taurolithocholate.

115. A method of enhancing the resistance of a polarized cell of an organism or tissue comprising said polarized cell during the administration of a cytotoxic or cytostatic chemical compound to a subject, said method comprising:
(i) expressing the modified ABC transporter polypeptide of claim 1 in said polarized cell for a time and under conditions sufficient for said cell to enhance transport said cytotoxic or cytostatic compound from said cell or otherwise enhance resistance to said compound; and
(ii) optionally, administering an amount of an inhibitor of a native ABC transporter sufficient to ablate or inhibit the growth of a cell expressing said native ABC transporter, wherein said native ABC transporter is different to that from which said modified ABC transporter polypeptide is derived and is involved in the transport of said cytotoxic or cytostatic chemical compound and wherein said cell expressing said native ABC transporter is different to the polarized cell expressing the modified ABC transporter.

116. The method of claim 115 wherein the polarized cell expressing the modified ABC transporter is an epithelial cell and wherein the cell expressing the native ABC transporter is a non-polarized cell.

117. The method of claim 116 wherein the epithelial cell is selected from the group consisting of: hepatocyte, intestinal cell, and hippocampal neuron.

118. The method of claim 116 wherein the cell expressing the native ABC transporter is a non-polarized cell of the haematopoletic system.

119. The method of claim 115 wherein the modified ABC transporter at (i) is a modified cMOAT polypeptide and wherein the native ABC transporter at (ii) is selected from the group consisting of native MRP1, native MRP3, native MRP4, native MRP5, native MRP6, native MDR3, and native P-gp.

120. The method of claim 115 wherein the modified ABC transporter at (i) is a modified cMOAT polypeptide and wherein the native ABC transporter at (ii) is native MRP1 and wherein the cytostatic or cytotoxic compound is a substrate of MRP1 or MRP2.

121. The method of claim 115 wherein the modified ABC transporter at (i) is a modified cMOAT polypeptide and wherein the native ABC transporter at (ii) is native MRP3 and wherein the cytostatic or cytotoxic compound is a substrate of MRP2 or MRP3.

122. The method of claim 115 wherein the modified ABC transporter at (i) is a modified MDR3 polypeptide and wherein the native ABC transporter at (ii) is selected from the group consisting of native MRP1, native MRP2, native MRP3, native MRP4, native MRP5, native MRP6, and native P-gp.

123. The method of claim 122 wherein the native ABC transporter at (ii) is native MRP2 and wherein the inhibitor of native MRP2 is selected from the group consisting of α-Naphthylisothiocyanate, Chlorpromazine, Cyclosporin, Estradiol-17β-glucuronide, Ethinylestradiol, Glycolithocholate-3α-O-sulfate, Lithocholate-3α-O-glucuronide, Manganese-bilirubin, Phalloidin, Taurocholate, and Taurolithocholate.

124. The method of claim 115 wherein the modified ABC transporter at (i) is a modified MRP4 polypeptide and wherein the native ABC transporter at (ii) is selected from the group consisting of native MRP1, native MRP2, native MRP3, native MRP5, native MRP6, native MDR3 and native P-gp.

125. The method of claim 124 wherein the native ABC transporter at (ii) is native MRP2 and wherein the inhibitor of native MRP2 is selected from the group consisting of: α-Naphthylisothiocyanate, Chlorpromazine, Cyclosporin, Estradiol-17β-glucuronide, Ethinylestradiol, Glycolithocholate-3α-O-sulfate, Lithocholate-3α-O-glucuronide, Manganese-bilirubin, Phalloidin, Taurocholate, and Taurolithocholate.

126. An isolated cell transformed with the gene construct of claim 67, wherein said cell expresses a modified ABC transporter polypeptide.

127. An isolated cell transformed with the gene construct of claim 68, wherein said cell expresses a modified ABC transporter polypeptide.

128. An isolated cell transformed with the gene construct of claim 69, wherein said cell expresses a modified ABC transporter polypeptide.

129. An isolated cell transformed with the gene construct of claim 70, wherein said cell expresses a modified ABC transporter polypeptide.

130. An isolated cell transformed with the gene construct of claim 71, wherein said cell expresses a modified ABC transporter polypeptide.

131. An isolated cell transformed with the gene construct of claim 72, wherein said cell expresses a modified ABC transporter polypeptide.

132. An isolated cell transformed with the gene construct of claim 73, wherein said cell expresses a modified ABC transporter polypeptide.

133. An isolated cell transformed with the gene construct of claim 74, wherein said cell expresses a modified ABC transporter polypeptide.

134. An isolated cell transformed with the gene construct of claim 75, wherein said cell expresses a modified ABC transporter polypeptide.

135. An isolated cell transformed with the gene construct of claim 76, wherein said cell expresses a modified ABC transporter polypeptide.

136. An isolated cell transformed with the gene construct of claim 77, wherein said cell expresses a modified ABC transporter polypeptide.

137. An isolated cell transformed with the gene construct of claim 78, wherein said cell expresses a modified ABC transporter polypeptide.

138. An isolated cell transformed with the gene construct of claim 79, wherein said cell expresses a modified ABC transporter polypeptide.

139. An isolated cell transformed with the gene construct of claim 80, wherein said cell expresses a modified ABC transporter polypeptide.

140. An isolated cell transformed with the gene construct of claim 81, wherein said cell expresses a modified ABC transporter polypeptide.

141. An isolated cell transformed with the gene construct of claim 82, wherein said cell expresses a modified ABC transporter polypeptide.

142. An isolated MDCK cell having a modified cMOAT polypeptide predominantly in the basolateral membrane, said modified cMOAT polypeptide having an amino acid sequence selected from the group consisting of (1) a sequence consisting of SEQ ID NO: 4;
(ii) a sequence comprising SEQ ID NO: 6;
(iii) a sequence comprising SEQ ID NO: 10; and
(iv) a sequence comprising SEQ ID NO: 16.

143. An isolated L1210 cell having a modified cMOAT polypeptide predominantly in the plasma membrane, said modified cMOAT polypeptide having an amino acid sequence selected from the group consisting of:
(i) a sequence consisting of SEQ ID NO: 4;
(ii) a sequence comprising SEQ ID NO: 6;
(iii) a sequence comprising SEQ ID NO: 10; and
(iv) a sequence comprising SEQ ID NO: 16.

144. The isolated L1210 cell of claim 143 wherein said cell has enhanced resistance to Busulfan compared to an L1210 cell not expressing said modified cMOAT polypeptide.

145. An isolated MDCK cell having a modified MDR3 polypeptide predominantly in the basolateral membrane, said modified MDR3 polypeptide having the amino acid sequence of SEQ ID NO: 49.

146. An isolated L1210 cell having a modified MDR3 polypeptide predominantly in the plasma membrane, said modified MDR3 polypeptide having the amino acid sequence of SEQ ID NO: 49.

147. An isolated MDCK cell having a modified MRP4 polypeptide predominantly In the basolateral membrane, said modified MRP4 polypeptide having the amino acid sequence of SEQ ID NO: 51.

148. An isolated L1210 cell having a modified MRP4 polypeptide predominantly in the plasma membrane, said modified MRP4 polypeptide having the amino acid sequence of SEQ ID NO: 51.

149. An isolated cell transformed with nucleic acid encoding a modified ABC transporter polypeptide, said modified ABC transporter polypeptide consisting of the amino acid sequence of a native ABC transporter polypeptide that normally accumulates in the apical (canalicular) membrane of a polarized cell wherein one or more amino acid residues of a C-terminal tripeptide T-K-F motif of said native ABC transporter polypeptide having a sequence set forth in any one of SEQ ID NOs: 52 to 58 is substituted or deleted.

150. The isolated cell of claim 148 wherein said cell is a polarized cell and wherein said modified ABC transporter accumulates predominantly in the basolateral membrane of said cell.

151. The isolated cell of claim 148, wherein said cell is a non-polarized cell and wherein said modified ABC transporter accumulates predominantly in the plasma membrane of said cell.

152. A process for identifying a substrate of a native ABC transporter polypeptide that normally accumulates in the apical (canalicular) membrane of a polarized cell, said process comprising:
(i) expressing the corresponding modified ABC transporter polypeptide In a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted; and
(iii) determining the efflux of the compound from the cell expressing the modified ABC transporter relative to a cell that does not express the native ABC transporter or the corresponding modified ABC transporter, wherein efflux from the cell expressing the modified ABC transporter indicates that the compound is a substrate for the corresponding native ABC transporter.

153. The process of claim 152 wherein efflux is determined by measuring the amount of a conjugate of the compound that is exported from the cell.

154. The process of claim 153 wherein the conjugate is a glutathione conjugate.

155. The process of claim 152 wherein the cell is a L1210 cell.

156. The process of claim 152 wherein the cell is an MDCK cell.

157. A process for identifying an antagonist of a native ABC transporter polypeptide that normally accumulates in the apical (canalicular) membrane of a polarized cell, said process comprising:
(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted; and;
(ii) incubating the cell in the presence of (a) a compound being tested for its ability to antagonize activity of the native ABC transporter polypeptide; and (b) a known substrate compound for said native ABC transporter polypeptide;
(iii) in a separate sample to (ii), incubating the cell in the presence of said substrate compound; and
(iv) comparing the efflux of the substrate compound at (ii) and (iii), wherein reduced efflux at (ii) compared to (iii) indicates that the compound being tested is an antagonist of said native ABC transporter polypeptide.

158. The process of claim 157 wherein the known substrate is selected from the group consisting of leukotriene C4 (LTC4); bilirubin; monoglucuronosyl bilirubin; bisglucuronosyl bilirubin; leukotriene D4 (LTD4); 1,3-chloro-2,4 dinitrobenzene; mono-chlorobimane (thiolyte); 7-chloro-4-nitrobenz-2-oxa-1,3-diazole; 17β-glucuronosyl estradiol; 3α-sulfatolithocholyl taurine; Fluo-3; glutathione disulphide; p-aminohippurate; digoxin; paclitaxel; verapamil; vinblastine; phosphatidylcholine; short chain phosphatidylcholine analogue; [α-³²P]8-azido-ATP; [α-³²P]ATP; [³H]verapamil; azidothymidine monophosphate; 9-(2-phosphonylmethoxyethyl)adenine (PMEA); 6-mercaptopurine; cAMP; cGMP; Sildenafil (Pfizer); Trequinsin (Sigma); and Zaprinast (Sigma).

159. The process of claim 158 wherein the substrate is selected from the group consisting of 1-chloro-2,4-dinitrobenzene; mono-chlorobimane (thiolyte); 7-chloro-4-nitrobenz-2-oxa-1,3-diazole;); 6-mercaptopurine; and paclitaxel.

160. The process of claim 157 wherein the cell is a L1210 cell.

161. The process of claim 157 wherein the cell is an MDCK cell.

162. A process for identifying an agonist of a native ABC transporter polypeptide that normally accumulates in the apical (canalicular) membrane of a polarized cell, said process comprising:
(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted; and;
(ii) incubating the cell in the presence of (a) a compound being tested for its ability to agonize activity of the native ABC transporter polypeptide;
and (b) a known substrate compound for said native ABC transporter polypeptide;
(iii) in a separate sample to (ii), incubating the cell in the presence of said substrate compound; and
(iv) comparing the efflux of the substrate compound at (ii) and (iii), wherein enhanced efflux at (ii) compared to (iii) indicates that the compound being tested is an agonist of said native ABC transporter polypeptide.

163. The process of claim 162 wherein the known substrate is selected from the group consisting of: leukotriene C4 (LTC4); bilirubin; monoglucuronosyl bilirubin; bisglucuronosyl bilirubin; leukotriene D4 (LTD4); 1,3-chloro-2,4-dinitrobenzene; mono-chlorobimane (thiolyte); 7-chloro-4-nitrobenz-2-oxa-1,3-diazole; 17β-glucuronosyl estradiol; 3α-sulfatolithocholyl taurine; Fluo-3; glutathione disulphide; p-aminohippurate; digoxin; paclitaxel; verapamil; vinblastine; phosphatidylcholine; short chain phosphatidylcholine analogue; [α-³²P]8-azido-ATP; [α-³²P]ATP; [³H]verapamil; azidothymidine monophosphate; 9-(2-phosphonylmethoxyethyl)adenine (PMEA); 6-mercaptopurine; cAMP; cGMP; Sildenafil (Pfizer); Trequinsin (Sigma); and Zaprinast (Sigma).

164. The process of claim 163 wherein the substrate is selected from the group consisting of 1-chloro-2,4-dinitrobenzene; mono-chlorobimane (thiolyte); 7-chloro-4-nitrobenz-2-oxa-1,3-diazole;); 6-mercaptopurine; and paclitaxel.

165. The process of claim 162 wherein the cell is a L1210 cell.

166. The process of claim 162 wherein the cell is an MDCK cell.

167. A process for identifying a modulator of a native ABC transporter polypeptide that normally accumulates in the apical (canalicular) membrane of a polarized cell, said process comprising:
(i) expressing the corresponding modified ABC transporter polypeptide in a cell, wherein said modified ABC transporter polypeptide consists of the amino acid sequence of said native ABC transporter polypeptide wherein one or more amino acid residues of a C-terminal T-K-F motif of said native ABC transporter polypeptide having a sequence set forth in any one of SEQ ID NOs: 52 to 58 is substituted or deleted; and;
(ii) incubating the cell in the presence of 1-chloro-2,4-dinitrobenzene;
(iii) incubating the cell at (ii) in the presence of (a) the compound being tested for its ability to modulate activity of the native ABC transporter polypeptide; and
(iv) comparing the efflux of 2,4-dinitro-phenylglutathione (DNP-GS) at (ii) and (iii), wherein enhanced efflux at (ii) compared to (iii) indicates that the compound being tested is an agonist of said native ABC transporter polypeptide and wherein reduced efflux at (ii) compared to (iii) indicates that the compound being tested is an antagonist of said native ABC transporter polypeptide.

168. The process of claim 167 wherein the cell is a L1210 cell.

169. The process of claim 167 wherein the cell is an MDCK cell.

170. A process for identifying a modulator of a native canalicular multispecific organic anion transporter (cMOAT) polypeptide, said process comprising:
(i) expressing a modified cMOAT polypeptide in an L1210 cell, wherein said modified cMOAT polypeptide comprises the amino acid sequence of native cMOAT wherein one or more amino acid residues of the C terminal tripeptide T-K-F motif of said native cMOAT having a sequence set forth in SEQ ID NO: 52 or SEQ ID NO: 53 or SEQ ID NO: 55 is substituted or deleted;
(ii) incubating the cell in the presence of 1-chloro-2,4-dinitrobenzene;
(iii) incubating the cell at (ii) in the presence of (a) the compound being tested for its ability to modulate activity of the native cMOAT polypeptide; and
(iv) comparing the efflux of 2,4-dinitro-phenylglutathione (DNP-GS) at (ii) and (iii), wherein enhanced efflux at (ii) compared to (iii) indicates that the compound being tested is an agonist of said native cMOAT polypeptide and wherein reduced efflux at (ii) compared to (iii) indicates that the compound being tested is an antagonist of said native cMOAT polypeptide.

171. The process of claim 170 wherein said modified cMOAT polypeptide has an amino acid sequence selected from the group consisting of:
(i) a sequence consisting of SEQ ID NO: 4;
(ii) a sequence comprising SEQ ID NO: 6;
(iii) a sequence comprising SEQ ID NO: 10; and
(iv) a sequence comprising SEQ ID NO: 16.

172. A process for identifying a modulator of a native MDR3 polypeptide, said process comprising:
(i) expressing a modified MDR3 polypeptide in an L1210 cell, wherein said modified MDR3 polypeptide consists of the amino acid sequence of SEQ ID NO: 49;
(ii) incubating the cell in the presence of [³H]paclitaxel;
(iii) incubating the cell at (ii) in the presence of (a) the compound being tested for its ability to modulate activity of the native MDR3 polypeptide; and
(iv) comparing the efflux of [³H]paclitaxel at (ii) and (iii), wherein enhanced efflux at (ii) compared to (iii) indicates that the compound being tested is an agonist of said native MDR3 polypeptide and wherein reduced efflux at (ii) compared to (iii) indicates that the compound being tested is an antagonist of said native MDR3 polypeptide.

173. A process for identifying a modulator of a native MRP4 polypeptide, said process comprising:
(I) expressing a modified MRP4 polypeptide in an L1210 cell, wherein said modified MRP4 polypeptide consists of the amino acid sequence of SEQ ID NO: 51;
(ii) incubating the cell in the presence of 6-mercaptopurine;
(iii) incubating the cell at (ii) in the presence of (a) the compound being tested for its ability to modulate activity of the native MRP4 polypeptide; and
(iv) comparing the efflux of 6-thio-IMP at (ii) and (iii), wherein enhanced efflux at (ii) compared to (iii) indicates that the compound being tested is an agonist of said native MRP4 polypeptide and wherein reduced efflux at (ii) compared to (iii) indicates that the compound being tested is an antagonist of said native MRP4 polypeptide.


SEQUENCE LISTING (62 sequences)

SEQ ID NO: 1 - DNA, 4635 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcacaaaattc

SEQ ID NO: 2 - protein, 1545 aa, Homo sapiens [sequence omitted]

SEQ ID NO: 3 - DNA, 4638 bp, Homo sapiens
  CDS (1)..(4626)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagctagcagaaggcc

SEQ ID NO: 4 - protein, 1542 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNS

SEQ ID NO: 5 - DNA, 4638 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcgcaccggtcgcc

SEQ ID NO: 6 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNSAPV

SEQ ID NO: 7 - DNA, 4637 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacgccacaaaattcgc

SEQ ID NO: 8 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNATKF

SEQ ID NO: 9 - DNA, 4640 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcgcaaaattcgcacc

SEQ ID NO: 10 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNSAKF

SEQ ID NO: 11 - DNA, 4641 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcacagcattcgcaccg

SEQ ID NO: 12 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNSTAF

SEQ ID NO: 13 - DNA, 4645 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcacaaaagccgcaccggtcg

SEQ ID NO: 14 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNSTKA

SEQ ID NO: 15 - DNA, 4645 bp, Homo sapiens
  CDS (1)..(4635)
atgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcgcagcagccgcaccggtcg

SEQ ID NO: 16 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEERIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIEYGSPEELLQIPGPFYFMAKEAGIENVNSAAA

SEQ ID NO: 17 - protein, 42 aa, Homo sapiens
VMVLDNGKIIEYGSPEELLQIPGPFYFMAKEAGIENVNSTKF

SEQ ID NO: 18 - protein, 42 aa, R. rattus
IMVLDNGKIVEYGSPEELLSNRGSFYLMAKEAGIENVNHTEL

SEQ ID NO: 19 - protein, 43 aa, Oryctolagus
IMVLDNGNIVEYGSPEELLESAGPFSLMAKESGIENVNNTAFW

SEQ ID NO: 20 - protein, 25 aa, Homo sapiens
VVNGRVKHGTHAKGYSMVSVAGTKR

SEQ ID NO: 21 - protein, 36 aa, R. rattus
IVVIQNGQVKEHGTHQQLLAQKGIYFSMVQAGAKRS

SEQ ID NO: 22 - protein, 38 aa, Homo sapiens
IVVFQNGRVKEHGTHQQLLAQKGIYFSMVSVQAGTQNL

SEQ ID NO: 23 - protein, 34 aa, Homo sapiens
IVLDKGEIQEYGAPSDLLQQRGLFYSMAKDAGLV

SEQ ID NO: 24 - DNA, 31 bp, Artificial Sequence
  Description of Artificial Sequence oligonucleotide primer for amplifying human cMOAT
agcgctagcgatgctggagaagttctgcaac

SEQ ID NO: 25 - DNA, 36 bp, Artificial Sequence
  Description of Artificial Sequence oligonucleotide primer for amplifying human cMOAT
tacggtaccggtgcgaattttgtgctgttcacattc

SEQ ID NO: 26 - DNA, 25 bp, Artificial Sequence
  Description of Artificial Sequence oligonucleotide primer for amplifying delta cMOAT (no gfp fusion)
gaatgtgaacagcacaaaattcgcc

SEQ ID NO: 27 - DNA, 25 bp, Artificial Sequence
  Description of Artificial Sequence oligonucleotide primer for amplifying T1543A K1544P F1545V
gaatgtgaacagcgcaccggtcgcc

SEQ ID NO: 28 - DNA, 24 bp, Artificial Sequence
  Description of Artificial Sequence oligonucleotide primer for amplifying S1542A
gaatgtgaacgccacaaaattcgc

SEQ ID NO: 29 - DNA, 24 bp, Artificial Sequence
  Description of Artificial Sequence oligonucleotide primer for amplifying T1543A
tgtgaacagcgcaaaattcgcacc

SEQ ID NO: 30 - DNA, 24 bp, Artificial Sequence
  Description of Artificial Sequence oligonucleotide primer for amplifying K1544A
gtgaacagcacagcattcgcaccg

SEQ ID NO: 31 - DNA, 23 bp, Artificial Sequence
  Description of Artificial Sequence oligonucleotide primer for amplifying F1545A
cagcacaaaagccgcaccggtcg

SEQ ID NO: 32 - DNA, 30 bp, Artificial Sequence
  Description of Artificial Sequence oligonucleotide primer for amplifying T1543A K1544A F1545A
atgtgaacagcgcagcagccgcaccggtcg

SEQ ID NO: 33 - DNA, 25 bp, Artificial Sequence
  Description of Artificial Sequence oligonucleotide primer for amplifying delta cMOAT (for gfp fusion)
gaatgtgaacagctagcagaaggcc

SEQ ID NO: 34 - DNA, 28 bp, Artificial Sequence
  Description of Artificial Sequence oligonucleotide primer for amplifying human MRP1
gcggccgcggatggcgctccggggcttc

SEQ ID NO: 35 - DNA, 34 bp, Artificial Sequence
  Description of Artificial Sequence oligonucleotide primer for amplifying human MRP1
tacggtaccggtgccaccaagccggcgtctttgg

SEQ ID NO: 36 - DNA, 25 bp, Artificial Sequence
  Description of Artificial Sequence oligonucleotide primer for amplifying non-tagged delta cMOAT
ggccttctgctagctgttcacattc

SEQ ID NO: 37 - protein, 7 aa, Homo sapiens
NVNSTKF

SEQ ID NO: 38 - protein, 7 aa, Artificial Sequence
  Description of Artificial Sequence cMOAT T1543A K1544P F1545V mutant C-terminus
NVNSAPV

SEQ ID NO: 39 - protein, 7 aa, Artificial Sequence
  Description of Artificial Sequence cMOAT S1542A mutant C-terminus
NVNATKF

SEQ ID NO: 40 - protein, 6 aa, Artificial Sequence
  Description of Artificial Sequence cMOAT T1543A mutant C-terminus
VNSAKF

SEQ ID NO: 41 - protein, 6 aa, Artificial Sequence
  Description of Artificial Sequence cMOAT K1544A mutant C-terminus
VNSTAF

SEQ ID NO: 42 - protein, 4 aa, Artificial Sequence
  Description of Artificial Sequence cMOAT F1545A mutant C-terminus
STKA

SEQ ID NO: 43 - protein, 6 aa, Artificial Sequence
  Description of Artificial Sequence cMOAT T1543A K1544A F1545A mutant C-terminus
VNSAAA

SEQ ID NO: 44 - protein, 4 aa, Artificial Sequence
  Description of Artificial Sequence delta cMOAT C-terminus
NVNS

SEQ ID NO: 45 - protein, 40 aa, Artificial Sequence
  Description of Artificial Sequence HisP C-terminus
VIFLHQGKIEEEGDPEQVFGNPQSPRLQQFLKGSLKKLEH

SEQ ID NO: 46 - protein, 42 aa, Mus musculus
VMVLDSGKIVEYGSPEELLSNMGPFYLMAKEAGIESVNHTEL

SEQ ID NO: 47 - protein, 82 aa, Homo sapiens
IMVLDSGRLKEYDEPYVLLQNKESLFYKMVQQLGKAEAAALTETAKQVYFKRNYPHIGHTDHMVTNTSNGQPSTLTIFETAL

SEQ ID NO: 48 - DNA, 3825 bp, Homo sapiens
  CDS (1)..(3825)
atggatcttgaggcggcaaagaacggaacagcctggcgccccacgagcgcggagggcgactttgaactgggcatcagcagcaaacaaaaaaggaaaaaaacgaagacagtgaaaatgattggagtattaacattgtttcgatactccgattggcaggataaattgtttatgtcgctgggtaccatcatggccatagctcacggatcaggtctccccctcatgatgatagtatttggagagatgactgacaaatttgttgatactgcaggaaacttctcctttccagtgaacttttccttgtcgctgctaaatccaggcaaaattctggaagaagaaatgactagatatgcatattactactcaggattgggtgctggagttcttgttgctgcctatatacaagtttcattttggactttggcagctggtcgacagatcaggaaaattaggcagaagttttttcatgctattctacgacaggaaataggatggtttgacatcaacgacaccactgaactcaatacgcggctaacagatgacatctccaaaatcagtgaaggaattggtgacaaggttggaatgttctttcaagcagtagccacgttttttgcaggattcatagtgggattcatcagaggatggaagctcacccttgtgataatggccatcagccctattctaggactctctgcagccgtttgggcaaagatactctcggcatttagtgacaaagaactagctgcttatgcaaaagcaggcgccgtggcagaagaggctctgggggccatcaggactgtgatagctttcgggggccagaacaaagagctggaaaggtatcagaaacatttagaaaatgccaaagagattggaattaaaaaagctatttcagcaaacatttccatgggtattgccttcctgttaatatatgcatcatatgcactggccttctggtatggatccactctagtcatatcaaaagaatatactattggaaatgcaatgacagtttttttttcaatcctaattggagctttcagtgttggccaggctgccccatgtattgatgcttttgccaatgcaagaggagcagcatatgtgatctttgatattattgataataatcctaaaattgacagtttttcagagagaggacacaaaccagacagcatcaaagggaatttggagttcaatgatgttcacttttcttacccttctcgagctaacgtcaagatcttgaagggcctcaacctgaaggtgcagagtgggcagacggtggccctggttggaagtagtggctgtgggaagagcacaacggtccagctgatacagaggctctatgaccctgatgagggcacaattaacattgatgggcaggatattaggaactttaatgtaaactatctgagggaaatcattggtgtggtgagtcaggagccggtgctgttttccaccacaattgctgaaaatatttgttatggccgtggaaatgtaaccatggatgagataaagaaagctgtcaaagaggccaacgcctatgagtttatcatgaaattaccacagaaatttgacaccctggttggagagagaggggcccagctgagtggtgggcagaagcagaggatcgccattgcacgtgccctggttcgcaaccccaagatccttctgctggatgaggccacgtcagcattggacacagaaagtgaagctgaggtacaggcagctctggataaggccagagaaggccggaccaccattgtgatagcacaccgactgtctacggtccgaaatgcagatgtcatcgctgggtttgaggatggagtaattgtggagcaaggaagccacagcgaactgatgaagaaggaaggggtgtacttcaaacttgtcaacatgcagacatcaggaagccagatccagtcagaagaatttgaactaaatgatgaaaaggctgccactagaatggccccaaatggctggaaatctcgcctatttaggcattctactcagaaaaaccttaaaaattcacaaatgtgtcagaagagccttgatgtggaaaccgatggacttgaagcaaatgtgccaccagtgtcctttctgaaggtcctgaaactgaataaaacagaatggccctactttgtcgtgggaacagtatgtgccattgccaatggggggcttcagccggcattttcagtcatattctcagagatcatagcgatttttggaccaggcgatgatgcagtgaagcagcagaagtgcaacatattctctttgattttcttatttctgggaattatttctttttttactttcttccttcagggtttcacgtttgggaaagctggcgagatcctcaccagaagactgcggtcaatggcttttaaagcaatgctaagacaggacatgagctggtttgatgaccataaaaacagtactggtgcactttctacaagacttgccacagatgctgcccaagtccaaggagccacaggaaccaggttggctttaattgcacagaatatagctaaccttggaactggtattatcatatcatttatctacggttggcagttaaccctattgctattagcagttgttccaattattgctgtgtcaggaattgttgaaatgaaattgttggctggaaatgccaaaagagataaaaaagaactggaagctgctggaaagattgcaacagaggcaatagaaaatattaggacagttgtgtctttgacccaggaaagaaaatttgaatcaatgtatgttgaaaaattgtatggaccttacaggaattctgtgcagaaggcacacatctatggaattacttttagtatctcacaagcatttatgtatttttcctatgccggttgttttcgatttggtgcatatctcattgtgaatggacatatgcgcttcagagatgttattctggtgttttctgcaattgtatttggtgcagtggctctaggacatgccagttcatttgctccagactatgctaaagctaagctgtctgcagcccacttattcatgctgtttgaaagacaacctctgattgacagctacagtgaagaggggctgaagcctgataaatttgaaggaaatataacatttaatgaagtcgtgttcaactatcccacccgagcaaacgtgccagtgcttcaggggctgagcctggaggtgaagaaaggccagacactagccctggtgggcagcagtggctgtgggaagagcacggtggtccagctcctggagcggttctacgaccccttggcggggacagtgcttctcgatggtcaagaagcaaagaaactcaatgtccagtggctcagagctcaactcggaatcgtgtctcaggagcctatcctatttgactgcagcattgccgagaatattgcctatggagacaacagccgggttgtatcacaggatgaaattgtgagtgcagccaaagctgccaacatacatcctttcatcgagacgttaccccacaaatatgaaacaagagtgggagataaggggactcagctctcaggaggtcaaaaacagaggattgctattgcccgagccctcatcagacaacctcaaatcctcctgttggatgaagctacatcagctctggatactgaaagtgaaaaggttgtccaagaagccctggacaaagccagagaaggccgcacctgcattgtgattgctcaccgcctgtccaccatccagaatgcagacttaatagtggtgtttcagaatgggagagtcaaggagcatggcacgcatcagcagctgctggcacagaaaggcatctatttttcaatggtcagtgtccaggctggg

SEQ ID NO: 49 - protein, 1275 aa, Homo sapiens
MDLEAAKNGTAWRPTSAEGDFELGISSKQKRKKTKTVKMIGVLTLFRYSDWQDKLFMSLGTIMAIAHGSGLPLMMIVFGEMTDKFVDTAGNFSFPVNFSLSLLNPGKILEEEMTRYAYYYSGLGAGVLVAAYIQVSFWTLAAGRQIRKIRQKFFHAILRQEIGWFDINDTTELNTRLTDDISKISEGIGDKVGMFFQAVATFFAGFIVGFIRGWKLTLVIMAISPILGLSAAVWAKILSAFSDKELAAYAKAGAVAEEALGAIRTVIAFGGQNKELERYQKHLENAKEIGIKKAISANISMGIAFLLIYASYALAFWYGSTLVISKEYTIGNAMTVFFSILIGAFSVGQAAPCIDAFANARGAAYVIFDIIDNNPKIDSFSERGHKPDSIKGNLEFNDVHFSYPSRANVKILKGLNLKVQSGQTVALVGSSGCGKSTTVQLIQRLYDPDEGTINIDGQDIRNFNVNYLREIIGVVSQEPVLFSTTIAENICYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTVRNADVIAGFEDGVIVEQGSHSELMKKEGVYFKLVNMQTSGSQIQSEEFELNDEKAATRMAPNGWKSRLFRHSTQKNLKNSQMCQKSLDVETDGLEANVPPVSFLKVLKLNKTEWPYFVVGTVCAIANGGLQPAFSVIFSEIIAIFGPGDDAVKQQKCNIFSLIFLFLGIISFFTFFLQGFTFGKAGEILTRRLRSMAFKAMLRQDMSWFDDHKNSTGALSTRLATDAAQVQGATGTRLALIAQNIANLGTGIIISFIYGWQLTLLLLAVVPIIAVSGIVEMKLLAGNAKRDKKELEAAGKIATEAIENIRTVVSLTQERKFESMYVEKLYGPYRNSVQKAHIYGITFSISQAFMYFSYAGCFRFGAYLIVNGHMRFRDVILVFSAIVFGAVALGHASSFAPDYAKAKLSAAHLFMLFERQPLIDSYSEEGLKPDKFEGNITFNEVVFNYPTRANVPVLQGLSLEVKKGQTLALVGSSGCGKSTVVQLLERFYDPLAGTVLLDGQEAKKLNVQWLRAQLGIVSQEPILFDCSIAENIAYGDNSRVVSQDEIVSAAKAANIHPFIETLPHKYETRVGDKGTQLSGGQKQRIAIARALIRQPQILLLDEATSALDTESEKVVQEALDKAREGRTCIVIAHRLSTIQNADLIVVFQNGRVKEHGTHQQLLAQKGIYFSMVSVQAG

SEQ ID NO: 50 - DNA, 3966 bp, Homo sapiens
  CDS (1)..(3966)
atgctgcccgtgtaccaggaggtgaagcccaacccgctgcaggacgcgaacctctgctcacgcgtgttcttctggtggctcaatcccttgtttaaaattggccataaacggagattagaggaagatgatatgtattcagtgctgccagaagaccgctcacagcaccttggagaggagttgcaagggttctgggataaagaagttttaagagctgagaatgacgcacagaagccttctttaacaagagcaatcataaagtgttactggaaatcttatttagttttgggaatttttacgttaattgaggaaagtgccaaagtaatccagcccatatttttgggaaaaattattaattattttgaaaattatgatcccatggattctgtggctttgaacacagcgtacgcctatgccacggtgctgactttttgcacgctcattttggctatactgcatcacttatatttttatcacgttcagtgtgctgggatgaggttacgagtagccatgtgccatatgatttatcggaaggcacttcgtcttagtaacatggccatggggaagacaaccacaggccagatagtcaatctgctgtccaatgatgtgaacaagtttgatcaggtgacagtgttcttacacttcctgtgggcaggaccactgcaggcgattgcagtgactgccctactctggatggagataggaatatcgtgccttgctgggatggcagttctaatcattctcctgcccttgcaaagctgttttgggaagttgttctcatcactgaggagtaaaactgcaactttcacggatgccaggatcaggaccatgaatgaagttataactggtataaggataataaaaatgtacgcctgggaaaagtcattttcaaatcttattaccaatttgagaaagaaggagatttccaagattctgagaagttcctgcctcagagggatgaatttggcttcatttttcagtgcaagcaaaatcatcgtgtttgtgaccttcaccacctacgtgctcctcggcagtgtgatcacagccagccgcgtgttcgtggcagtgacgctgtatggggctgtgcggctgacggttaccctcttcttcccctcagccattgagagggtgtcagaggcaatcgtcagcatccgaagaatccagacctttttgctacttgatgagatatcacagcgcaaccgtcagctgccgtcagatggtaaaaagatggtgcatgtgcaggattttactgctttttgggataaggcatcagagaccccaactctacaaggcctttcctttactgtcagacctggcgaattgttagctgtggtcggccccgtgggagcagggaagtcatcactgttaagtgccgtgctcggggaattggccccaagtcacgggctggtcagcgtgcatggaagaattgcctatgtgtctcagcagccctgggtgttctcgggaactctgaggagtaatattttatttgggaagaaatacgaaaaggaacgatatgaaaaagtcataaaggcttgtgctctgaaaaaggatttacagctgttggaggatggtgatctgactgtgataggagatcggggaaccacgctgagtggagggcagaaagcacgggtaaaccttgcaagagcagtgtatcaagatgctgacatctatctcctggacgatcctctcagtgcagtagatgcggaagttagcagacacttgttcgaactgtgtatttgtcaaattttgcatgagaagatcacaattttagtgactcatcagttgcagtacctcaaagctgcaagtcagattctgatattgaaagatggtaaaatggtgcagaaggggacttacactgagttcctaaaatctggtatagattttggctcccttttaaagaaggataatgaggaaagtgaacaacctccagttccaggaactcccacactaaggaatcgtaccttctcagagtcttcggtttggtctcaacaatcttctagaccctccttgaaagatggtgctctggagagccaagatacagagaatgtcccagttacactatcagaggagaaccgttctgaaggaaaagttggttttcaggcctataagaattacttcagagctggtgctcactggattgtcttcattttccttattctcctaaacactgcagctcaggttgcctatgtgcttcaagattggtggctttcatactgggcaaacaaacaaagtatgctaaatgtcactgtaaatggaggaggaaatgtaaccgagaagctagatcttaactggtacttaggaatttattcaggtttaactgtagctaccgttctttttggcatagcaagatctctattggtattctacgtccttgttaactcttcacaaactttgcacaacaaaatgtttgagtcaattctgaaagctccggtattattctttgatagaaatccaataggaagaattttaaatcgtttctccaaagacattggacacttggatgatttgctgccgctgacgtttttagatttcatccagacattgctacaagtggttggtgtggtctctgtggctgtggccgtgattccttggatcgcaatacccttggttccccttggaatcattttcatttttcttcggcgatattttttggaaacgtcaagagatgtgaagcgcctggaatctacaactcggagtccagtgttttcccacttatcatcttctctccaggggctctggaccatccgggcatacaaagcagaagagaggtgtcaggaactgtttgatgcacaccaggatttacattcagaggcttggttcttgtttttgacaacgtcccgctggtttgccgtccgtctggatgccatctgtgccatgtttgtcatcatcgttgcctttgggtccctgattctggcaaaaactctggatgccgggcaggttggtttggcactgtcctatgccctcacgctcatggggatgtttcagtggtgtgttcgacaaagtgctgaagttgagaatatgatgatctcagtagaaagggtcattgaatacacagaccttgaaaaagaagcaccttgggaatatcagaaacgcccaccaccagcctggccccatgaaggagtgataatctttgacaatgtgaacttcatgtacagtccaggtgggcctctggtactgaagcatctgacagcactcattaaatcacaagaaaaggttggcattgtgggaagaaccggagctggaaaaagttccctcatctcagccctttttagattgtcagaacccgaaggtaaaatttggattgataagatcttgacaactgaaattggacttcacgatttaaggaagaagatgtcaatcatacctcaggaacctgttttgttcactggaacaatgaggaaaaacctggatccctttaatgagcacacggatgaggaactgtggaatgccttacaagaggtacaacttaaagaaaccattgaagatcttcctggtaaaatggatactgaattagcagaatcaggatccaattttagtgttggacaaagacaactggtgtgccttgccagggcaattctcaggaaaaatcagatattgattattgatgaagcgacggcaaatgtggatccaagaactgatgagttaatacaaaaaaaaatccgggagaaatttgcccactgcaccgtgctaaccattgcacacagattgaacaccattattgacagcgacaagataatggttttagattcaggaagactgaaagaatatgatgagccgtatgttttgctgcaaaataaagagagcctattttacaagatggtgcaacaactgggcaaggcagaagccgctgccctcactgaaacagcaaaacaggtatacttcaaaagaaattatccacatattggtcacactgaccacatggttacaaacacttccaatggacagccctcgaccttaactattttcgag

SEQ ID NO: 51 - protein, 1322 aa, Homo sapiens
MLPVYQEVKPNPLQDANLCSRVFFWWLNPLFKIGHKRRLEEDDMYSVLPEDRSQHLGEELQGFWDKEVLRAENDAQKPSLTRAIIKCYWKSYLVLGIFTLIEESAKVIQPIFLGKIINYFENYDPMDSVALNTAYAYATVLTFCTLILAILHHLYFYHVQCAGMRLRVAMCHMIYRKALRLSNMAMGKTTTGQIVNLLSNDVNKFDQVTVFLHFLWAGPLQAIAVTALLWMEIGISCLAGMAVLIILLPLQSCFGKLFSSLRSKTATFTDARIRTMNEVITGIRIIKMYAWEKSFSNLITNLRKKEISKILRSSCLRGMNLASFFSASKIIVFVTFTTYVLLGSVITASRVFVAVTLYGAVRLTVTLFFPSAIERVSEAIVSIRRIQTFLLLDEISQRNRQLPSDGKKMVHVQDFTAFWDKASETPTLQGLSFTVRPGELLAVVGPVGAGKSSLLSAVLGELAPSHGLVSVHGRIAYVSQQPWVFSGTLRSNILFGKKYEKERYEKVIKACALKKDLQLLEDGDLTVIGDRGTTLSGGQKARVNLARAVYQDADIYLLDDPLSAVDAEVSRHLFELCICQILHEKITILVTHQLQYLKAASQILILKDGKMVQKGTYTEFLKSGIDFGSLLKKDNEESEQPPVPGTPTLRNRTFSESSVWSQQSSRPSLKDGALESQDTENVPVTLSEENRSEGKVGFQAYKNYFRAGAHWIVFIFLILLNTAAQVAYVLQDWWLSYWANKQSMLNVTVNGGGNVTEKLDLNWYLGIYSGLTVATVLFGIARSLLVFYVLVNSSQTLHNKMFESILKAPVLFFDRNPIGRILNRFSKDIGHLDDLLPLTFLDFIQTLLQVVGVVSVAVAVIPWIAIPLVPLGIIFIFLRRYFLETSRDVKRLESTTRSPVFSHLSSSLQGLWTIRAYKAEERCQELFDAHQDLHSEAWFLFLTTSRWFAVRLDAICAMFVIIVAFGSLILAKTLDAGQVGLALSYALTLMGMFQWCVRQSAEVENMMISVERVIEYTDLEKEAPWEYQKRPPPAWPHEGVIIFDNVNFMYSPGGPLVLKHLTALIKSQEKVGIVGRTGAGKSSLISALFRLSEPEGKIWIDKILTTEIGLHDLRKKMSIIPQEPVLFTGTMRKNLDPFNEHTDEELWNALQEVQLKETIEDLPGKMDTELAESGSNFSVGQRQLVCLARAILRKNQILIIDEATANVDPRTDELIQKKIREKFAHCTVLTIAHRLNTIIDSDKIMVLDSGRLKEYDEPYVLLQNKESLFYKMVQQLGKAEAAALTETAKQVYFKRNYPHIGHTDHMVTNTSNGQPSTLTIFE

SEQ ID NO: 52 - protein, 3 aa, Artificial Sequence
  Description of Artificial Sequence T-K-F peptide motif variant
TKF

SEQ ID NO: 53 - protein, 3 aa, Artificial Sequence
  Description of Artificial Sequence T-K-F peptide motif variant
TAF

SEQ ID NO: 54 - protein, 3 aa, Artificial Sequence
  Description of Artificial Sequence T-K-F peptide motif variant
TAL

SEQ ID NO: 55 - protein, 3 aa, Artificial Sequence
  Description of Artificial Sequence T-K-F peptide motif variant
TEL

SEQ ID NO: 56 - protein, 3 aa, Artificial Sequence
  Description of Artificial Sequence T-K-F peptide motif variant
TKR

SEQ ID NO: 57 - protein, 3 aa, Artificial Sequence
  Description of Artificial Sequence T-K-F peptide motif variant
TQN

SEQ ID NO: 58 - protein, 3 aa, Artificial Sequence
  Description of Artificial Sequence T-K-F peptide motif variant
AKR

SEQ ID NO: 59 - DNA, 31 bp, Artificial Sequence
  Description of Artificial Sequence oligonucleotide primer for amplifying modified human MDR3
agcgctagcgatggatcttgaggcggcaaag

SEQ ID NO: 60 - DNA, 27 bp, Artificial Sequence
  Description of Artificial Sequence oligonucleotide primer for amplifying modified human MDR3
tacggtaccggtgccccagcctggaca

SEQ ID NO: 61 - DNA, 31 bp, Artificial Sequence
  Description of Artificial Sequence oligonucleotide primer for amplifying modified human MRP4
agcgctagcgatgctgcccgtgtaccaggag

SEQ ID NO: 62 - DNA, 27 bp, Artificial Sequence
  Description of Artificial Sequence oligonucleotide primer for amplifying modified human MRP4
tacggtaccggtgcctcgaaaatagtt